(19) United States
(12) Patent Application Publication
(10) Pub. No.: US 2009/0264845 A1
(43) Pub. Date: Oct. 22, 2009

(54) Absorbent composite and method for producing same, asorbent article and nozzle

(75) Inventors: Shunichi Himori, Mie (JP); Kiichi Itoh, Mie (JP); Yasunari Sugyo, Mie (JP); Taisuke Ishii, Mie (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Mitsubishi Chemical, Tokyo (JP)

(21) Appl. No.: 11/658,785

(22) PCT Filed: Aug. 1, 2005
(86) PCT No.: PCT/JP2005/014056

§ 371 Date: Jun. 16, 2007

(30) Foreign Application Priority Data
Jul. 30, 2004 (JP) 2004 223961
Aug. 27, 2004 (JP) 2004248686
Dec. 17, 2004 (JP) 2004 366722
Dec. 20, 2004 (JP) 2004 367874

(51) Int. Cl. A61F 13/15; B32B 9/04; B32B 5/16; B32B 3/26; C23C 16/44; B32B 37/10; B05D 1/00; B05B 7/10

(52) U.S. Cl. 604/367; 428/411.1; 428/327; 428/339; 428/304.4; 427/255.6; 156/60; 427/206; 604/378; 239/399; 239/404

(57) ABSTRACT

An absorbent composite comprising a bound particulate absorbent polymer in which two or more nearly spherical absorbent polymer particles are bound to each other and a web-like absorbent polymer, wherein the bound particulate absorbent polymer and the web-like absorbent polymer are bound to a substrate. The composite has a large absorption and a high absorbing speed in which the absorbent polymer is kept uniformly dispersed on the support throughout before and after absorption.

### TECHNICAL FIELD

The present invention relates to an absorbent composite and a method for producing it. The absorbent composite of the invention is suitable for sanitary materials such as paper diapers and sanitary protections, industrial materials necessary for absorbing and holding wastewater, and agricultural materials for freshness holders for vegetables and others and for water holders. The invention also relates to an absorbent article formed with the absorbent composite, and to a nozzle to be used in a method for producing the absorbent composite.

### BACKGROUND ART

At present, almost all commercially-available absorbent polymers are powdery. For use for sanitary materials such as sanitary napkins and paper diapers, the polymer must be uniformly dispersed on a substrate such as tissue, nonwoven fabric, cotton. The recent tendency in the art is toward using a large amount of an absorbent polymer for the purpose of increasing the absorbent capacity, which, however, may often bring about some problems in that, owing to vibration during transportation or use thereof, a part of the absorbent polymer may locally concentrate or may move after absorption. Given that situation, there are known various absorbent materials such as those mentioned below, but absorbent polymers having higher stability and having higher absorbing speed are desired.

An absorbent material that comprises fibers buried in an absorbent polymer has been proposed (see JP-B-8-19609). This is obtained by mixing or kneading fibers with an absorbent polymer swollen with water or the like, thereby making the fibers buried in the polymer, then drying and grinding it. However, in the absorbent material, the non-swelling fibers are buried in the swelling absorbent polymer, and therefore the absorbent material may often bring about some problems in that it may be troubled by swelling failure to lower its absorbent capacity and absorbing speed and that, after absorption, the absorbent material may warp and the absorbent gel may drop off from the substrate. In particular, while an absorbent gel and fibers are mixed and kneaded, the polymer chains may be cut with the result that the absorbent material could not have its intrinsic absorbent capacity, and in addition, a part of short fibers may be completely buried in the absorbent polymer, therefore failing to express the liquid conductivity that the fibers have but exclusively causing swelling failure. Further, since the absorbent material has a squarish surface, it may irritate skins. In addition, when the absorbent material is pressed so as to thin it, it may cause other problems in that the absorbent polymer therein may be broken and its debris may leak out from the absorbent article and that, since the bonding force between the absorbent polymer and fibers is weak, the fibers and the absorbent gel may be separated while swollen and the absorbent gel may move on the fibers.

An absorbent material that comprises fibers bonded to the surface of an absorbent polymer has been proposed (see JP-A-58-163438). This is obtained by spraying a fiber melt onto a particulate absorbent polymer and mixing them. In this, however, since the bonding force of the fiber melt is weak and since the swelling absorbent polymer bonds to a non-swelling substrate, there may occur some problems in that the absorbent material may warp after absorption and that the absorbent gel may drop off from the substrate in an amount of 50% by weight or more.

An absorbent material that comprises a web-like absorbent polymer bound to a fibrous substrate has been proposed. This may be obtained by spraying an aqueous solution containing a material to give an absorbent polymer onto a fibrous substrate, and polymerizing it on the substrate (see JP-A-60-149609). In the absorbent material, the absorbent polymer hardly drops off from the substrate, but the material may readily face swelling failure and its absorbing speed is low.

An absorbent material with a particulate absorbent polymer bound to the surface of a fibrous substrate has been proposed. This may be obtained by droplet polymerization that comprises spraying an aqueous solution containing a material to give an absorbent polymer onto a fibrous substrate and polymerizing it during the spraying (see JP-A-2000-328456). The absorbent material may hardly undergo swelling failure and its absorbing speed is high, in which, however, the absorbent polymer may readily drop off from the substrate.

A method has been proposed, which comprises spraying an aqueous solution containing a material to give an absorbent polymer, onto a raised fibrous substrate, and polymerizing it on the substrate to thereby make the absorbent polymer bound to the fibrous substrate (JP-A-2004-91996). In this, droplets of an aqueous solution that contains a material to give an absorbent polymer are sprayed onto a raised fibrous substrate whereby the droplets are discontinuously disposed on the substrate and polymerized to produce an absorbent material. In the absorbent composite produced according to this method, the absorbent polymer particles are spherically held by the fibrous substrate. Specifically, the absorbent polymer does not form a mass, but its particles are individually fixed on the substrate. Accordingly, this may undergo swelling obstruction by fibers and its swelling speed may be larger than 100 seconds.

### DISCLOSURE OF THE INVENTION

### Problems that the Invention is to Solve

The invention has been made in consideration of the above-mentioned problems. Concretely, the invention is to provide an absorbent composite having a large absorption and a high absorbing speed and having suitable softness, in which the absorbent polymer is kept uniformly dispersed on the support throughout before and after absorption, and to provide a method for efficiently producing the composite.

### Means for Solving the Problems

We, the present inventors have assiduously studied for solving the above problems. As a result, we have found that an absorbent composite with an absorbent polymer having a specific morphology bound to a substrate can solve the above problems, and have achieved the invention.

Specifically, the subject matter of the invention resides in an absorbent composite comprising a bound particulate absorbent polymer in which two or more nearly spherical absorbent polymer particles are bound to each other and a web-like absorbent polymer, wherein the bound particulate absorbent polymer and the web-like absorbent polymer are bound to a substrate.

The invention also resides in a method for producing an absorbent composite, which comprises polymerizing a polymerizable monomer to give an absorbent polymer and a polymerization initiator in a vapor phase in a mode of droplet polymerization, and contacting it with a substrate while the conversion of the polymerizable monomer is at most 40%.

The invention also resides in an absorbent article formed with the absorbent composite, especially to a sanitary material (in particular, diapers), an industrial material and an agricultural material.

The invention also resides in a polymerization nozzle for use in a method of producing an absorbent composite that comprises polymerizing a polymerizable monomer to give an absorbent polymer and a polymerization initiator in a vapor phase in a mode of droplet polymerization, and contacting it with a substrate while the conversion of the polymerizable monomer is at most 40%, in which the wall thickness of the tip to constitute the nozzle orifice is at most 10 mm. Especially preferably, the nozzle is provided with a mechanism capable of forming a liquid film by continuously or intermittently applying a liquid toward the nozzle orifice of the polymerization nozzle, from outside the nozzle orifice.

### EFFECT OF THE INVENTION

The absorbent composite of the invention has a large absorption and a high absorbing speed and has suitable softness, in which an absorbent polymer is kept uniformly dispersed on the substrate throughout before and after absorption. Accordingly, it is suitable for sanitary materials such as paper diapers and sanitary protections, industrial materials for waste water absorption and holding, and agricultural materials for freshness holders for vegetables and others and for water holders. According to the method for producing an absorbent composite of the invention, the absorbent composite having the characteristics as above can be efficiently produced. Using the nozzle of the invention enables more efficient production of the absorbent composite.

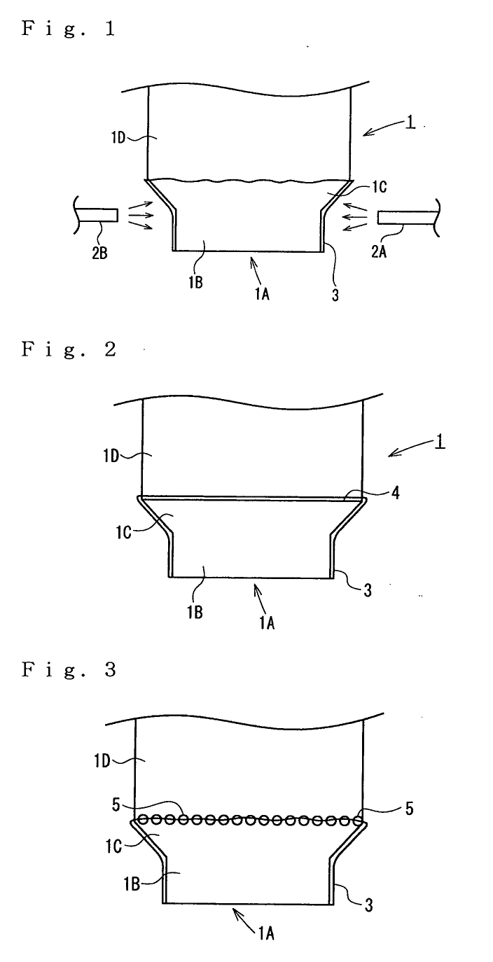

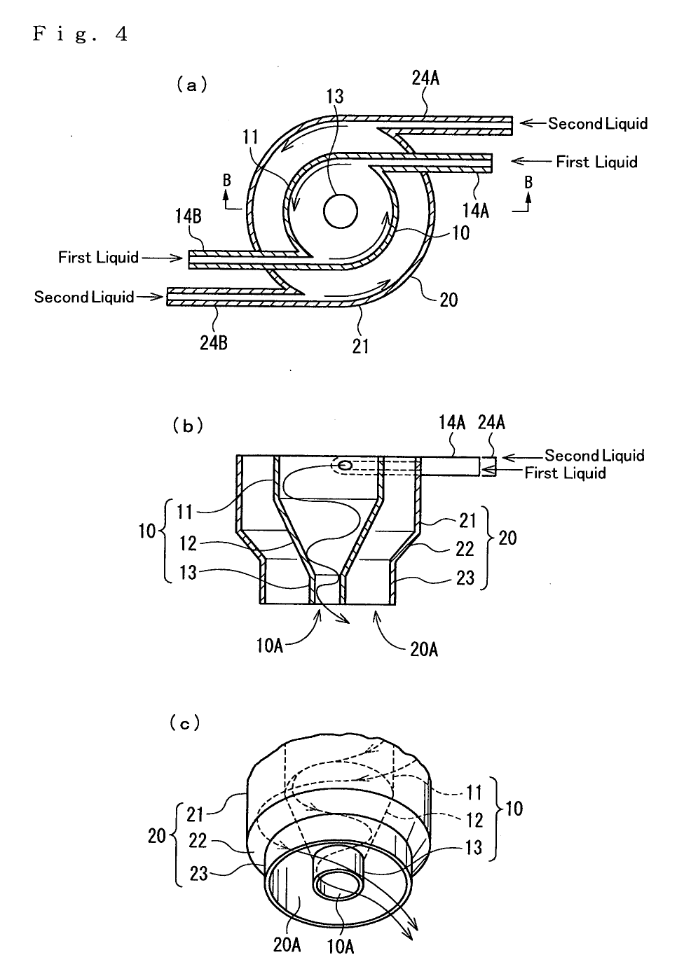

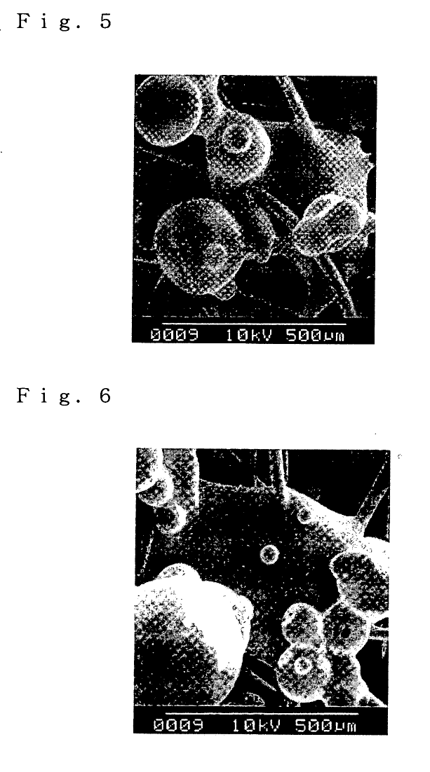

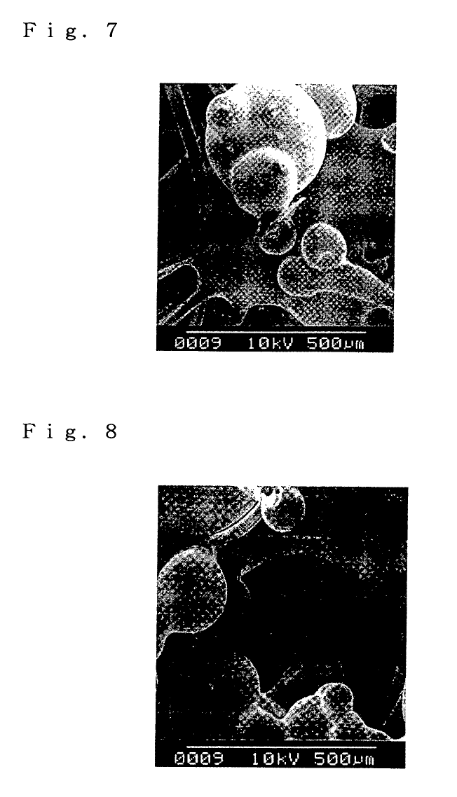

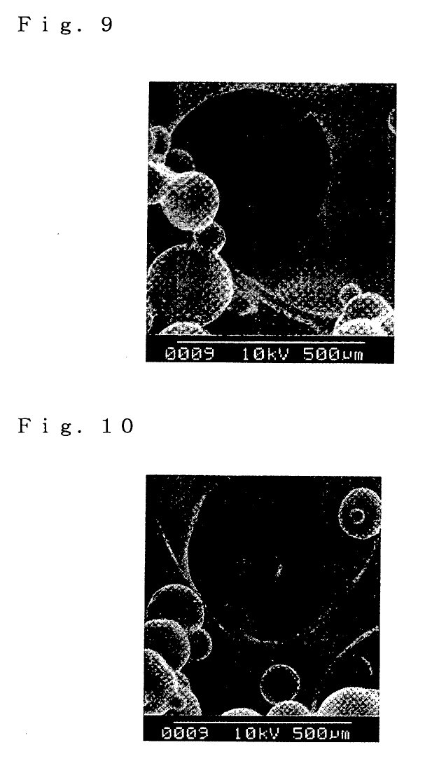

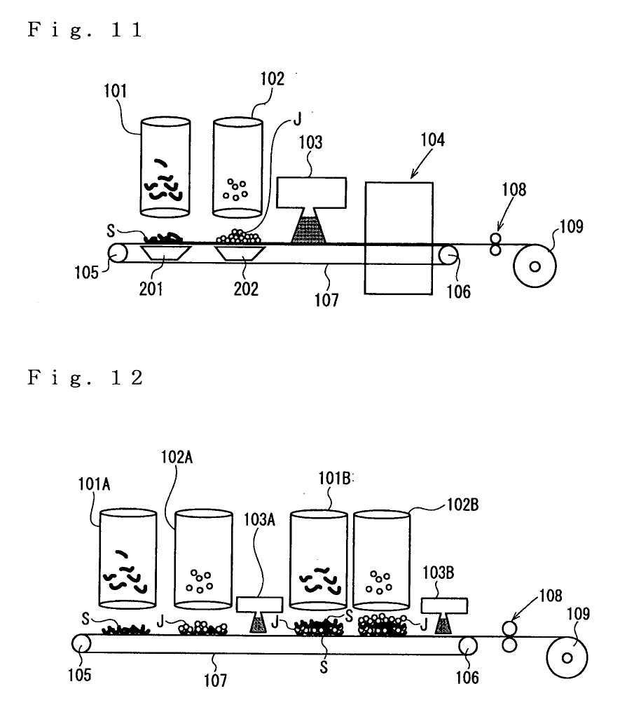

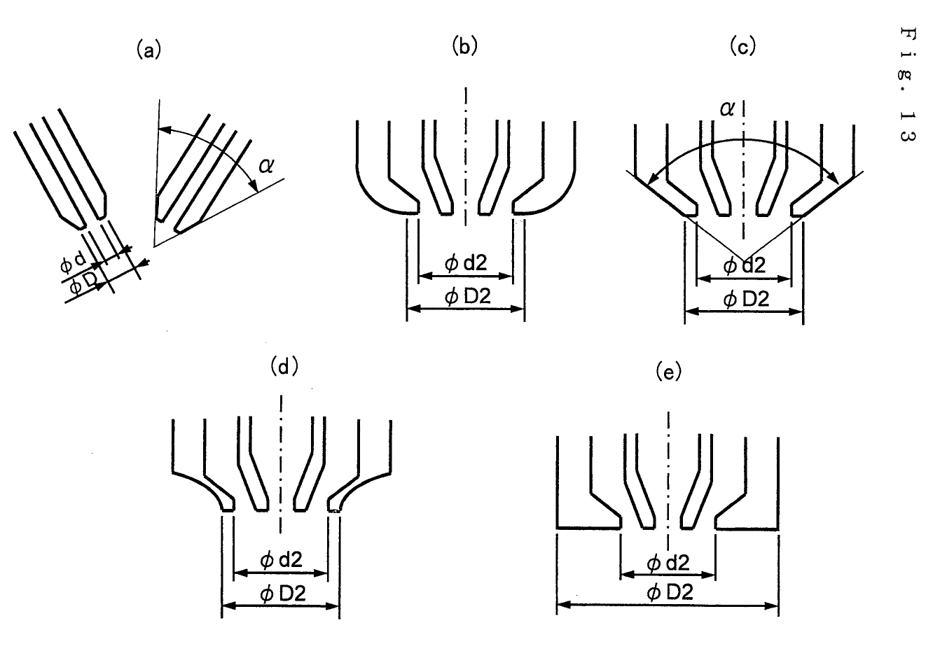

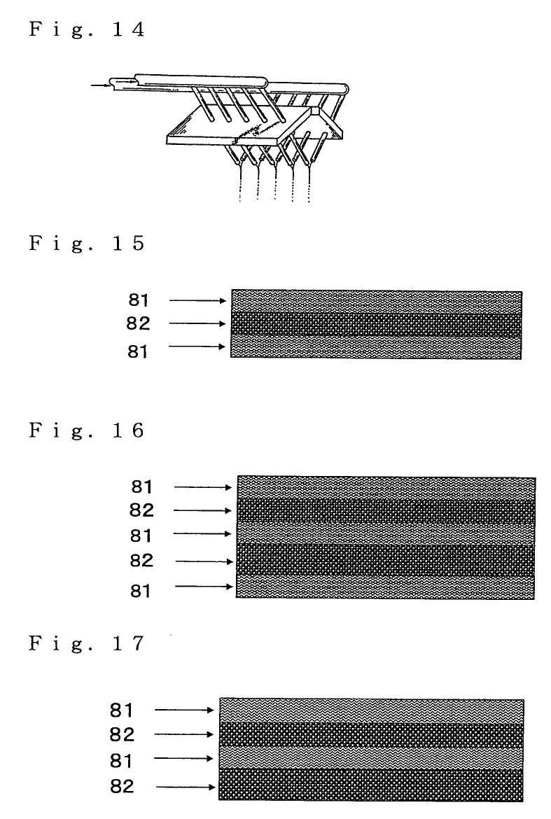

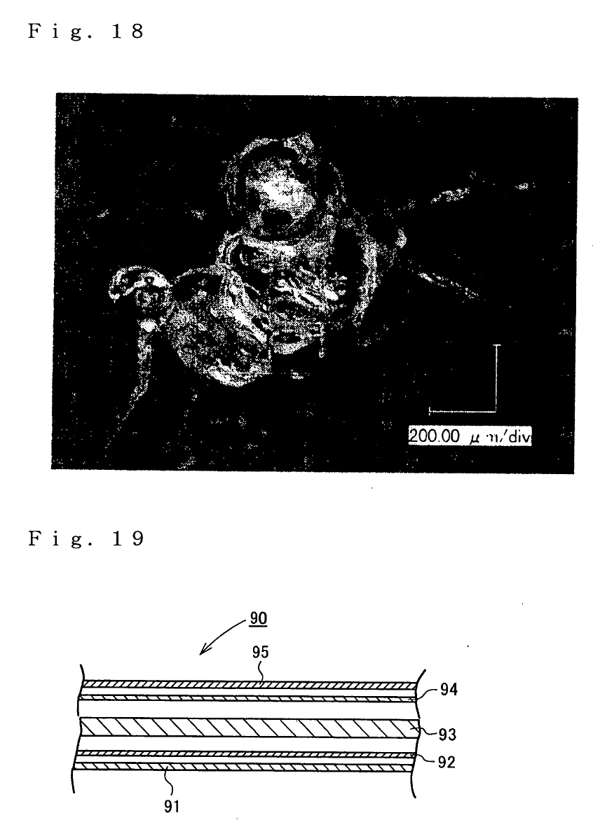

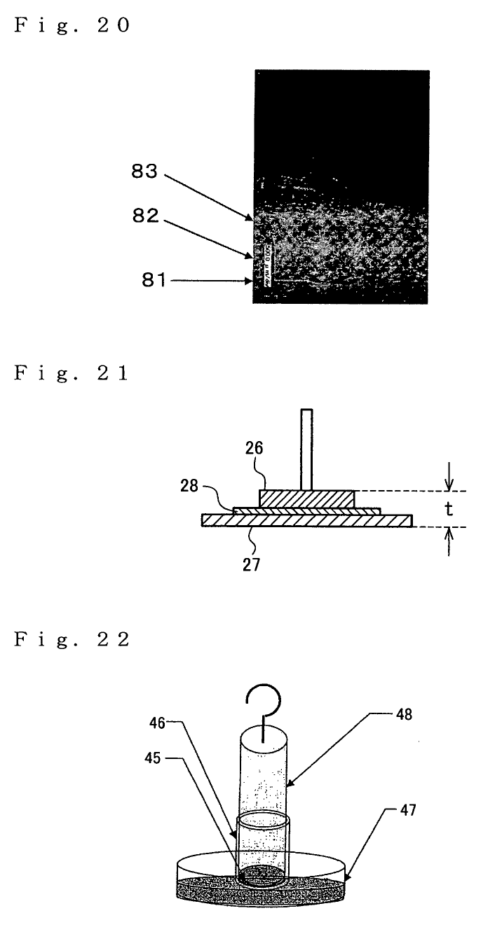

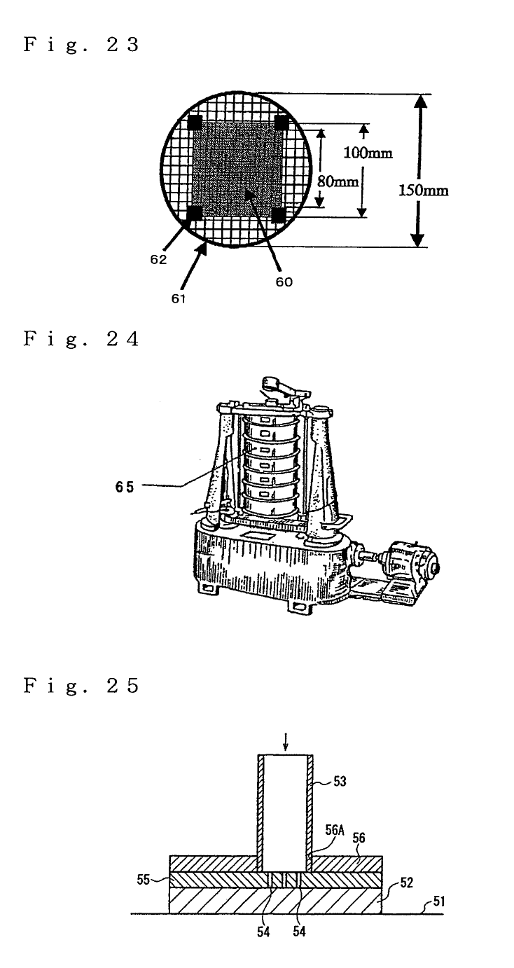

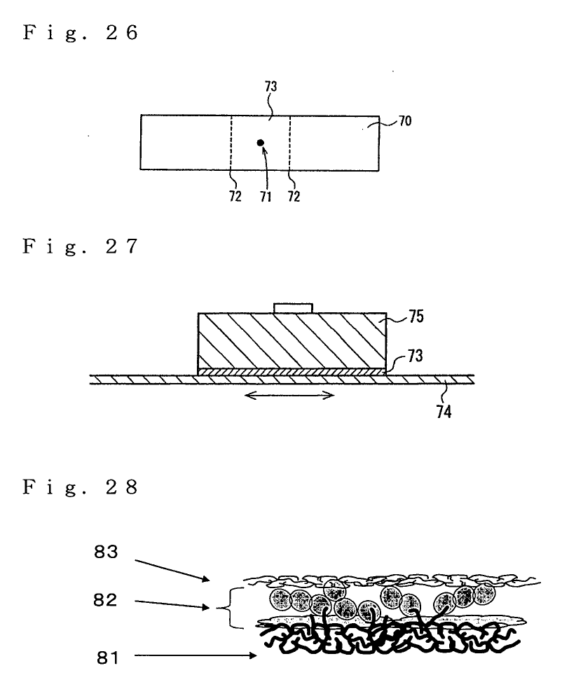

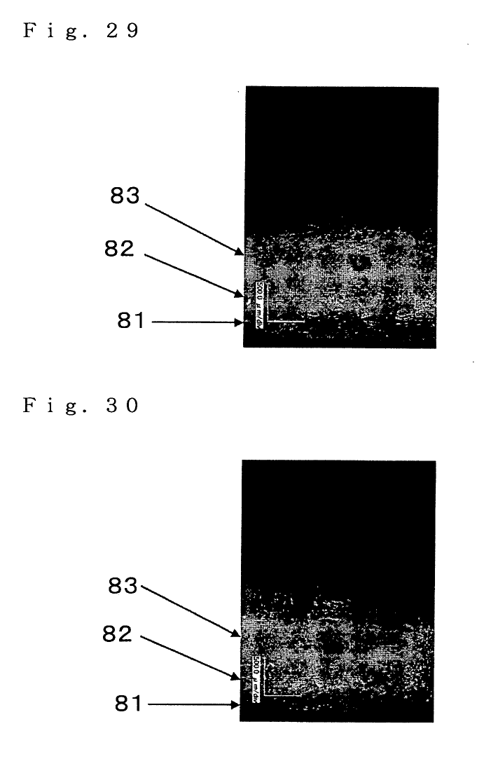

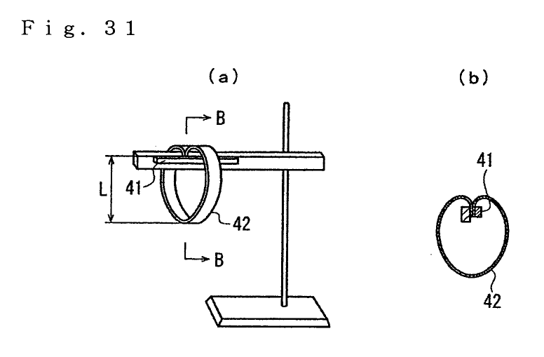

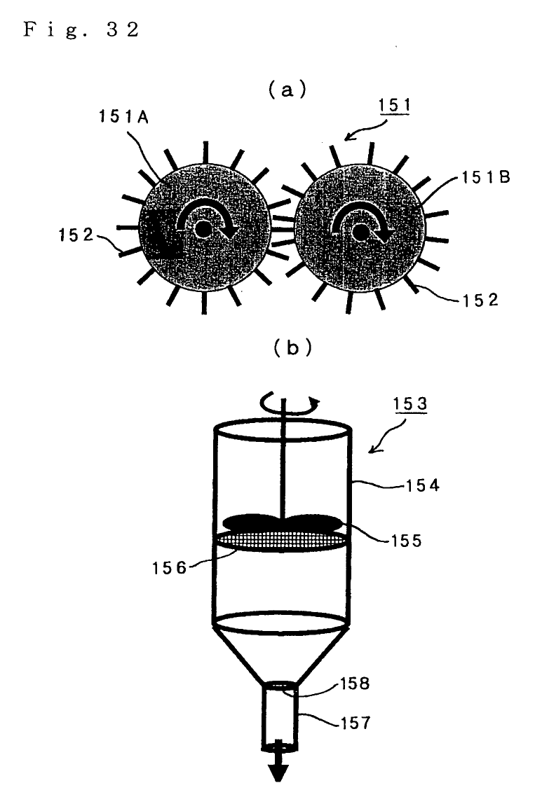

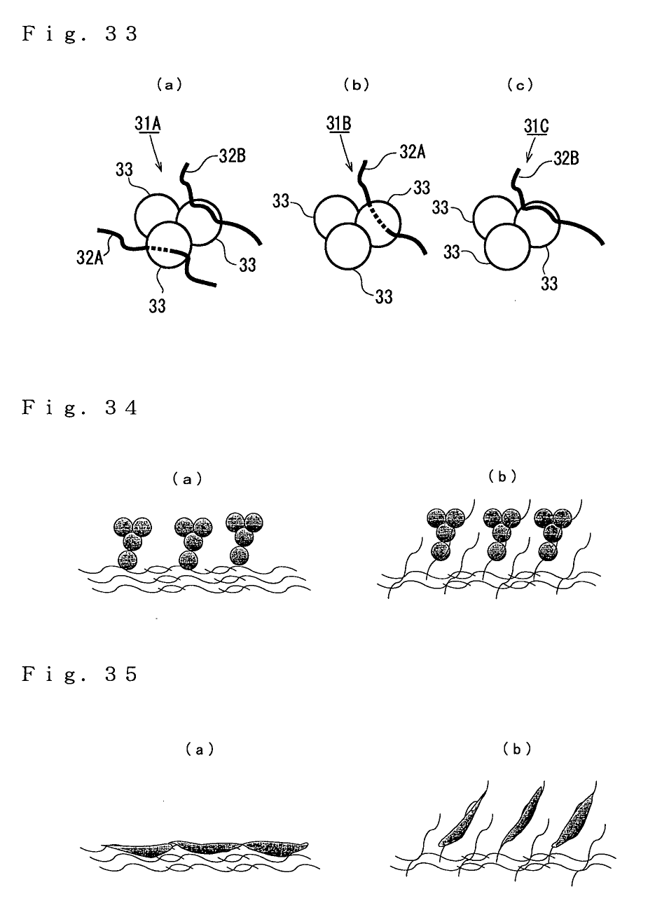

### BRIEF DESCRIPTION OF THE DRAWINGS

[FIG. 1] It is a front view of a spray nozzle, showing an embodiment of a polymerization method and a polymerization device of the invention.

[FIG. 2] It is a front view of a spray nozzle, showing another embodiment of a polymerization method and a polymerization device of the invention.

[FIG. 3] It is a front view of a spray nozzle, showing still another embodiment of a polymerization method and a polymerization device of the invention.

[FIG. 4] It shows a double concentric centrifugal jet nozzle suitable to the invention, in which (a) is a horizontal cross-sectional view of the upper inlet duct part of the nozzle, (b) is a vertical cross-sectional view thereof, and (c) is a perspective view thereof.

[FIG. 5] It is a scanning electromicroscopic (SEM) picture of an absorbent composite of Example 1.

[FIG. 6] It is a scanning electromicroscopic (SEM) picture of an absorbent composite of Example 2.

[FIG. 7] It is a scanning electromicroscopic (SEM) picture of an absorbent composite of Example 3.

[FIG. 8] It is a scanning electromicroscopic (SEM) picture of an absorbent composite of Example 4.

[FIG. 9] It is a scanning electromicroscopic (SEM) picture of an absorbent composite of Example 5.

[FIG. 10] It is a scanning electromicroscopic (SEM) picture of an absorbent composite of Example 6.

[FIG. 11] It is a schematic explanatory view showing one example of a production step of an absorbent composite of the invention.

[FIG. 12] It is a schematic explanatory view showing another example of a production step of an absorbent composite of the invention.

[FIG. 13] It is a cross-sectional view showing a method of computation of the wall thickness of a tip part of constituting a nozzle orifice.

[FIG. 14] It is a perspective view showing a nozzle unit used in Examples and Comparative Examples.

[FIG. 15] It is a cross-sectional view showing the layer constitution of an absorbent composite of Example 7.

[FIG. 16] It is a cross-sectional view showing the layer constitution of an absorbent composite of Example 10.

[FIG. 17] It is a cross-sectional view showing the layer constitution of an absorbent composite of Example 11.

[FIG. 18] It is a scanning electromicroscopic picture of an absorbent composite of Example 1.

[FIG. 19] It is a horizontal cross-sectional view showing the constitution of an absorbent article produced in Examples 19 and 23.

[FIG. 20] It is a digital photomicroscopic picture of the cross section of an absorbent composite of Example 1.

[FIG. 21] It is a cross-sectional view showing a thickness gauge.

[FIG. 22] It is a schematic view showing a device for measuring an absorbent capacity under pressure.

[FIG. 23] It shows a relational position of a sample in measuring the absorbent polymer dropout percentage thereof.

[FIG. 24] It is a perspective view showing a ro-tap shaker.

[FIG. 25] It is a cross-sectional view of a device for measuring a gel dropout percentage, and an absorbing speed and a desorption of an absorbent article.

[FIG. 26] It is a top view showing a cut part of a sample.

[FIG. 27] It is a cross-sectional view showing a shaken state in measuring a gel dropout percentage.

[FIG. 28] It is a schematic cross-sectional view of a compression-shaped absorbent composite.

[FIG. 29] It is a digital photomicroscopic picture of the cross section of an absorbent composite of Example 2.

[FIG. 30] It is a digital photomicroscopic picture of the cross section of an absorbent composite of Example 3.

[FIG. 31] It shows a perspective view (a) of a device for measuring a bending resistance of a sample, and a shape (b) of the sample to be fitted to the device.

[FIG. 32] It is a graphical view (a) showing an outward appearance of an opening device used for evaluation of openability, and a graphical view (b) showing an outward appearance of a screening device for it.

[FIG. 33] It is a schematic explanatory view of grouping the binding condition of an absorbent polymer and fibers.

[FIG. 34] It is a schematic explanatory view showing a condition of a bound particulate absorbent polymer bound to a substrate.

[FIG. 35] It is a schematic explanatory view showing a condition of a web-like absorbent polymer bound to a substrate.

### DESCRIPTION OF REFERENCE NUMERALS AND SIGNS

1 Spray nozzle
2A, 2B Protective liquid spray nozzle
3 Protective liquid film
4 Protective liquid jet-out slit
5 Protective liquid jet-out orifice
10 First nozzle
10A Nozzle orifice
11 Liquid inlet part
12 Taper part
13 Liquid jet-out part
14A, 14B Inlet duct
20 Second nozzle
20A Nozzle orifice
21 Liquid inlet part
22 Taper part
23 Liquid jet-out part
24A, 24B Inlet duct
26 Adaptor
27 Sample bed
28 Sample
31A, 31B, 31C Absorbent composite
32A Partially-buried fibers
32B Surface-adhering fibers
33 Absorbent polymer particles
41 Grip
42 Sample piece
45 Sample disc for measurement
46 Cylinder with wire net
47 Laboratory dish
48 Weight
51 Smooth surface
52 Absorbent composite or diaper
53 Cylinder
54 Through-hole
55 Acrylic plate
56 Disc plate
60 Absorbent composite
61 Standard sieve
62 Tape
65 Ro-tap shaker
70 Absorbent composite
71 Center
72 Cut-off line
73 Sample
74 Acrylic plate
75 Weight
81 Substrate
82 Absorbent polymer
83 Fluff pulp
90 Absorbent article
91 Liquid-impervious sheet
92 Tissue
93 Absorbent composite
94 Tissue
95 Liquid-pervious fibrous material
101, 101A, 101B Non-shaped fiber deposition tower
102, 102A, 102B Polymerization tower
103, 103A, 103B Surface-treatment agent spray
104 Dryer tower
108 Press roller
109 Winder
121 Liquid-impervious sheet
122 Tissue
124 Absorbent composite
125 Tissue
126 Liquid-impervious fibrous material
151 Opening device
152 Pin
153 Screening device
154 Cylinder
155 Stirring blade
156 Metal net
157 Discharge duct part
158 Metal net
201 Aspirator
S Fibers

### MODE FOR CARRYING OUT THE INVENTION

The absorbent composite and its production method of the invention are described in detail hereinunder. The description of the constitutive elements of the invention given hereinunder is for some typical embodiments of the invention, to which, however, the invention should not be limited. In this description, the numerical range expressed by the wording “a number to another number” means the range that falls between the former number indicating the lowermost limit of the range and the latter number indicating the uppermost limit thereof.

The absorbent composite of the invention is characterized in that it comprises, bound to a substrate, a bound particulate absorbent polymer of two or more, nearly spherical absorbent polymers bound to each other and a web-like absorbent polymer.

### I. Absorbent Polymer

The absorbent polymer to be used in the absorbent composite of the invention plays a role of absorbing liquid such as water, urine, blood, in accordance with its use and object. The absorbent polymer is generally produced through droplet polymerization. Droplet polymerization means a mode of polymerization that comprises contacting a polymerizable monomer and a polymerization initiator and polymerizing the monomer while it is in droplets. During polymerization, the system may contain any other ingredient than the polymerizable monomer and the polymerization initiator, not significantly detracting from the polymerization.

### (Polymerizable Monomer)

Not specifically defined in point of its type, the polymerizable monomer may be any one capable of giving an absorbent polymer. Preferably, its polymerization initiation temperature is relatively low (generally, 70° C. or lower) and its polymerization may start with a redox-type initiator. Also preferably, the monomer is soluble in water. One typical example of the polymerizable monomer of the type suitable to the invention is an aliphatic unsaturated carboxylic acid. The carboxylic acid may be in the form of its salt so far as the salt may easily undergo ionic dissociation. In this description, the aliphatic unsaturated carboxylic acid for use in the invention should include its salt. The absorbent polymer that is nearer to neutral is more stable to human bodies such as skins and mucous membranes. Accordingly, the polymerizable monomer is preferably a mixture of an acid and a salt. Preferred examples are unsaturated monocarboxylic acids such as acrylic acid, methacrylic acid; and unsaturated dicarboxylic acids such as maleic acid, itaconic acid. Of those, more preferred are acrylic acid and methacrylic acid as giving polymers having a higher absorption; even more preferred is acrylic acid.

In case where the polymerizable monomer is a salt, it is generally a water-soluble salt such as alkali metal salts, alkaline earth metal salt, ammonium salts. The degree of neutralization of the salt may be suitably determined, depending on the object of the invention. The absorption of the absorbent polymer tends to be higher when the degree of partial neutralization of the aliphatic unsaturated carboxylic acid monomer for the polymer is more than a certain degree. Accordingly, for example, in case of acrylic acid, it is desirable that from 20 to 90 mol % of the carboxyl group of the acid is neutralized with an alkali metal salt or an ammonium salt. For neutralization of the aliphatic unsaturated carboxylic acid monomer, usable is an alkali metal hydroxide or bicarbonate, or ammonium hydroxide. Of those, preferred is an alkali metal hydroxide such as sodium hydroxide or potassium hydroxide since the ion dissociability of the resulting salt is good and since the resulting polymer may have a high absorption.

One or more polymerizable monomers may be used herein. Preferably, the essential ingredient of the polymerizable monomer is one capable of giving an absorbent polymer through polymerization. More preferably, the polymerizable monomer comprises the above-mentioned aliphatic unsaturated carboxylic acid as the essential ingredient thereof. “Essential ingredient” as referred to herein means that the aliphatic unsaturated carboxylic acid accounts for at least 50 mol %, preferably at least 80 mol % of the overall amount of the polymerizable monomer. The other polymerizable monomer than the above-mentioned aliphatic unsaturated carboxylic acid may be a polymerizable monomer copolymerizable with the above-mentioned aliphatic unsaturated carboxylic acid, including, for example, water-soluble monomers such as (meth)acrylamide, poly(ethylene glycol) (meth)acrylate, 2-hydroxyethyl (meth)acrylate; alkyl acrylates such as methyl acrylate, ethyl acrylate. The solubility in water of alkyl acrylates is not so high. The ester maybe copolymerized within a range not detracting from the performance of the resulting absorbent polymer. “(Meth)acryl” as referred to in this description means both “acryl” and “methacryl”.

The polymerizable monomer is generally used in the form of its aqueous solution. The lowermost limit of the concentration of the aqueous solution is preferably 20% by weight since the absorption of the absorbent polymer after polymerization may be large, more preferably 25% by weight. Its uppermost limit is 80% by weight from the viewpoint of easy handlability of the polymerization liquid.

### (Polymerization Initiator)

The polymerization initiator to be used in producing the absorbent polymer for the absorbent composite of the invention may be any one usable in polymerization of the above-mentioned polymerizable monomer into an absorbent polymer, and its type is not specifically defined. Since the polymerizable monomer is used generally in the form of its aqueous solution, the polymerization initiator is preferably used also in the form of its aqueous solution. The polymerization may be either successive polymerization or chain polymerization. Preferred is radical polymerization of chain reaction since the radical polymerizability of the monomer used is high. Accordingly, the polymerization initiator is preferably a radical polymerization initiator.

The initiator maybe a water-soluble radical polymerization initiator, including, for example, peroxides and azo compounds. The peroxides may be inorganic or organic ones, concretely including ammonium, alkali metal or potassium persulfates; hydrogen peroxide; t-butyl peroxide, acetyl peroxide. The azo compounds include water-soluble 2,2′-azobis(2-amidinopropane)-dihydrochloride.

Radical polymerization is initiated by decomposition of a polymerization initiator. The decomposition of a polymerization initiator is generally thermal decomposition. Thermal decomposition includes a case where a polymerizable monomer heated up to a temperature not lower than the decomposition temperature of a polymerization initiator is contacted with an unheated polymerization initiator to initiate polymerization of the monomer.

The polymerization initiator may be grouped into a unary one and a binary one. A binary one is preferred to a unary one since it hardly causes clogging of a reaction nozzle and since its polymerization speed is high. One typical example of the binary polymerization initiator is a redox-type polymerization initiator. The redox-type polymerization initiator is a system that produces an initiator species effective for radical polymerization, through contact of an oxidizing agent and a reducing agent therein (see Chemistry of Polymer Synthesis, Takayuki Ohtsu, Kagaku Dojin, 1979, pp. 66-69).

The oxidizing agent includes, for example, peroxides such as hydrogen peroxide, t-butyl hydroperoxide, cumene hydroperoxide; persulfates such as ammonium persulfate, potassium persulfate; ceric salts; permanganates; chlorites; hypochlorites; and other inorganic salts. Of those, preferred is hydrogen peroxide as it is stable at room temperature (in this description, room temperature is generally from 15 to 25° C.). The amount of the oxidizing agent to be used may be generally from 0.01 to 10% by weight of the polymerizable monomer, preferably from 0.1 to 2% by weight.

The reducing agent is preferably one miscible with the oxidizing agent to form a redox system. Concretely, it may be a water-soluble reducing agent including sulfites such as sodium sulfite, sodium hydrogensulfite; sodium thiosulfate; cobalt acetate; copper sulfate; ferrous sulfate; L-ascorbic acid; alkali metal L-ascorbates. Above all, preferred are L-ascorbic acid and alkali metal L-ascorbates that are stable at room temperature. The amount of the reducing agent to be used may be generally from 0.001 to 10% by weight of the polymerizable monomer, preferably from 0.01 to 2% by weight.

### (Crosslinking Agent)

For increasing the absorption of the absorbent polymer to be produced, a crosslinking agent may be used in polymerization to produce the absorbent polymer. The crosslinking agent is preferably a polyvinyl compound (polyfunctional compound) copolymerizable with the above-mentioned polymerizable monomer, and a water-soluble compound having plural functional groups reactive with a carboxylic acid. The polyvinyl compound includes, for example, N,N′-methylenebis(meth)acrylamide, polyalcohol poly(meth)acrylates. The water-soluble compound having plural functional groups reactive with a carboxylic acid includes polyglycidyl ethers such as ethylene glycol diglycidyl ether, polyethylene glycol diglycidyl ether. Of those, especially preferred are N,N′-methylenebis(meth)acrylamide and polyethylene glycol diacrylate as they are soluble in water. The amount of the crosslinking agent to be used may be generally from 0.001 to 1% by weight of the amount of the polymerizable monomer, preferably from 0.01 to 0.5% by weight. An aliphatic unsaturated carboxylic acid, especially acrylic acid is self-crosslinkable by itself, but it may be combined with a crosslinking agent so as to more positively form a crosslinked structure.

### (Droplet Polymerization Method)

The absorbent polymer to be used in the absorbent composite of the invention is preferably produced through droplet polymerization from an aqueous solution of a polymerizable monomer and a redox-type polymerization initiator. More preferably, an aqueous solution containing any one of an oxidizing agent and a reducing agent that constitute a redox-type polymerization initiator, and a polymerizable monomer (first liquid), and an aqueous solution containing the other of the oxidizing agent or the reducing agent to constitute the polymerization initiator (second liquid) are contacted with each other in a vapor phase, thereby initiating the polymerization. In this, the polymerizable monomer may also be in the second liquid. The temperature of the first liquid and that of the second liquid are, in general, independently from room temperature to 60° C., preferably from room temperature to 40° C.

One concrete method comprises, for example, making the first liquid and the second liquid individually flow out through the respective nozzles, thereby making the resulting two liquid flow columns collide with each other. The crossing angle at which the liquid flows from the respective nozzles cross each other may be suitably determined depending on the properties of the polymerizable monomer and the flow rate of liquids. In general, when the linear velocity of the liquids is larger, then the crossing angle may be smaller. Since a part of the energy necessary for jetting out the liquids through the nozzles may be utilized for mixing the liquid flows, the crossing angle of the jetting liquid flows is preferably at least 15 degrees. The liquids that are in the form of liquid columns may soon separate into droplets, in which the polymerization may go on further. The diameter dd of the droplets may be calculated according to the following formula, based on the mean diameter dp of the absorbent polymer mass to constitute an absorbent composite and the monomer concentration (total concentration of acrylic acid and sodium acrylate) Cm:

dd=dp/(Cm)^{1/3}.

The lowermost limit of the diameter of the droplets may be generally 5 μm, preferably 50 μm; and the uppermost limit thereof may be generally 3,000 μm, preferably 1,000 μm. The space density of the droplets in the reactor may be estimated from the space capacity of the reaction field, the monomer amount fed to the reactor and the dropping speed of the droplets. The space density of the droplets in the reactor is preferably from 10 to 10,000 g/m³ as reducing the absorbent polymer not in contact with the substrate and reducing the substrate not in contact with the absorbent polymer and as improving the relative yield of the absorbent composite.

For the droplet polymerization, preferred is an atmosphere inert to the polymerization reaction. Concretely, the inert atmosphere includes inert gas such as nitrogen, helium; and carbon dioxide and air. The moisture in the atmosphere is not specifically defined, including a case of water vapor alone. The moisture range in the atmosphere is preferably such that water in the aqueous solution of the polymerizable monomer does not evaporate away before polymerization and that the polymerizable monomer does not precipitate out to lower the polymerization speed or to stop the reaction. The temperature of the polymerization atmosphere may be generally from room temperature to 150° C., preferably not higher than 100° C. The atmosphere may be any of a closed system, an open system or a flow system, but is preferably a flow system for controlling the accompanying flow to occur along with the dropping droplets. Regarding its direction, the flow may be a parallel flow or a countercurrent flow relative to the traveling direction of the liquid column and the droplets. For increasing the conversion of the polymerizable monomer and for increasing the viscosity of the droplets, the flow is preferably a countercurrent flow (in the antigravity direction) so as to prolong the residence time of the droplets in a vapor phase.

### (Structure of Nozzle for Droplet Polymerization)

The polymerization nozzle for producing the absorbent composite of the invention is preferably one that facilitates the above-mentioned droplet polymerization method. Concretely, not specifically defined, the nozzle may be any one through which two material solutions of a polymerizable monomer can be jetted out in a vapor phase, in which the solutions can collide with each other as droplets and can be thereby mixed. Concretely, for example, herein employable are a nozzle unit that comprises nozzles facing to each other as in FIG. 14 (see Japanese Patent 3145156); a slit-type nozzle (see JP-A-11-49805); a nozzle through which at least one of two material solutions of a polymerizable monomer can be jetted out as a liquid film expanding in space in such a manner that the cross section of the jetting liquid film can contain a curved profile or can form a hollow circle (see JP-A-2003-113203); and a double concentric centrifugal jet nozzle. Of those, preferred is a nozzle through which at least one of two material solutions of a polymerizable monomer can be jetted out as a liquid film expanding in space; and more preferred is a double concentric centrifugal jet nozzle.

A typical structure of a double concentric centrifugal jet nozzle is described in detail hereinunder (see FIG. 4). FIG. 4(a) shows the upper inlet duct part of the nozzle. FIG. 4(a) is a horizontal cross-sectional view; FIG. 4(b) is a vertical cross-sectional view; and FIG. 4(c) is a perspective view.

The double concentric centrifugal jet nozzle generally comprises a first nozzle 10 for jetting a first liquid and a second nozzle 20 for jetting a second liquid that are disposed concentrically. The first nozzle 10 comprises a liquid inlet part 11 of an upper large-diameter cylindrical part, a liquid jet-out part 13 of a lower small-diameter cylindrical part, and a taper part 12 disposed between these cylindrical parts to be tapered in the downward direction. The liquid inlet part 11 is provided with two inlet ducts 14A and 14B for introducing a first liquid thereinto, opposite to each other in the tangent line direction of the cylindrical part. Like the first nozzle 10, the second nozzle 20 comprises a liquid inlet part 21 of an upper large-diameter cylindrical part, a liquid jet-out part 23 of a lower small-diameter cylindrical part, and a taper part 22 disposed between these cylindrical parts to be tapered in the downward direction, and the liquid inlet part 21 is provided with two inlet ducts 24A and 24B for introducing a second liquid thereinto, opposite to each other in the tangent line direction of the cylindrical part. The first liquid is forcedly introduced into the liquid inlet part 11 through the inlet ducts 14A and 14B. The first liquid runs downward from the liquid inlet part 11 by the centrifugal force and the gravity thereof, forming a spiral trace on the inner wall of the taper part 12, and then reaches the liquid jet-out part 13. Similarly, the second liquid is forcedly introduced into the liquid inlet part 21 through the inlet ducts 24A and 24B, then runs downward from the liquid inlet part 21 by the centrifugal force and the gravity thereof, forming a spiral trace on the inner wall of the taper part 22, thus reaching the liquid jet-out part 23. The first liquid and the second liquid having reached the liquid jet-out parts 13 and 23 are jetted out through the nozzle orifices 10A and 20A, respectively, having a velocity component in the tangent line direction of the nozzles 10 and 20, then collide with each other in a vapor phase and are thus combined and mixed (see FIG. 4(c)).

Preferably, the nozzle to be used in producing the absorbent polymer is such that the wall thickness of the tip that constitutes the nozzle orifice is thin as it is hardly clogged. Concretely, the wall thickness is preferably at most 10 mm, more preferably at most 3 mm, even more preferably at most 1 mm. Still more preferably, the wall thickness of the tip that constitutes the nozzle orifice is at most 0.7 mm, further more preferably at most 0.5 mm. However, it is extremely difficult to reduce the wall thickness to 0.1 mm or less in point of the precision working of the nozzle structure, and when the wall thickness is too small, the nozzle tip could not keep its strength. Accordingly, too much reducing the wall thickness of the nozzle tip is unsuitable.

The wording “the wall thickness of the tip to constitute the nozzle orifice is at most 10 mm” as referred to herein is meant to indicate the thickness of the end of the tip (tip end) that constitutes the nozzle orifice through which a polymerizable monomer liquid is jetted out, and the thickness may be determined by measuring the length from the outer periphery to the inner periphery of the end of the tip. For example, a nozzle having a cross section as in FIG. 13(a) is referred to. The wall thickness of its tip is represented by ((φD−φd)/2) (in which φD indicates the outer diameter of the nozzle at the nozzle tip face, and φd indicates the inner diameter of the nozzle (aperture) at the nozzle tip face). For example, the wall thickness of the tip of the needle-like nozzle shown in Japanese Patent 3145156 can be determined according to the computation formula. The slit nozzle shown in JP-A-11-49805 has a similar cross-sectional profile to the above, and the wall thickness of its tip can be determined according to the same computation formula (In which φD indicates the width dimension of the outer face of the nozzle at the nozzle tip face, and φd indicates the liquid film jet-out width dimension of the nozzle at the nozzle tip face). Other examples of the cross section of double concentric centrifugal jet nozzles are shown in FIGS. 13(b) to (e), in which the wall thickness of the tip part is represented by ((φD2−φd2)/2) (in which φD2 indicates the outer diameter of the outer nozzle at the nozzle tip face, and φd2 indicates the inner diameter of the outer nozzle at the nozzle tip face). The wall thickness of the tip part of the nozzle shown in JP-A-2003-113203 can be determined according to the computation formula.

When a nozzle of which the wall thickness dimension of the tip falls within the above preferred range and the tip wall is therefore thin is used and even when droplets of a polymerizable monomer may adhere to the tip face, then the jet stream running through the nozzle may be readily in contact with the droplets to appropriately wash them away. In a polymerization process in which water vapor is filled in the atmosphere field in a polymerization reactor, a little water vapor may form dew condensing on and adhering to the tip of the nozzle orifice as water droplets, and therefore droplets of a polymerizable monomer may adhere to the nozzle tip, but even in such a case, the water droplets may dilute the monomer concentration to prevent the promotion of polymerization and therefore the polymer may be readily prevented from growing like gum. Since the nozzle of the type has such a self-washing function, it may prevent a polymerizable monomer from being polymerized so much to grow like gum and even like icicles. In addition, it may prevent fine particles from too much scattering owing to the increase in the jetting-out monomer amount and owing to the reduction in the nozzle orifice diameter, and may prevent such fine particles from adhering to the nozzle orifice, and accordingly, the spray nozzle may be prevented from being clogged, therefore contributing toward safe driving and long-term driving of the apparatus.

Not specifically defined, the profile of the nozzle for use in producing the absorbent polymer is preferably tapered toward the nozzle tip part. For example, as in FIG. 13(b) showing a cross-sectional view of a nozzle, the nozzle may be so tapered that its profile line forms a curved line smoothly expanding outside; or as in FIG. 13(d) also showing a cross-sectional view thereof, the nozzle may be so tapered that its profile line forms a curved line smoothly concaving inside. Further as in FIG. 13(c) showing a cross-sectional view thereof, the nozzle may be so tapered that its profile line forms a linearly-tapering straight line. These profiles may be combined to give a nozzle tip for use herein, of which the curvature of the nozzle surface profile is stepwise changed. Preferred for use herein are a tapered nozzle, and a nozzle of which the profile line is so tapered as to form a curved line smoothly concaving inside. More preferred is a nozzle of which the cross-sectional profile is so constituted that the nozzle profile line is so tapered as to concave inside. When the nozzle profile is tapered or concaved inside, then the nozzle orifice can keep an atmospheric space around it. As a result, the friction between the jet streams from the nozzle orifice and the atmosphere around the nozzle orifice and the whirlpools around it may be reduced, and the particles jetted out through the nozzle orifice may be prevented from scattering away, therefore resulting in that a polymerizable monomer may be prevented from adhering to the nozzle surface and around the nozzle orifice, and the operability with the nozzle may be thereby improved. For ensuring the space around the nozzle orifice, it is desirable that the mean radius of curvature of the part at which the nozzle cross-sectional profile begins to be tapered is as large as possible. Concretely, an advantageous planning mode for it is as follows: The radius of curvature at the start of the tapered part is at least 3 mm, more preferably at least 10 mm, even more preferably at least 20 mm, and the tapered part is smoothly connected to the inclined part of the nozzle body to be led to the outermost periphery of the nozzle.

In case where the nozzle for use in producing the absorbent polymer has a tapered part as above, the crossing angle α of the taper is preferably at most 160°, more preferably at most 90°, even more preferably at most 60° (see FIG. 13(c)). Controlling the crossing angle makes it easy to wash away the polymerizable monomer liquid adhering to the nozzle surface from the nozzle tip. The droplets gathering at the tip are blown away in space along with the polymerizable monomer jetted out through the nozzle, and therefore the nozzle of the type may have a self-washing function, not forming gum or not clogging nozzle in long-term operation of the nozzle.

### (Liquid Film on Nozzle for Droplet Polymerization)

In long-term continuous production of the absorbent polymer, it is desirable to form a liquid film on the outer wall surface of the nozzle for polymerization. In general, the polymerization speed in droplet polymerization is high, and the nozzle may be readily clogged by a polymer product in the method. When a liquid film is formed on the outer wall surface of the nozzle around the nozzle orifice, then it may act as a protective liquid film. Therefore, while two material solutions of a polymerizable monomer are made to collide with each other and are thereby mixed and even when they form fine droplets as being thrown away owing to their collision, they may be washed away before adhering to the area around the nozzle orifice, and the nozzle may be prevented from being clogged through polymerization of fine droplets. Accordingly, when a liquid film is formed in that manner, it may be unnecessary to stop the reaction to clean the nozzle, and this is advantageous in point of the production efficiency.

The liquid film is preferably formed by continuously or intermittently applying a liquid through the nozzle orifice. For forming the liquid film, a liquid may be sprayed onto the outer wall surface of the nozzle, especially onto the wall surface of the upstream side above the nozzle orifice; or using a spray nozzle having a slit or hole for jetting out a protection liquid through it, and a protective liquid may be made to run downward on the outer surface of the polymerization nozzle from the slit or the hole. It is desirable that the liquid film for preventing nozzle clogging (hereinafter the nozzle clogging preventing liquid may be referred to as “protective liquid”, and the liquid film may be referred to as “protective liquid film) is formed on the outer surface of the nozzle in the upstream area above the nozzle orifice through which two material solutions of a polymerizable monomer are jetted out and between the position which the liquid jetted out from the nozzle orifice and the droplets being polymerized do not reach and the position of the nozzle orifice. In case where a liquid such as water is directly sprayed onto the nozzle orifice (see U.S. Pat. No. 3,929,291), the sprayed liquid may interfere with stable jetting, collision and mixing of the polymerization liquids, and the first liquid and the second liquid may be mixed at around the nozzle orifice part whereby the nozzle orifice may be much clogged. Accordingly, attention should be paid to the case.

For carrying out the polymerization while the above liquid film is formed, in general, a polymerization nozzle is used which is provided with a mechanism of forming a liquid film on the outer surface of the nozzle. For example, a double concentric centrifugal jet nozzle (see FIG. 4) is referred to. One embodiment is as in FIG. 1, in which protective film spray nozzles 2A and 2B are provided in the vicinity of the protective film-forming surface of the spray nozzle 1, and a protective liquid is sprayed onto the protective liquid film-forming surface of the spray nozzle 1, from the spray nozzles 2A and 2B, thereby forming a protective liquid film 3. Other embodiments are in FIGS. 2 and 3, in which a slit 4 or holes 5 for jetting out a protective liquid are formed in a position above the protective liquid film-forming surface of the spray nozzle 1, and a protective liquid is jetted out through the slit 4 or the holes 5 and made to run downward to form the protective liquid film 3. In case where a protective liquid is jetted out as in FIGS. 2 and 3, the upper large-diameter cylindrical part 1D of the spray nozzle 1 may have a three-layered structure additionally having a space for the protective liquid in the outermost peripheral part thereof.

As in the above, the protective liquid film is formed on the outer wall surface of the spray nozzle in the area between the position which the liquid jetted out from the nozzle orifice and the droplets being polymerized do not reach and the position of the nozzle orifice. For example, in the case of the double concentric centrifugal jet nozzle of FIG. 4, the protective liquid film-forming surface covers the liquid jetting-out part 23 of the second nozzle 20 and the taper part 22 above it. The liquid jetted out from the nozzle orifice and the droplets being polymerized do not almost reach the area of the liquid inlet part 21 above the taper part 22, and therefore, it is desirable that the protective liquid film is formed in the area covering the liquid jet-out part 23 and the taper part 22 above it. Accordingly, in FIG. 1, the protective liquid spray nozzles 2A and 2B are so provided that the protective liquid film 3 can be formed in the area of the lower small-diameter cylindrical part 1B and the taper part 1C above it where the nozzle orifice 1A of the spray nozzle 1 is formed. In FIGS. 2 and 3, the protective liquid jet-out slit 4 and the holes 5 are provided in the boundary between the taper part 1C of the spray nozzle 1 and the upper large-diameter cylindrical part 1D.

### (Protective Liquid)

The protective liquid is preferably one having a high affinity for any one or more of the first liquid, the second liquid and the polymer product obtained through polymerization. For example, a solvent capable of dissolving the polymerizable monomer in the first liquid and/or the second liquid may be used.

In case where the polymerizable monomer is a water-soluble polymerizable monomer, the solvent may be a hydrophilic solvent including water; alcohols such as methanol, ethanol, isopropyl alcohol; ketones such as acetone, methyl ethyl ketone. When the monomer is an oleophilic polymerizable monomer, then the solvent may be an oleophilic solvent including saturated hydrocarbons such as pentane, hexane, cyclohexane, heptane, cycloheptane; and aromatic hydrocarbons such as toluene, xylene. Further, a solvent having a high affinity for the polymerizable monomer to which a polymerization initiator is not added, may also be used. One or more of these solvents may be used. Since many polymerizable monomers for the absorbent polymer are hydrophilic, hydrophilic solvents such as water, alcohols and ketones are preferred among the above-mentioned solvents.

For the protective liquid, also usable is a solvent capable of dissolving or swelling the particulate polymerization product produced through polymerization. For the solvent of the type, usable are the above-mentioned hydrophilic solvents and oleophilic solvents.

The flow rate of the protective liquid and the thickness of the protective liquid film may be such that the polymerizable monomer and the polymer particles do not adhere to and stay in the area around the nozzle orifice, but must be so selected that the flow rate and the film thickness do not interfere with the jetting of the first and second liquids. For example, in case where the protective liquid film is formed on the outer wall surface of the spray nozzle 1 as in FIGS. 1 to 3, the thickness of the protective liquid film may be preferably from 0.01 to 2 mm or so and the flow rate of the liquid may be from 10 to 2000 ml/min or so per m² of the protective liquid film-forming surface, though varying depending on the protective liquid used and on the composition and the flow rate of the first and second liquids to be jetted out.

### (Constitution of Absorbent Polymer)

The absorbent polymer produced through droplet polymerization is generally in the form of a mass having a major diameter of from 100 to 50000 μm. In particular, the lowermost limit of the major diameter of the mass of the absorbent polymer for use in the absorbent composite of the invention is preferably 200 μm, more preferably 300 μm, and the uppermost limit thereof is preferably 5000 μm, more preferably 2000 μm. Preferably, the lowermost limit of the thickness of the polymer mass is 50 μm, more preferably 100 μm, and the uppermost limit thereof is preferably 3000 μm, more preferably 1000 μm. The range not larger than the above uppermost limit is preferred since the polymer mass may have a large number of bonding parts to a substrate and since the polymer hardly drop off from the substrate; and the range not smaller than the above lower limit is preferred since the polymer mass is hardly influenced by swelling failure by fibers. The morphology of the absorbent polymer may be identified generally through electromicroscopic observation, in which the major diameter indicates the longest diameter of the absorbent polymer mass determined with an electronic microscope and the thickness indicates the longest diameter vertical to the major diameter of the mass. Enveloping or adhering to a part of a substrate, the absorbent polymer mass may be bound to the substrate. In this, the substrate and the absorbent polymer mass may bond to each other only at one position but preferably at 2 or more positions since the absorbent polymer will hardly drop from the substrate. Preferably, the absorbent polymer mass envelops a part of the substrate rather than adhering to the substrate, since its bonding to the substrate may be more strong. In case where the absorbent polymer mass envelops a part of the substrate, the fibers may or may not run through the absorbent polymer. The mass morphology may be grouped into (1) a bound particulate structure comprising nearly spherical absorbent particles bound to each other, and (2) a web-like structure.

The bound particulate structure of the above (1) is meant to indicate that nearly spherical absorbent polymer particles are bound to each other. Concretely, the lowermost limit of the number of the bound absorbent polymer particles to form a mass is generally 3, preferably 5; and the uppermost limit thereof is generally 100, preferably 20, more preferably 10.

The individual absorbent polymer particles constituting the bound particulate absorbent polymer are nearly spherical. The nearly spherical morphology means that the particle is in the form of a sphere as a whole, and its surface may be finely roughened, for example, having projections or recesses. The particle may have pores or cracks inside it. Concretely, for example, the particle morphology includes not only a shape of spheres but also other shapes of ovals, rugby balls, peanuts, dumbbells.

The lowermost limit of the particle size of the particles is preferably 10 μm in view of their fixability to fibers, more preferably 50 μm, even more preferably 100 μm, most preferably 200 μm. The uppermost limit of the particle size of the particles is 2000 μm in view of their absorbing speed, more preferably 1000 μm, even more preferably 800 μm, most preferably 500 μm. The particle size may be determined with SEM (scanning electronic microscope).

The shape and the size of the bound particulate absorbent polymer mass are not specifically defined so far as the major diameter thereof falls within the above-mentioned preferred range. As compared with that of the web-like structure to be mentioned hereinunder, the preferred range of the major diameter of the polymer mass is relatively small, and the preferred range of the thickness thereof is relatively large. Concretely, the lowermost limit of the major diameter is preferably 20 μm, more preferably 100 μm, even more preferably 200 μm, most preferably 300 μm; and the uppermost limit thereof is preferably 5000 μm, more preferably 4000 μm, even more preferably 3000 μm. The lowermost limit of the thickness is preferably 50 μm, more preferably 100 μm, even more preferably 200 μm; and the uppermost limit thereof is preferably 3000 μm, more preferably 2000 μm, even more preferably 1000 μm.

The web-like structure of the above (2) means that the absorbent polymer sticks to a substrate. A specific example of the structure is described for easy imagination thereof. This comprises a flat absorbent polymer sticking to a part of plural overlapping substrate fibers (see FIGS. 5 to 10 of Examples described below).

The web-structured absorbent polymer may exist on a substrate in any morphology thereof, so far as its major diameter falls with the above preferred range. Concretely, it may distribute like islands or may form a sea-like continuous layer, or may form a mixture of the two, or may have any other morphology than these. In view of its liquid perviousness, the polymer preferably distributes like islands. The shape and the size of one island and one sea are not specifically defined, so far as their major diameter falls within the above preferred range. Preferably, however, the lowermost limit of the thickness (the longest diameter in the vertical direction to the maximum length including the web part) is 50 μm, more preferably 80 μm, even more preferably 100 μm; and the uppermost limit thereof is preferably 1000 μm, more preferably 800 μm, even more preferably 500 μm. Preferably, the lowermost limit of the major diameter (the maximum length including the web part) of one island in the island structure is 200 μm, more preferably 300 μm, even more preferably 400 μm; and the uppermost limit thereof is preferably 50000 μm, more preferably 30000 μm, even more preferably 20000 μm. The lowermost limit of the major diameter (the maximum length including the web part) of the sea-like structure is preferably 1000 μm, more preferably 2000 μm; and the uppermost limit thereof is preferably 20000 μm, more preferably 10000 μm.

In the case of a sea-like continuous layer, island-like pores may exist in the sea part. The lowermost limit of the pore size in this case is preferably 100 μm in view of the liquid perviousness of the layer, more preferably 200 μm, even more preferably 300 μm. The uppermost limit of the pore size is preferably 5000 μm since the layer is hardly cracked or broken, more preferably 3000 μm, even more preferably 2000 μm. The lowermost limit of the porosity is preferably 10% by area in view of the liquid perviousness of the layer, more preferably 20% by area, even more preferably 30% by area; and the uppermost limit thereof is preferably 80% by area, more preferably 70% by area, even more preferably 60% by area.

The absorbent polymer produced through droplet polymerization contains the structure of the above (1) and (2) generally in an amount of at least 10% by weight, preferably at least 30% by weight, more preferably at least 50% by weight. In case where the degree of polymerization of the absorbent polymer that is in contact with a substrate during polymerization is high, then the proportion of the structure (1) tends to be higher; but when it is low, then the structure (2) tends to be higher. If the absorbent polymer is in contact with a substrate while it is not as yet sufficiently polymerized, then it may form beads.

The substrate to be used in the absorbent composite of the invention is preferably a fibrous substrate, as described hereinunder. When the absorbent polymer is bound to a fibrous substrate, then the fiber length between one absorbent polymer mass and the other absorbent polymer mass is preferably at least 500 μm since it hardly causes swelling failure between the absorbent polymers, more preferably at least 800 μm, even more preferably at least 1000 μm. The uppermost limit of the fiber length is preferably 100000 μm, more preferably 50000 μm, even more preferably 30000 μm.

One example of the bound stage of an absorbent polymer and a substrate in the absorbent composite of the invention is described with reference to FIG. 33, in which three nearly-spherical absorbent polymer particles bound to each other are bound to fibers. This is grouped into the following three patterns. One is such that a part of a substrate is buried in an absorbent polymer and a part thereof is exposed out (hereinafter this substrate may be referred to as “partly buried substrate”—see FIG. 33(b)). Another is such that a part of a substrate adheres to the surface of an absorbent polymer and a part thereof does not adhere to an absorbent polymer (hereinafter this substrate may be referred to as “surface-adhering substrate”—see FIG. 33(c)). The last is a combination of the partly-buried substrate and the surface-adhering substrate (see FIG. 33(a)).

The absorbent composite of the invention generally comprise any one of the above three structures. The absorbent composite of the invention preferably comprises the last pattern, and more preferably, it is formed of only the last pattern. Specifically, the absorbent composite of the invention includes a partly-buried substrate and/or a surface-adhering substrate. However, the absorbent composite of the invention may contain any other fibers than the partly-buried substrate and the surface-adhering fibers, for example, a substrate neither buried in an absorbent polymer nor adhering to it, a substrate completely buried in an absorbent polymer, and a substrate completely adhering to the surface of an absorbent polymer. The other substrate than the partly-buried substrate and the surface-adhering substrate is preferably as small as possible, more preferably in an amount of at least 30% by weight of the overall substrate in the absorbent composite.

Containing both the bound particulate structure (1) and the web-like structure (2), the absorbent polymer that constitutes the absorbent composite of the invention is not specifically defined in point of the proportion of the bound particulate structure (1) to the web-like structure (2) therein. In case where the polymer contains the structures (1) and (2) as combined, the gel dropout percentage and the polymer dropout percentage of the composite may be reduced when the proportion of the web-like structure (2) is large in the composite; but when the proportion of the bound particulate structure (1) is larger in the composite, then the absorbing speed of the composite may be higher. A preferred ratio of the two is generally such that the proportion of the bound particulate absorbent polymer (1) is from 1 to 10000 parts by weight relative to 100 parts by weight of the web-like absorbent polymer (2), more preferably the former is from 5 to 2000 parts by weight, even more preferably from 10 to 1000 parts by weight.

In case where the composite contains both a web-like absorbent polymer and a bound particulate absorbent polymer as combined therein, the positional relationship between the two is not specifically defined. Especially preferred is an embodiment where a web-like absorbent polymer layer (2) is formed on a substrate and a bound particulate absorbent polymer layer (1) formed on the layer (1), since its liquid perviousness and liquid dispersiveness are good. For the same reason, also preferred is an embodiment where a continuous layer of a web-like absorbent polymer (2) is formed on a substrate and a bound particulate absorbent polymer (1) is formed in the pores of the layer (2).

The proportion of the structures (1) and (2) in the absorbent polymer may be determined according to the following method. An absorbent composite is cut into a piece of 5 cm×5 cm square. Using a stainless double-edged knee-shaped clipper scissors (FST 14063-09), the substrate is removed from the sample, and the absorbent polymer is cut out of it. The sample is observed carefully with a digital photomicroscope (Keyence's VH-8000, having a magnification of from 25 to 150), and the absorbent polymer is divided into (1) the bound particulate polymer, (2) the web-like polymer, and the others, using a stainless single-edged bone clipper scissors (FST 14077-10). The weight of each section was determined, and the ratio by weight of the bound particulate polymer (1) to the web-like polymer (2) is computed.

### II. Substrate

### (Effect of Substrate)

In the absorbent composite of the invention, the substrate preferably exhibits an effect of liquid dispersiveness and an effect of liquid perviousness. The liquid diffusiveness means an effect of such that the substrate can rapidly diffuse the liquid applied to the absorbent composite in the horizontal direction of the composite, thereby uniformly distributing it in the absorbent polymer. The liquid perviousness means an effect of such that the substrate can rapidly transmit the liquid applied to the absorbent composite in the vertical direction of the composite, thereby transferring it to the absorbent polymer. In general, the substrate competitively exhibits its liquid-diffusive function and its liquid-pervious function for the liquid applied to the composite in the direction toward the substrate side. In general, the liquid-diffusing speed of the substrate is larger than that of the absorbent polymer, and therefore, the liquid applied to the composite first diffuses rapidly on the substrate and uniformly expands thereon, and thereafter it runs into the absorbent polymer. During the series of the liquid absorption process, the substrate not only has some influence on the absorbing speed but also is effective for preventing a phenomenon of lump formation of such that the liquid is partly localized in the absorbent polymer and therefore the absorbent polymer could not exhibit its complete performance.

### (Type of Substrate)

The substrate that constitutes the absorbent composite of the invention plays a role of fixing an absorbent polymer thereon. Concretely, various types of materials such as plates, sheets, films and fibrous aggregations, and various shapes of substrates may be used herein. As the case may be, while a fibrous substrate is dispersed in a vapor phase, it may be brought into contact with an absorbent polymer being formed through droplet polymerization, and the two may be simultaneously deposited. Since the morphology retentiveness of the substrate may be increased owing to the absorbent polymer binding thereto, the substrate may be formed of a soft and rough material or plate with filaments merely laid thereon.

The surface condition of the substrate is preferably rough in some degree so that an absorbent polymer can be readily bound thereto. Also preferably, the substrate may have open spaces (pores or voids), as it is excellent in point of water perviousness, liquid perviousness, water conductivity and liquid conductivity thereof. From these viewpoints, the substrate is preferably a fibrous substrate formed of an aggregation of fibers. From the viewpoint of effective use or natural resources, for example, the substrate is preferably an unshaped fibrous substrate that may be readily recycled.

In the fibrous substrate, the fibers bound to an absorbent polymer are entangled and stabilized, and therefore, they prevent the absorbent polymer from moving for rotary movement or parallel movement while pressed or vibrated. In particular, the fibers partly buried in the absorbent polymer greatly contribute to the fixation of the absorbent polymer after liquid absorption. In addition, the fibrous substrate exhibits an effect of ensuring suitable liquid paths in the absorbent polymer having swollen through absorption.

The material of the fibrous substrate includes, for example, natural fibers such as vegetable fibers, animal fibers, mineral fibers; and chemical fibers such as regenerated fibers, semi-synthetic fibers, synthetic fibers, mineral fibers. Of those, natural fibers are preferred for use in sanitary materials and the like, since they hardly irritate skins and since they feel soft. Chemical fibers are also preferred in point of their uniformity. (See Handbook of Fibers (edition of raw materials), edited by the Fiber Society of Japan, Maruzen, 1968; Handbook of Fibers (edition of working), edited by the Fiber Society of Japan, Maruzen, 1969; Synthetic Fibers (Hiroshi Sofue, Dai-Nippon Tosho, 1977).)

The fibrous substrate preferably has a chemical affinity for the absorbent polymer, since it may readily bond to the absorbent polymer. In general, the absorbent polymer is hydrophilic. Concretely, therefore, the fibrous material is preferably a water-conductive one (having the property of attracting water toward an absorbent polymer) or a hydrophilic one formed of cellulosic fibers of, for example, pulp, rayon, cotton, regenerated cellulose; or polyamide fibers; or polyvinyl alcohol fibers. Of those, preferred are cellulosic fibers as they hardly irritate skins and as they feel soft. Especially preferred is pulp. Concretely, the pulp is preferably mechanical pulp such as ground wood pulp; chemical-mechanical pulp such as semi-chemical pulp, chemical ground pulp; chemical pulp such as sulfite pulp, sulfate pulp, soda pulp, nitrate pulp, chloride pulp; pulp prepared by mechanically grinding or powdering formed paper; and used paper pulp prepared by mechanically grinding or powdering used paper.

The affinity between the hydrophilic absorbent polymer and the substrate may be determined by measuring the contact angle of water to the substrate. A substrate of which the contact angle to water on the surface of the substrate is smaller may have a higher affinity for an absorbent polymer and may have a higher adhesiveness to the polymer. Accordingly, the contact angle to water on the surface of the substrate is preferably at most 60°, more preferably at most 50°, even more preferably at most 40°. The contact angle varies depending on the shape of the substrate on which the angle is measured, and the surface smoothness thereof. The contact angle of the substrate for use in the absorbent composite of the invention is determined by putting the substrate on a flat surface of a film or a sheet and applying a drop of distilled water thereonto. That is, the contact angle is to the drop of distilled water, and it may be determined by the use of a device described hereinunder.

The hydrophilic substrate may be prepared by hydrophilicating the surface of a hydrophobic substrate. The hydrophilication treatment may be attained, for example, through modification with an anionic, cationic or nonionic surfactant. The modification may be attained, for example, by directly spraying or applying the surfactant onto a substrate; or by applying it to the substrate in any stage of during or after the preparation of fibers or nonwoven fabrics for the substrate; or adding it to a polymer composition before it is spun into fibers.

On the other hand, polyester-type, polyethylene-type, polypropylene-type, polystyrene-type, polyamide-type, polyvinyl alcohol-type, polyvinyl chloride-type, polyvinylidene chloride-type, polyacrylonitrile-type, polyurea-type, polyurethane-type, polyfluoroethylene-type and polyvinyl cyanide-type hydrophobic substrates are expected to have an effect of dispersing water on their surfaces, and therefore they are usable herein. Hydrophilic fibers and hydrophobic fibers may be combined. For ensuring suitable liquid paths in the absorbent polymer having swollen through absorption, it is desirable to use a substrate having a suitable degree of rigidity. In consideration of environmental problems, preferably used are biodegradable synthetic fibers such as polylactate fibers, aliphatic polyesters.

One or more types of fibers may be used for constituting the fibrous substrate. So far as the absorbent polymer may be bound thereto, one fiber for the substrate may be formed of two or more different types of materials. Regarding their profile, the fibers may be hollow fibers or side-by-side fibers for increasing the liquid dispersiveness and the water conductivity thereof. Regarding the fiber formed of two or more materials, referred to is “a thermoplastic fiber that comprises a core fiber of one material and a thermoplastic sheath of a different material that envelops the core fiber”. Examples of “fibrous sheath/core fiber” suitable to the substrate for the absorbent composite of the invention are those composed of two different types of materials, such as polyethylene/polypropylene, polyethyl acetate/polypropylene, polyethylene/polyester, polypropylene/polyester, polyester copolymer/polyester. Of those, preferred is “a fiber composed of a rigid core of polypropylene or polyester, and a soft sheath of polyester copolymer or polyethylene”, as it has a suitable degree of rigidity and feels soft.

Since the fibrous substrate must have a suitable degree of rigidity and must feel soft, the lowermost limit of the diameter of the fibers that constitute the fibrous substrate is preferably 0.1 dtex, more preferably 1 dtex; and the uppermost limit thereof is preferably 500 dtex, more preferably 100 dtex, even more preferably 50 dtex, most preferably 30 dtex. When the fiber diameter is not larger than the above uppermost limit, then the fibrous substrate may readily envelop and adhere to the absorbent polymer since the fibers may have a suitable degree of rigidity. In addition, the fibers of the type may be readily shaped under compression and are favorably formed into sheet-like and thin substrates where the fibers are entangled. In particular, when the composite is used for sanitary protections and the like, then the fibers do not feel rough and to not irritate skins and have a good feel. On the other hand, when the fiber diameter is not lower than the above lowermost limit, then the fibers are preferable in point of the above-mentioned water conductivity and the diffusiveness, and are effective for preventing lump formation.

The lowermost limit of the fiber length of the fibers is preferably 50 μm, since the fibers of the type are readily shaped and consolidated, more preferably 100 μm, even more preferably 500 μm; and the uppermost limit thereof is preferably 100,000 μm, more preferably 50,000 μm, even more preferably 30,000 μm. When the absorbent polymer is a bound particulate polymer, then the mean particle size of the bound particles/fiber length is preferably from 2/1 to 1/1,000, more preferably from 1/1 to 1/500, even more preferably from 1/2 to 1/100. The fiber length has some influence on the amount of the absorbent polymer bound to one fiber, and when the fibers are long, then the binding between the fibers and the absorbent polymer is strong. In particular, when the fibrous substrate is formed as a sheet, then the fibers for it are preferably long as they may be readily entangled. On the other hand, when the used substrate is desired to be recycled, then the fibers are preferably short as they are readily opened.

Regarding their shape, the fibers may be linear or may be non-linear such as waved, looped, coiled, branched or starlike fibers that may be readily entangled.

The lowermost limit of the unit weight of the fibrous substrate is preferably 5 g/m², from the viewpoint of liquid dispersibility therein, more preferably 10 g/m², even more preferably 20 g/m²; and the uppermost limit thereof is preferably 500 g/m², more preferably 200 g/m², even more preferably 150 g/m². The lowermost limit of the specific volume of the fibrous substrate is preferably 3 ml/g because of excellent fixability of absorbent polymer therein, more preferably 70 ml/g, even more preferably 100 ml/g; and the uppermost limit thereof is preferably 500 g/ml, more preferably 400 ml/g, even more preferably 300 ml/g. The specific volume means the volume (ml) per gram of the substrate. The specific volume is determined by dividing the volume (ml) of the fibrous substrate by the weight thereof. The volume (ml) is determined as follows: A square fibrous substrate sample is analyzed with a digital photomicroscope, and the thickness (cm) of the fibrous substrate in the side direction thereof is measured, and the thus-measured thickness of the fibrous substrate is multiplied by the area thereof.

The fibrous substrate is preferably one having a suitable surface condition, such as paper, fluff pulp, cloth, nonwoven fabric. Fluff pulp is pulp in which the fibers are independently feather-wise entangled, and is characterized in that it is bulky and soft and its absorption is good. Typically, for example, it includes opened products such as NB-416 and FR-416 by Weyerhaeuser. Nonwoven fabric produced by various known methods such an air-laid method, a wet-laid method, a water-jet entraining method, a staple-length fiber curd-bonding method, and a solution spinning method, may be used herein. Nonwoven fabric is generally grouped into air-through, embossed and flat nonwoven fabric having a larger specific volume in that order. Of those, especially preferred are fibrous substrates of fluff pulp and air-through nonwoven fabric such as air-through nonwoven fabric.

In general, fibers tend to be entangled into a mass, but the fibers for the substrate for the absorbent composite of the invention are preferably microscopically uniformly dispersed. The apparent fiber mass diameter is preferably at most 20 mm, more preferably at most 10 mm, even more preferably at most 5 mm. Most preferably, the individual fibers are independent of each other.

In general, the fiber mass can be readily pulverized by opening it, “Opening” is meant to include both concepts of splitting and fibrillation. Splitting includes a technique of splitting sheets of nylon or the like into strips or fibers. Fibrillation includes a technique of beating raw paper-like cellulose into pulp.

Concretely, herein usable are opening devices such as cotton-spinning type, worsted-spinning type, woolen-spinning type, hemp-spinning type, silk-spinning type or rotary blade-type grinders, hammer-type grinders, and pulp beaters. Also employable is a flock-working method where fibers are charged and are uniformly dispersed one by one by utilizing the electrostatic repulsion between the fibers (see Handbook of Fibers (working edition), edited by the Fiber Society of Japan, Maruzen, 1969, p. 18). For the raising machine, herein usable are Santo Engineering's Wet Sander; Naigai Special Engineering's Beaver Emery; Mitsubishi Heavy Industries' MC Roll; Maruichi Giken's Needle Pricker (see JP-A-5-186960, JP-A-2001-254261, JP-A-2001-254262).

### (Raising Treatment)

The substrate to constitute the absorbent composite of the invention is preferably a surface-raised fibrous substrate. The raising treatment is preferably such that the bulk density of the raised fibrous substrate may be from 1/2 to 2/3 of the unprocessed one. The raising treatment means that the surface of the substrate is made fluffy by drawing the constitutive fibers onto the substrate surface or by planting the fibers in the substrate surface. In general, the substrate may be heated, ground (by Emery raising, puffing) or brushed, whereby the fibers may be drawn onto the surface or the fibers may be planted in the surface. For drawing the fibers of the fibrous substrate, employable is a method of grinding the substrate with an abrasive such as sand paper (Emery paper); a method of buffing treatment; a method of brushing with various brushes of metal, plastic such as polyvinyl chloride or pig hair, or with cloth needles (for suede raising) or needle-punching needles; a water-jet method; a method comprising adhering a thermoplastic resin film in melt to the substrate surface and then peeling it; and a modified application method of tufted carpet production, which comprises forming a continuous loop with twisted yarn on the substrate surface, and then cutting the loop by shearing. Fiber plantation may be attained by electric planting. Of those, thermal raising or Emery raising (for puffing) is preferred for the raising treatment for the substrate to constitute the absorbent composite of the invention, as it is inexpensive and is capable of attaining a suitable bulk density. More preferred is thermal raising as causing little danger to fibers. The raising treatment may be carried out once or more, and one or more types of methods may be repeated twice or more.

The raising treatment is generally effected for improving the outward appearance, the touch and the glossiness of the substrate (see Handbook of Fibers (working edition), p. 975, edited by the Fiber Society of Japan, Maruzen, 1969). When the substrate to constitute the absorbent composite of the invention is raised, then its absorbing speed generally increases. This may be because the distance between the absorbent polymer particles bound to the fibrous substrate could be kept appropriate by the raising treatment.

A preferred condition of thermal raising of nonwoven fabric, as one example, is described. The heating temperature may be around the softening point of the base fibers of the nonwoven fabric, practically from 70 to 160° C. The heating time depends on the heating temperature, and may be generally from a few seconds to 180 seconds. More preferably, a substrate is heated at 80 to 140° C. for 20 to 60 seconds. The heating method is not specifically defined, so far as the substrate could be raised by it. For example, a substrate is led through a heating furnace, or hot air is applied to a substrate, or a substrate is irradiated with an IR lamp, or a substrate is kept together with a high-temperature vapor such as steam.

In Emery raising (for puffing), the grain size of the abrasive is preferably from #60 to #1000, more preferably from #100 to #500.

For the raising machine, usable are Santo Engineering's Wet Sander, Naigai Special Engineering's Beaver Emery, Mitsubishi Heavy Industries' MC Roll (see JP-A-5-186960, JP-A-2001-254261, JP-A-2001-254262).

For further increasing the raising effect, proposed are a method comprising applying a polyorganosilicone compound to a composite sheet composed of a fibrous sheet and a polymer elastomer, before or during buffing the sheet (see JP-B-55-32828); a method comprising dissolving or swelling a part of the elastic polymer of a sheet composed of a fibrous material and an elastic polymer, then again solidifying it to tighten the roots of the fibers, thereby raising the sheet (see JP-A-53-31887); a method comprising raising a sheet composed of ultra-fine sized fibers and a high-molecular polymer with wax or paraffin adhered to the sheet (see JP-B-47-44601); a method comprising buffing or raising the surface of nonwoven fabric composed of entangled mixed fibers of an elastic polymer and a nonelastic polymer (see JP-B-1-41742, JP-B-3-79478).

We, the present inventors have found that, when a raised substrate is used, then the gel dropout percentage of the absorbent composite is reduced after absorption and the absorbing speed thereof increases. Further surprisingly, the raising effect does not lower even when the raised substrate is formed into a composite through droplet polymerization thereon and thereafter the resulting composite is shaped under pressure or consolidated.

Though not clear, the effect-expressing mechanism may be owing to the synergistic effect of the specificity to the conformation of the fibers in the substrate surface and the anisotropy in morphology formation in polymerization. The specificity to the conformation of the fibers in the substrate surface is meant to indicate that when a fibrous substrate is raised, then the fibers extends in the direction vertical to the surface of the substrate and the component of the vertically-aligned fibers increases and, in addition, the specific volume of the fibers increases, whereby the substrate surface becomes sparse. On the other hand, the anisotropy in morphology formation in polymerization is described below, differently for the bound particulate absorbent polymer and the web-like absorbent polymer.

The bound particulate absorbent polymer of the invention is generally such that the polymer particles deposited in the vertical direction to the substrate are aggregated (FIG. 34(a)). This is because, when some droplets under polymerization already exist, adhering to the substrate, then droplets under polymerization next dropping onto the substrate tend to adhere to the already-existing droplets rather than to the substrate. In general, when the elasticity and the shape of the substrate may promote bouncing or rolling of droplets, then the droplets dropping onto the substrate may bound with high probability; but on the contrary, when the substrate is sticky, then the probability of fixation of the droplets on the substrate may be high. Since the droplets to give the bound particulate absorbent polymer are sticky and elastic, it may be considered that the droplets would aggregate in the accumulation direction, or that is in the vertical direction. When a raised substrate is used, then the bound particulate absorbent polymer and the web-like absorbent polymer may be uniformly dispersed in the substrate, and even in the depth of the substrate, the absorbent polymer may be bound to the substrate thereby resulting in that the binding points of the absorbent polymer may increase (FIG. 34(b)). On the other hand, the web-like absorbent polymer of the invention expands horizontally to the surface of the substrate, and when its unit weight is larger, then it may cover more broadly the substrate surface (FIG. 35(a)). When a raised substrate is used, then the web structure may be formed along the raised fibers (FIG. 35(b)).

In both cases of the absorbent polymers of those types, the absorbent polymer is uniformly dispersed in the substrate and therefore the distance between the substrate-substrate, between the substrate-absorbent polymer, and between the absorbent polymer-absorbent polymer could readily undergo a capillary phenomenon. Naturally, in addition, since the distance suitable to a capillary phenomenon is extremely small, the compression treatment and the consolidation treatment would not lower the absorbing speed of the processed substrate. Accordingly, even in the depth of the substrate, the absorbent polymer is kept bound to the substrate and the binding points of the absorbent polymer increases, and, in addition, since the binding sites between the substrate and the absorbent polymer can be relatively freely as a result of the raising treatment, the impact stress and the stress after absorption may be relaxed, therefore resulting in that the polymer dropout percentage may be reduced and the gel dropout percentage after absorption may also be reduced.

These effects are derived from the anisotropy in morphology formation in polymerization in the invention. No one has heretofore expected those excellent effects by raising. In particular, no one has heretofore expected that the raising effects wound not be reduced even when the raised substrate is subjected to droplet polymerization and is formed into a composite and even when the composite is shaped under pressure or consolidated to reduce its specific volume. In fact, when a powdery absorbent polymer is sprayed onto a substrate and mixed, and then pressed or consolidated and when the resulting composite is raised, then the method could not produce a remarkable advantage in the absorbing speed, the gel dropout percentage after absorption and the gel dropout percentage of the resulting composite.

There are known some documents describing a raised absorbent composite (JP-A-2004-91996, JP-A-2004-149970). Concretely, there is a description saying that “an aqueous monomer solution consisting essentially of acrylic acid and/or its salt is sprayed onto a thermally-raised fibrous substrate to thereby obtain a fibrous substrate having the aqueous monomer solution held thereon as fine particles thereof, and then the monomer on the fibrous substrate is polymerized”. In this, a raised substrate is used, but the polymerization method differs from the method of the invention. In addition, this explicitly says that “independently separated, nearly spherical fine particles are held like beads”. This differs from the “absorbent composite that comprises, bound to a substrate, an aggregated absorbent polymer of two or more, nearly spherical absorbent polymer particles aggregated together and a web-like absorbent polymer” of the invention in point of the structure of the composite.

The unit weight of the absorbent polymer in the absorbent composite of the invention is preferably from 10 to 1000 g/m² in terms of the dry weight thereof in measurement of the water-holding ability of the composite to be mentioned hereinunder.

### III. Method for Producing Absorbent Composite

### (Binding Between Absorbent Polymer and Substrate)

Not specifically defined, the method for producing the absorbent composite of the invention may be any one capable of producing the absorbent composite that satisfies the condition described in the claims. The absorbent composite of the invention may be produced efficiently when the absorbent polymer produced through polymerization according to the above-mentioned preferred method is used. In particular, it is desirable that the absorbent composite is produced by contacting droplets that contain a polymerizable monomer and a redox-type polymerization initiator with a substrate. The conversion of the polymerizable monomer in contact of the droplets with a substrate must be low so that the substrate could be buried in or could adhere to the absorbent polymer, but must be high so that the droplets are sticky to the substrate. Accordingly, the lowermost limit of the conversion is preferably 20%, more preferably 30%, even more preferably 40%, most preferably 50%; and the uppermost limit thereof is preferably 95%, more preferably 80%, even more preferably 60%, most preferably 55%. The conversion of the polymerizable monomer in contact with the substrate may be controlled by changing the dropping distance of the droplets.

Not adhering to any theory, the reason why the bound particulate absorbent polymer could be produced according to the production method for the absorbent composite of the invention would be as follows: In the production method for the absorbent composite of the invention, droplets under polymerization are contacted with a substrate. In this, since the substrate has fine projections and recesses, the adhesive droplets are aggregated and bound into particles on the substrate. In addition, while the droplets are aggregated, they engulf the substrate and the substrate is thereby buried in the particles or adheres to the surfaces of the particles, resulting in that the substrate is bound to the absorbent polymer to form the absorbent composite of the invention. When the bound particulate absorbent polymer has a low degree of polymerization (that is, when it is soft) while the droplets under polymerization are contacted with the substrate, then the substrate may be readily buried in the absorbent polymer; but when the degree of polymerization of the polymer is high (that is, when the polymer is hard), then the substrate may readily adhere to the absorbent polymer.

Especially preferably, the above droplet polymerization includes both the step where the droplets are kept in contact with the substrate while the conversion of the polymerizable monomer is low and the step where the droplets are kept in contact with the substrate while the conversion of the polymerizable monomer is high. Preferably, the conversion in the former step is at most 40%, more preferably at most 30%, even more preferably at most 25%, most preferably at most 20%. The conversion in the latter step is preferably at least 40%, more preferably at least 45%, even more preferably at least 50%. In the step where the droplets are kept in contact with the substrate while the conversion of the polymerizable monomer is low, the web-like absorbent polymer may be formed; and in the step where the droplets are kept in contact with the substrate while the conversion of the polymerizable monomer is high, the bound particulate absorbent polymer of at least two nearly-spherical absorbent polymer bound to each other may be formed. This may be attained, for example, by supplying the substrate to polymerization columns that differ in point of the conversion of the polymerizable monomer therein. Concretely, for this, the polymerization apparatus may be so designed that plural polymerization columns that differ in point of the height of the nozzle for supply of droplets (the dropping distance of droplets) thereinto are provided with plural substrate-supply columns. Concretely preferred are the following two embodiments of the production method for the absorbent composite that comprises the two steps.

One embodiment of the production method is a successive method comprising contacting the droplets with substrates while the conversion of the polymerizable monomer is low and contacting the droplets with the substrate while the conversion of the polymerizable monomer is high. Comprising these two steps, any of these steps in the method may be carried out first. Each step may be carried out plural times. The distance between the first step and the second step may vary depending on the ambient temperature, but in general, it is preferably from 0.1 seconds to 1200 seconds, more preferably from 0.5 seconds to 600 seconds, even more preferably from 1 second to 300 seconds. Mainly depending on the time of each step, the ratio by weight of the web-like absorbent polymer to the bound particulate absorbent polymer maybe controlled.

The second embodiment of the production method is a simultaneous method where the step of contacting the droplets with a substrate while the conversion of the polymerizable monomer is low and the step of contacting the droplets with the substrate while the conversion of the polymerizable monomer is high are carried out simultaneously. When the two steps are carried out simultaneously, then the production step of the method is high. For forming the droplets, generally used are nozzles. The nozzles for the respective steps may be so disposed that the spaces where the droplets drop down may overlap with each other, or may not overlap with each other. Preferred is the method where the nozzles are so disposed that the spaces may overlap with each other. The distance between the nozzles for the respective steps may be generally from 0.5 cm to 100 cm in terms of the distance between the center lines of the nozzles, preferably from 1 cm to 50 cm, more preferably from 2 cm to 30 cm.

For forming both the partly-buried substrate and the surface-adhering substrate, it is desirable that the difference in the conversion between the polymerizable monomers at the time of contact with the substrate from 10 to 80%, more preferably from 10 to 70%, most preferably from 10 to 60%.

### (Drying Treatment)

The absorbent composite obtained according to the above-mentioned method is generally dried. In case where a remaining monomer treatment to be mentioned below is carried out, the drying treatment is preferably carried out after the remaining monomer treatment. In case where the absorbent composite is wetted with a medium such as water or a solvent or with water vapor such as steam in any other additional step, the drying treatment is preferably carried out after the additional treatment. In the drying treatment, in general, the composite is dried so that the water content thereof could be at most 10% by weight. From the viewpoint of the quality of the product (in point of the difficulty in causing the degradation, such as the increase in impurities owing to cutting of the polymer chains of the absorbent polymer, and the increase in water-soluble ingredients) and the drying efficiency (time to be taken for the drying), the drying temperature is preferably set within a range of from 100 to 150° C.

### (Other Additional Steps)

The production method for the absorbent composite of the invention may further include remaining monomer treatment, surface-crosslinking treatment, treatment for imparting any other function to the product, shaping, heat-seal treatment, consolidation treatment, thin film formation treatment, recycling treatment.

### (Remaining Monomer Treatment)

The production method for the absorbent composite of the invention preferably includes a treatment for reducing the remaining monomer. In case where the composite is subjected to shaping treatment or additive treatment that is mentioned hereinunder, the treatment for reducing the remaining monomer from it is generally effected before that treatment. The method of reducing the remaining monomer includes three embodiments of 1) further promoting the polymerization of the remaining monomer, 2) converting the remaining monomer into any other derivative, and 3) removing the remaining monomer.

The remaining monomer in the absorbent polymer may be quantified generally by extracting out the remaining monomer with water from the absorbent polymer followed by determining its concentration. The concentration determination may be attained through high-performance liquid chromatography (LC) or gas chromatography (GC). The suitable amount of the remaining monomer not causing odor and corrosion and not having toxicity may vary depending on the use of the absorbent composite, but in general, it is preferably at most 10000 ppm for non-sanitary materials, more preferably at most 5000 ppm, even more preferably at most 2000 ppm. For sanitary materials such as diapers, it is preferably at most 2000 ppm, more preferably at most 1000 ppm, even more preferably at most 500 ppm, most preferably at most 300 ppm.

The method 1) for promoting the polymerization of the remaining monomer includes, for example, heating the system, contacting it with a polymerization promoter, or irradiating it with energy rays such as UV rays, electron rays or radiation rays. The treatment of reducing the remaining monomer by heating the system comprises, in general, heating the absorbent composite at 100 to 250° C. to thereby further polymerize the remaining monomer.

In the method of using a polymerization promoter, in general, the absorbent composite is first contacted with a substance capable of promoting the polymerization of the remaining monomer and then it is heated. For example, when a redox-type polymerization initiator is used in polymerization and when the oxidizing agent has remained in the system, then a reducing agent may be contacted with the system; but on the contrary, when a reducing agent has remained, then an oxidizing agent may be contacted with it. The reducing agent to be used as the polymerization promoter may include the same as those mentioned hereinabove for the reducing agent for the above-mentioned redox-type polymerization initiator. Concretely, it includes sodium sulfite, sodium hydrogensulfite, L-ascorbic acid. The oxidizing agent to be used as the polymerization promoter may include the same as those mentioned hereinabove for the oxidizing agent for the above-mentioned redox-type polymerization initiator. Concretely, it includes peroxides such as hydrogen peroxide, t-butylhydroperoxide, cumenehydroperoxide; persulfates such as ammonium persulfate, potassium persulfate; ceric salts; permanganates chlorites; hypochlorites; and other inorganic salts. The polymerization promoter is contacted with the absorbent composite generally as an aqueous solution thereof having a concentration of from 0.5 to 5% by weight. The amount of the polymerization promoter may be preferably from 0.1 to 2% by weight of the dry absorbent polymer. The polymerization promoter may be contacted with the absorbent polymer according to a method of spraying the polymerization promoter onto the absorbent polymer with a spray, or a method of dipping the absorbent polymer in a solution of the polymerization promoter. Regarding the heating condition in the polymerization promoter treatment with, for example, L-ascorbic acid (reducing agent), in general, the system may be heated at 100 to 150° C. for 10 to 30 minutes. The heating lowers the water content of the absorbent composite. When the water content is desired to be lowered more, then the composite may be dried.

In the method of irradiating the absorbent composite with energy rays such as UV rays, electron rays or radiation rays, any irradiation device may be used with no specific limitation thereto. For example, herein employable is a device where an absorbent composite is kept statically and is irradiated with energy rays for a predetermined period of time. Another device is also employable where an absorbent composite is continuously conveyed on a belt conveyor and is irradiated with energy rays. The irradiation condition including the irradiation intensity and the irradiation time may be suitably determined depending on the type of the substrate and the amount of the remaining monomer. Not significantly detracting from the excellent advantages of the absorbent composite of the invention, the irradiation condition is not specifically defined.

As the UV irradiation device, employable is an ordinary UV lamp. The intensity of the UV lamp is preferably from 10 to 200 W/cm, more preferably from 30 to 120 W/cm. Preferably, the distance between the lamp and the composite is from 2 to 30 cm; and preferably, the irradiation time is from 0.1 seconds to 30 minutes. The temperature at irradiation may be room temperature. The irradiation atmosphere may be an inert atmosphere such as nitrogen, argon or helium, or may be air. The pressure at irradiation may be increased pressure or normal pressure or reduced pressure (including vacuum). The water content of the absorbent composite during irradiation may be preferably from 0.01 to 40 parts by weight relative to 1 part by weight of the absorbent polymer, more preferably from 0.1 to 1.0 part by weight, since the remaining monomer in the composite is movable and since the UV transmittance of the absorbent polymer therein is good under the condition. In order that the water content of the absorbent polymer may fall within the preferred range for UV irradiation, the absorbent composite may be previously subjected to the above-mentioned drying treatment.

The radiation rays include high-energy radiation rays of accelerated electrons or gamma rays. In general, the irradiation dose may be generally from 0.01 to 100 Mrad, preferably from 0.1 to 50 Mrad, since the composite thus processed may have a suitable absorption and a suitable absorbing speed. The temperature at irradiation may be room temperature. The irradiation atmosphere may be an inert atmosphere such as nitrogen, argon or helium, but is preferably air since the processed composite may have a suitable absorption and a suitable absorbing speed and since the remaining monomer amount therein may be readily reduced to at most 500 ppm. The pressure at irradiation may be increased pressure or normal pressure or reduced pressure (including vacuum). The water content of the absorbent composite during irradiation may be preferably from 0.01 to 40 parts by weight relative to 1 part by weight of the absorbent polymer, more preferably from 0.1 to 1.0 part by weight, since the remaining monomer in the composite is movable and since the UV transmittance of the absorbent polymer therein is good under the condition. In order that the water content of the absorbent polymer may fall within the preferred range for radiation rays, the absorbent composite may be previously subjected to the above-mentioned drying treatment.

The method 2) of converting the remaining monomer into any other derivative comprises, for example, adding a reducing agent, such as ammonia substituents, e.g., amine, ammonia, or hydrogensulfites, sulfites or pyrosulfites, to the absorbent polymer after polymerization, in an amount of from 0.001 to 5.0 parts by weight of the polymer.

The method 3) of removing the remaining monomer comprises, for example, extracting and evaporating away the remaining monomer with an organic solvent. The method of extracting it with an organic solvent may be attained generally by dipping the absorbent composite in a water-containing organic solvent. The water-containing organic solvent may be a combination of a water-soluble organic solvent such as methanol, ethanol or acetone and water. In general, when the water content of the water-containing organic solvent is higher, then the remaining monomer-removing ability thereof may be higher. However, if it is too high, the energy consumption in the subsequent drying step may increase. Therefore, the water content of the water-containing organic solvent is preferably from 10 to 99% by weight, more preferably from 30 to 60% by weight. The time for which the absorbent composite is dipped in such a water-containing organic solvent may be generally from 5 to 30 seconds. When the absorbent composite is dipped in a water-containing organic solvent, it is desirable to employ a method of promoting the remaining monomer extraction from it, for example, by shaking the absorbent composite. After the dipping treatment, in general, the composite is dried with a drier.

For evaporating away the remaining monomer, employable is a method of processing the absorbent composite with an overheated steam-containing gas. For example, saturated steam at 110° C. is further heated up to 120 to 150° C., and contacted with the absorbent composite, whereby the remaining monomer in the absorbent polymer may be reduced. According to this method, the remaining monomer may also be vaporized and removed from the absorbent polymer while water is evaporated away as steam from the absorbent polymer. According to the method, the removal of the remaining monomer and the drying of the product can be attained simultaneously.

### (Surface-Crosslinking Treatment)

In the production method for the absorbent composite of the invention, the composite is preferably surface-crosslinked with a crosslinking agent. In general, when the surface of the absorbent polymer is crosslinked, then the absorbent polymer may keep its surface shape while it has absorbed liquid and has swollen, and therefore the absorbent polymer may be prevented from adhering to each other to be deformed and may hardly cause a phenomenon of fluid path blocking in the absorbent composite (this may be referred to as “gel blocking phenomenon”), and the absorbing speed of the composite may be thereby increased. The absorbent polymer may be crosslinked generally by contacting a crosslinking agent and water with the surfaces of powdery absorbent polymer particles followed by heating them. According to the treatment, a crosslinked structure may be selectively formed on the surface, and the swelling failure in absorption may be reduced, and therefore the absorbent polymer may well keep its shape.

For the surface-crosslinking treatment, preferred is a simple method of contacting a crosslinking agent solution with an absorbent composite followed by heating it to attain the intended crosslinking. The crosslinking agent may be the same as that used in the above-mentioned polymerization. The amount of the crosslinking agent to be sued may be generally from 0.1 to 1% by weight of the absorbent composite, preferably from 0.2 to 0.5% by weight. In order that the surface of the absorbent composite can be uniformly treated, it is desirable that the crosslinking agent is used as a solution in water, ethanol or methanol having a concentration of from 0.1 to 1% by weight, more preferably from 0.2 to 0.5% by weight. Concretely, herein employable is a method of spraying a crosslinking solution with a spray; or a method of applying it with a roll brush. As the case may be, an excessive amount of a crosslinking agent solution may be contacted with an absorbent composite and the excessive solution may be removed by lightly compressing the absorbent composite to such a degree that the composite is not broken or by applying an air jet to the composite. In general, the crosslinking agent solution is contacted with an absorbent composite at room temperature. The crosslinking reaction condition may be suitably selected depending on the crosslinking agent used. In general, the agent is reacted at a temperature not lower than 100° C. for at least 10 minutes.

### (Additive Treatment)

The absorbent composite of the invention may be subjected to additive treatment for making it have various functions given thereto. The functions to be given include improvement of the performance and the quality of the absorbent polymer itself, and the improvement of the quality of the absorbent article formed with the absorbent composite. A chemical agent capable of improving the performance and the quality of the absorbent polymer itself includes an oxidizing agent, a reducing agent, a foaming agent, a foaming promoter. A chemical agent capable of improving the quality of the absorbent article includes a stabilizer that acts to stabilize the absorbent polymer in contact with body discharges; an antimicrobial agent, an odor remover, a deodorizer and an aroma that act to relieve the odor and the corruption smell of body discharges; a pH-controlling agent that acts to increase the safety of the absorbent composite in contact with skins; a chemical agent and a neutralizing agent that act to keep skins weakly acidic.

The treatment with these additives may be suitably attained in various steps constituting the process of producing the absorbent composite, depending on the object and the effect and the mechanism of the composite. The foaming agent is preferably used in the step of producing the absorbent polymer. Concretely, it is preferably added to the system before or during polymerization. The chemical agents for improving the quality of the absorbent article are preferably added in the process of from “the step of producing the absorbent composite” to “the step of producing the absorbent article”. These additives may also be used in any other ingredients than the absorbent composite constituting the absorbent article.

In case where the reducing agent or the oxidizing agent used in producing the absorbent composite has remained in the composite, an oxidizing agent or a reducing agent may be added to the composite and it may act to decompose the remaining agent. The oxidizing agent and the reducing agent may include the same as those mentioned hereinabove for the above-mentioned oxidizing agent and the above-mentioned reducing agent. When the reducing agent or the oxidizing agent used in producing the absorbent composite has remained in the composite, then it may be decomposed by heat or light and may cut the main chain of the absorbent polymer to increase water-soluble ingredients, or may produce decomposed products to cause discoloration or odor generation.

The foaming agent and the foaming promoter may be effective for making the absorbent polymer porous to thereby increase the surface area thereof, and for improving the absorbing performance of the polymer. The foaming agent and the foaming promoter include pyrolytic gas-generating compounds such as sodium bicarbonate, nitroso compounds, azo compounds, sulfonyl hydrazide (see Additive Chemicals to Rubbers and Plastics, by Rubber Digest, 1989, pp. 259-267).

The stabilizer is a substance for preventing the absorbent polymer from being decomposed and degraded by discharges (urine, feces), body fluids (blood, menstrual discharge, secretions). Concretely, herein employable is a method of adding such a stabilizer to the absorbent polymer. The stabilizer includes oxygen-containing reducing inorganic salts and/or organic antioxidants (see JP-A-63-118375), oxidizing agents (see JP-A-63-153060), antioxidants (see JP-A-63-127754), sulfur-containing reducing agents (see JP-A-63-272349), metal chelating agents (see JP-A-63-146964), radical chain reaction inhibitors (see JP-A-63-15266), phosphinic acid group and phosphonic acid group-containing amine compounds and their salts (see JP-A-1-275661), polyvalent metal oxides (see JP-A-64-29257), water-soluble chain transfer agents (to be added during polymerization) (see JP-A-2-255804, 3-179008). In addition, also usable are potassium oxalate titanate, tannic acid, titanium oxide, phosphinic acid amine (or its salt), phosphonic acid amine (or its salt), metal chelates (JP-A-6-306202, JP-A-7-53884, JP-A-7-62252, JP-A-7-113048, JP-A-7-145326, JP-A-7-145263, JP-A-7-228788, JP-A-7-228790). Of those, the stabilizers for human urine, human blood and menstrual discharge may be referred to as “human urine stabilizer”, “human blood stabilizer”, and “menstrual discharge stabilizer”, respectively.

The antimicrobial agent is a substance to be used for preventing corruption by the absorbed liquid. The antimicrobial agent may be grouped into nitrogen compounds, substituted phenols, metal compounds, rare earth salts of surfactants, silver-based inorganic powders. The nitrogen compounds may be grouped into cyclic nitrogen compounds and acyclic nitrogen compounds. For the antimicrobial agent, referred to are New Development of Microbicidal and Antimicrobial Technology, pp. 17-80 (by Toray Research Center, 1944); Inspection and Evaluation Method and Product Planning of Antibacterial and Antifungal Agents, pp. 128-344 (by NTS, 1997); Japanese Patent 2760814; JP-A-39-179114, JP-A-56-31425, JP-A-57-25813, JP-A-59-189854, JP-A-59-105448, JP-A-60-158861, JP-A-61-181532, JP-A-63-135501, JP-A-63-139556, JP-A-63-156540, JP-A-64-5546, JP-A-64-5547, JP-A-1-153748, JP-A-1-221242, JP-A-2-253847, JP-A-3-59075, JP-A-3-103254, JP-A-3-221141, JP-A-4-11948, JP-A-4-92664, JP-A-4-138165, JP-A-4-266947, JP-A-5-9344, JP-A-5-68694, JP-A-5-161671, JP-A-5-179053, JP-A-5-269164, JP-A-7-165981.

The cyclic nitrogen compounds are typically quaternary nitrogen compounds. The cyclic quaternary nitrogen compounds include alkylpyridinium salts and pyrithione salts. The alkylpyridinium salts include dodecylpyridinium chloride, tetradecylpyridinium chloride, cetylpyridinium chloride (CPC), tetradecyl-4-ethylpyridinium chloride, tetradecyl-4-methylpyridinium chloride; and the pyrithione salts include pyrithione zinc.

The acyclic nitrogen compounds may be grouped into quaternary nitrogen compounds and polynitrogen compounds. The quaternary nitrogen compounds include methylbenzethonium chloride, benzalkonium chloride, dodecyltrimethylammonium bromide, tetradecyltrimethylammonium bromide, hexadecyltrimethylammonium bromide. The acyclic quaternary nitrogen compounds include benzalkonium chloride.

The acyclic polynitrogen compounds include guanidine derivatives, polyguanides. The polyguanides include bis-guanides (see U.S. Pat. Nos. 2,648,924, 2,990,425, 2,830,006, 2,863,019). The bis-guanides are imidopolycarbonimidic acid-diamides derived from carbonic acid. Of those, preferred is 1,6-bis(4-chlorophenyl)diguanidohexane. 1,6-Bis(4-chlorophenyl)diguanidohexane includes chlorohexidine and its water-soluble salts. 1,6-Bis(4-chlorophenyl)diguanidohexane is especially preferably chlorohexidine hydrochloride, acetate and gluconate.

The acyclic mononitrogen compounds include carbanilides. The carbanilides are aniline derivative phenylcarbamates, including 3,4,4′-tirchlorocarbanilide (TCC, trichlorocarban) and 3-(trifluoromethyl-4,4′-dichlorocarbanilide (IRGASAN).

The substituted phenols usable for antimicrobial agent include 5-chloro-2-(2,4-dichlorophenoxy)phenol (IRGASAN DP-300). The metal compounds usable for antimicrobial agent include graphite and tin salts. They also include zinc chloride, zinc sulfide, tin chloride. The rare earth salts of surfactants usable for antimicrobial agent include C₁₀ to C₁₈ linear alkylbenzenesulfonates with lanthanum (see EP Laid-Open 10819).

The odor remover, the deodorant and the aroma are effective for preventing or reducing the odor generation by liquid absorption. Concretely, the odor remover and the deodorant include iron complexes, tea extracts and activated charcoal. The aroma includes perfumes (citral, cinnamic aldehyde, heliotropin, camphor, bornyl acetate), pyroligneous acid, paradichlorobenzene, surfactants, higher alcohols, terpene compounds (limonene, pinene, camphor, borneol, eucalyptol, eugenol) (see Technology and Prospects of New Odor Removers and Deodorants (by Toray Research Center, 1994); JP-A-59-105448, JP-A-60-158861, JP-A-61-181532, JP-A-1-153748, JP-A-1-221242, JP-A-1-265956, JP-A-2-41155, JP-A-2-253847, JP-A-3-103254, JP-A-5-269164, JP-A-5-277143).

The pH-controlling agent and the chemical and the neutralizing agent for keeping skins weakly acidic include natural fruit acids (e.g., malic acid, succinic acid, citric acid, tartaric acid, lactic acid), alkali metal salts and alkaline earth metal salts (e.g., phosphates, carbonates).

### (Shaping Treatment)

In the production method for the absorbent composite of the invention, the composite is preferably subjected to shaping treatment. It may be because, in the shaped absorbent composite, the substrate on the surface side of the absorbent polymer and that on the back side thereof may be kept close to each other sufficiently to such a degree that they may be kept in contact with each other via the pores or the voids of the absorbent polymer or to such a degree that they may induce a capillary phenomenon for the liquid to be absorbed. Shaping means that the composite is shaped in accordance with its use. Shaping includes, for example, three-dimensionally shaping the composite into a shape of bowl, vat or saddle; making the composite have projections and recesses, or making it have gathers; embossing the composite to have a predetermined design or pattern. The shaping may be attained in any method capable of shaping the absorbent composite into a desired form. As simple, compression shaping is preferred. The pressure for compression shaping is preferably over the yield point of the substrate so that the absorbent composite thus shaped under the pressure could no more be restored to its original condition. Concretely, the pressure is preferably from 5 to 20 MPa, more preferably from 7 to 15 MPa.

### (Heat-Seal Treatment)

In the production method for the absorbent composite of the invention, when the substrate is thermoplastic, then the composite may be heated at a temperature not lower than the melting point or the softening point of the substrate but lower than the degradation temperature thereof (in general, the temperature range is referred to as a shaping temperature) for heat-sealing it. Concretely, for example, in case where a thermoplastic substrate is disposed on both sides of the absorbent polymer, the composite is heated under pressure at the shaping temperature of the substrate in any desired pattern of dots, lines or lattices, whereby the substrates are partly fused to each other and the resulting composite may have high resistance to rubbing stress. Any additional thermoplastic material may be heat-sealed onto the side of the thermoplastic substrate of the composite that is opposite to the side thereof bound to the absorbent polymer.

### (Consolidation Treatment)

In the production method for the absorbent composite of the invention, the composite produced may be subjected to consolidation treatment. Consolidation treatment comprises pressing the absorbent composite, and is effective for increasing the density of the resulting absorbent composite, increasing the absorbing speed of the composite and enhancing the binding between the absorbent polymer and the substrate. The reason why the absorbing speed increases may be because the contact or close disposition between the absorbent polymer and the substrate or between the substrates may induce a capillary phenomenon. The pressing for compression shaping is attained essentially for shaping. On the other hand, the pressing for consolidation is attained essentially for increasing the density of the composite and improving the performance thereof. In this, one pressing operation may be for both the two (shaping and consolidation).

The device for consolidation may be, for example, a presser such as a plate presser or a roll presser. The pressure for consolidation is preferably from 5 to 20 MPa, more preferably from 7 to 15 MPa. When the pressure is lower than the above uppermost limit, it is favorable since the absorbent polymer may be hardly broken to release its debris, or the absorbent polymer may hardly deviate from the substrate during absorption. During consolidation, the composite may be heated. However, the heating temperature is preferably not so high in order that the substrates may not be bound together to form a network, thereby detracting from the liquid perviousness and the softness of the absorbent composite. Especially preferably, the heating temperature is not higher than the melting point of the substrate. During the consolidation treatment, the composite may be humidified so as to increase the surface hydrophilicity thereof. In general, the humidification may be effected with water vapor.

### (Treatment for Thickness Reduction)

In case where the absorbent composite of the invention is formed into a thin absorbent article, it is desirable that the absorbent composite is thinned. The thickness of the absorbent composite is preferably from 0.2 to 10 mm, more preferably from 0.4 to 2.5 mm, even more preferably from 0.6 to 1.5 mm. The bulk density of the absorbent composite suitable to the range is preferably from 0.20 to 1.10 g/cm³, more preferably from 0.20 to 0.85 g/cm³. Preferably, the bulk density of the absorbent polymer sheet is from 0.20 to 1.10 g/cm³, more preferably from 0.30 to 0.85 g/cm³, even more preferably from 0.40 to 0.85 g/cm³.

For thinning the absorbent composite of the invention, employable is any method not significantly detracting from the excellent properties of the composite. For example, the composite may be pressed.

Pressing the composite may be effected with a presser such as a plate presser or a roll pressure. The pressure for pressing may be selected within a range within which the absorbent polymer is not broken. If the absorbent polymer is broken, then it may peel from the substrate and may drop from the absorbent composite and the absorbent gel may drop from the substrate during swelling, therefore worsening the performance of the absorbent composite.

In general, a fibrous substrate may be such that when it is compressed by pressure but when pressing it is stopped, then the thickness of the substrate may be restored to the original thickness before compression. This property is referred to as a compression restorability. For reducing the compression restorability, heat treatment is effective. For a hydrophilic fibrous substrate, wetting treatment is also effective for reducing the compression restorability thereof.

The heat treatment is preferably effected at a temperature not higher than the melting point of the substrate since network formation owing to the binding of the substrates hardly occurs at that temperature and since the softness and the feel of the treated composite may be good.

For example, the heat treatment may be effected by spraying water onto the absorbent composite or by applying water vapor thereto. The amount of moisture to be applied to the absorbent composite may be suitably selected depending on the content of the absorbent polymer in the composite. The amount of moisture to be applied may be generally at most 500 g/m², preferably at most 300 g/m², more preferably at most 100 g/m². The range is preferred for the following reasons: Within the range, the absorbent polymer is hardly softened (crushed), the binding strength of the bound particles is high, the substrates are hardly bound together to form a network, the softness and the feel of the composite may be good, and the water content increased by moisture application may be readily reduced. In case where moisture is applied to the composite as water vapor, then the water vapor pressure is preferably lower than 10 MPa, more preferably lower than 1 MPa since the moisture applied may not penetrate into the absorbent polymer and since the absorbent polymer hardly swells. The water vapor supply speed may be suitably selected depending on the content of the absorbent polymer in the absorbent composite and on the time for the humidification treatment. The water vapor supply speed may be generally at most 300 kg/hr per m² of the absorbent composite, preferably at most 100 kg/hr, more preferably at most 50 kg/hr. The treatment time may be generally at most 1 hour, preferably at most 30 minutes, more preferably at most 20 minutes.

A typical embodiment of the absorbent composite of the invention is a two-layered structure comprising an absorbent polymer bound onto a substrate. In case where the absorbent composite of the invention is used for an absorbent article, then, in general, any other material is laminated on the side of the absorbent polymer thereof. In case where the structure is subjected to compression shaping, consolidation treatment or thickness-reducing treatment, then it is preferably pressed along with the laminated material in view of the fixability of the laminated material and of the protection of the absorbent polymer. In case where a substrate is not laminated on the absorbent composite, then a masking sheet may be provided on the surface of the absorbent polymer during the compression shaping treatment, consolidation treatment or thickness-reducing treatment for protecting the absorbent polymer, and then it may be removed after the treatment.

### (Recycling Treatment)

The wastes, the remainders and the utilities in the respective steps in the production method for the absorbent composite of the invention are preferably recycled or reused in the recycling step for them from the viewpoint of effective use of natural resources and of reduction in wastes. Concretely, it is desirable that the absorbent polymer is recovered from the materials broken in the production step, the goods returned from the market owing to soiling or outside the standard, and the used goods. Regarding the recovery of the absorbent polymer, for example, there is known a method of shaking sanitary protections to sieve an absorbent polymer powder from it and screening it (see JP-A-2003-39023). The absorbent composite of the invention is preferably a recyclable one. Concretely, for example, it is desirable to use a substrate capable of being readily opened and screened, such as non-shaped fibers. In the absorbent composite described in JP-A-11-93073, nearly spherical absorbent polymer particles are discontinuously fixed on the surface of non-shaped fibers for the purpose of improving the shape retentive stability of the absorbent polymer sheet therein. Rather than the three-dimensional network of non-shaped fibers bonding to each other via absorbent polymer particles as in the above, preferred are unshaped fibers that are entangled by themselves as they are readily opened.

For opening the fibers, employable are any suitable methods using various known opening devices, like those described hereinabove in the section of substrate fibers. Of those, preferred are opening devices provided with a large number of pins or blades. The device and the condition for the opening are preferably such that the absorbent polymer is not broken by mechanical impact applied thereto. The opening may be effected according to a method where cylinders each having needle-like pins are rotated in the same direction and fibers are combed and opened between the cylinders; or a method where cylinders are rotated in opposite directions so that fibers may be made to collide against the housing and are thereby opened by utilizing the impact force by centrifugal force (see JP-A-5-9813, JP-A-6-57542); or a method where a cylinder having needle-like pins and an auxiliary plate having needle-like pins are used and an absorbent composite is mechanically opened while the substrate is prevented from being broken.

Preferably, the opened absorbent composite is sieved using a device under the condition under which the absorbent polymer is not broken. Concretely, for example, using any one or both of known forced stirring operation and forced vibration operation, the opened fibers may be sieved by sucking them under reduced pressure from the opposite side to the mesh. The opening and the sieving may be effected simultaneously.

### (Production Apparatus for Absorbent Composite)

With reference to FIG. 11 and FIG. 12, constitutive examples of typical production devices for the absorbent composite of the invention are described below.

In FIG. 11, 101 is a tower where non-shaped fibers are deposited; 102 is a polymerization tower; 103 is a surface-treatment agent spray; 104 is a drier tower; 105 and 106 are driving rollers; 107 is a belt (mesh belt) conveyor hung over the driving rollers 105 and 106. Using this, an absorbent composite is continuously produced according to the following process.

Fibers S from the fiber deposition tower 101 are deposited on the belt conveyor 107. On this, particles J under polymerization are put from the polymerization tower 102. Using a surface-treatment agent spray 103, a surface-treatment agent is sprayed onto it. This is led to pass through the drier tower 104, in which this is dried. This is pressed between the press rollers 108. This is wound up with the winder 109.

In FIG. 11, the non-shaped fiber deposition tower 101, the polymerization tower 102 and the surface-treatment agent spray 103 each may be divided into two stages. FIG. 12 shows the modification where they are divided in two stages. In FIG. 12, 101A and 101B are non-shaped fiber deposition towers; 102A and 102B are polymerization towers; 103A and 103B are surface-treatment agent sprays. In FIG. 12, the drier tower is not shown.

This is described in more detail. It is desirable that the non-shaped fibers are put into the non-shaped fiber deposition tower 101 so that they may be uniform by pneumatic conveyance. The non-shaped fibers S having dropped down in the non-shaped fiber deposition tower further drop down and are deposited uniformly on the belt conveyor 107. In this, an aspirator 201 may be provided below the non-shaped fiber deposition tower 101 for suction from the back side of the belt conveyor so as to promote the deposition of the fibers. Two or more different types of non-shaped fibers may be mixed and supplied to the device. Two or more non-shaped fiber deposition towers may be disposed in series so that two or more different types of non-shaped fibers may be deposited. The non-shaped fibers thus deposited on the belt conveyor 107 are conveyed to the polymerization tower 102. In the polymerization tower 102, droplets of a polymerizable monomer drop down onto the non-shaped fibers while they are polymerized.

In the above-mentioned typical production method, the production condition may be suitably determined and various forms of absorbent composites may be produced. Three typical patterns of the absorbent composites are described below.

The first pattern is an absorbent composite having two or more substrate layers. This may be produced by the use of a production device having two-staged or more multi-staged non-shaped fiber deposition towers (see FIG. 12), in which different types of fibers are put in the respective non-shaped fiber deposition towers (for example, the first non-shaped fiber deposition tower 101A and the second non-shaped fiber deposition tower 102B in FIG. 12). In this, the fiber supply amount from each non-shaped fiber deposition tower (for example, the first non-shaped fiber deposition tower 101A and the second non-shaped fiber deposition tower 101B in FIG. 12), the supply amount of the material for the absorbent polymer from each polymerization tower (for example, the first polymerization tower 102A and the second polymerization tower 102B in FIG. 12), and the monomer conversion may be suitably selected and determined, and various absorbent composites that differ in point of the morphology of the absorbent composite and of the amount of the absorbent polymer therein may be obtained. Concretely, in general, the unit weight of the fibers to be fed from the non-shaped fiber deposition tower is preferably from 10 to 1000 g/m². In addition, in general, the unit weight of the droplets under polymerization that are fed from the polymerization tower may be preferably from 10 to 500 g/m² in terms of the dry weight of the absorbent polymer measured in determination of the water-holding ability thereof to be mentioned hereinunder.

The second pattern is an absorbent composite in which the fiber layer is relatively thick. This may be produced by increasing the fiber supply amount from the non-shaped fiber deposition tower. In this case, droplets under polymerization are contacted with the relatively thickly-deposited fiber deposition layer to form the absorbent composite. For example, in case where the above-mentioned production device having two-staged non-shaped fiber deposition towers is used (see FIG. 12), the composite produced may have a four-layered laminate structure of fiber deposition layer/absorbent polymer layer/fiber deposition layer/absorbent polymer layer.

The third pattern is an absorbent composite where a fiber deposition layer is laminated on and under an absorbent polymer layer. This may be produced, using the above-mentioned production device that has two-staged non-shaped fiber deposition towers (see FIG. 12) but does not have the latter-stage second polymerization tower and the second surface-treatment agent spray, in which the fiber supply amount in each non-shaped fiber deposition tower is increased.

Apart from the above three patterns, the absorbent composite of the invention may also be produced by opening and sieving the sheet-shaped absorbent composite according to the process mentioned above, then recovering the absorbent polymer and the substrate, laminating them into a sheet through thermal pressing. In this case, the above-mentioned free fibers and/or powdery absorbent polymer may be mixed with the absorbent composite, and may be formed into a sheet. In place of the non-shaped fibers, opened fibers may be supplied from the non-shaped fiber deposition tower, and they may be bound to an absorbent polymer through droplet polymerization. This method is applicable to recycling of the absorbent polymer recovered from the materials broken in the production step, the goods returned from the market owing to soiling or outside the standard, and the used goods.

### IV. Absorbent Composite

The absorbent composite of the invention is characterized in that a bound particulate or web-like absorbent polymer is bound onto a substrate. The ratio by weight of the substrate to the absorbent polymer in the absorbent composite of the invention (this is a ratio by weight of the two after drying in the measurement of the water-holding ability of the composite to be mentioned below) is preferably within a range within which the absorbent composite may satisfy both the essential effect of the substrate such as shape retentive stability and the essential effect of the absorbent polymer such as liquid absorbability. Concretely, the ratio by weight of the substrate to the absorbent polymer is preferably from 1/1 to 1/1,000,000, more preferably from 1/2 to 1/100,000, even more preferably from 1/3 to 1/10,000.

### (Absorption)

The absorbent composite of the invention may absorb liquid either from its substrate side or from its absorbent polymer side. Preferably, the absorbent composite of the invention is hydrophobic on its absorptive side and is hydrophilic on its opposite side. In order that the composite may diffuse the liquid applied thereto, on its absorptive side and that it may rapidly transfer the liquid to the absorbent polymer, it is desirable that the absorbent polymer side of the substrate is hydrophilic and the opposite side thereto is hydrophobic so that the composite may absorb the liquid from its hydrophobic side. It may be considered that the substrate may have an effect of more rapidly absorbing liquid owing to its capillary action and storing it in its voids than the absorbent polymer, and thereafter distributing it to the absorbent polymer.

### (Structure of Absorbent Composite)

In the absorbent composite of the invention, the substrate may be on one side or on both sides of the absorbent polymer. In case where the absorbent polymer is adhesive, sticky or moisture-absorptive and when both sides of the absorbent polymer are covered with a substrate, then it is excellent in point of the line operability thereof since the composite hardly adheres to the contact part and the slide part of the take-up roller, etc. The absorbent composite of the invention may have a multi-layered structure having two or more absorbent polymer layers and/or substrates. In case where the absorbent composite of the invention has plural substrates, then they may be the same or different in point the material and the thickness thereof. The substrate is preferably formed of fluff pulp and/or nonwoven fabric. In case where the composite has plural absorbent polymer layers, then it is desirable that each absorbent polymer layer is bound to the substrate. In case where a substrate is disposed on both sides of the absorbent polymer layer, it is desirable that the absorbent polymer is bound to the substrate on both sides thereof.

The structure having a substrate on both sides thereof may be produced, for example, according to a method (1) where a second substrate is contacted with and bound to the other side of an absorbent polymer that is bound to a first substrate on its one side but is not completely polymerized; or a method (2) where two droplet polymerizations are carried out in parallel, and the surfaces of the absorbent polymers are contacted with each other immediately after their contact with a substrate (in this stage, the polymers are bound to the substrate but are not completely polymerized); or a method (3) where an additional substrate is bound to the side of the absorbent polymer of a composite in which the absorbent polymer is bound to a substrate, by heating it under pressure. Of those, preferred is the method (2) in point of the delamination resistance. In the case of the method where the absorbent polymer is bound to the substrate but is not as yet completely polymerized, the composite may be pressed in such a degree that the absorbent polymer immediately after polymerization is not broken. Concretely, it is desirable that the composite is pressed under a pressure of from 0.0001 to 1 MPa, more preferably from 0.001 to 0.1 MPa. In the method where the absorbent polymer is bound to the substrate but is not as yet completely polymerized, the absorbent polymer is stuck to the substrate by utilizing the adhesive force of the absorbent polymer not as yet completely polymerized and then they are bound to each other while the polymerization is further promoted.

In the production method for the absorbent composite of the invention, it is desirable that the conversion of the polymerizable monomer on the first substrate, which is just contacted with the second substrate, is from 10 to 80%, more preferably from 15 to 65%, even more preferably from 20 to 60%, most preferably from 25 to 55%. The water content of the polymerizable monomer on the first substrate, which is just contacted with the second substrate, is preferably from 25 to 80%, more preferably from 30 to 75%, even more preferably from 35 to 70%, most preferably from 40 to 65%. In order to make the conversion and the water content fall within these ranges, for example, in an atmosphere at from 0 to 40° C., the time to be taken from the dropping of the droplets to the contact thereof with the second substrate may be generally preferably at most 60 seconds, more preferably at most 30 minutes, even more preferably at most 20 minutes.

### (Thickness of Absorbent Composite)

The absorbing speed of the absorbent composite of the invention depends on the type, the thickness and the constitution of each layer constituting it. Of those factors, in particular, the influence of the thickness of the absorbent composite is great. In general, the thickness of the absorbent composite is preferably smaller in point of the adhesiveness between the constitutive layers and of the easiness in producing the capillary phenomenon. On the other hand, however, the thickness is preferably larger in that the physical binding power of the absorbent polymer is smaller. Concretely, for example, the lowermost limit of the thickness of the absorbent composite is preferably 200 μm, more preferably 400 μm, most preferably 600 μm; and the uppermost limit thereof is preferably 10000 μm, more preferably 2500 μm, most preferably 1500 μm. In order that the absorbent composite of the invention may have such a suitable thickness, the composite is preferably shaped under compression.

Preferably, the thickness of the substrate layer is from 0.1 to 100 mm. Also preferably, the lowermost limit of the thickness of the absorbent polymer layer is 50 μm, more preferably 100 μm, even more preferably 200 μm; and the uppermost limit thereof is preferably 3000 μm, more preferably 2000 μm, even more preferably 1000 μm.

### (Absorbing Speed)

The absorbing speed of the absorbent composite of the invention may be generally at least 1 second, preferably at least 10 seconds, and may be generally at most 100 seconds, preferably at most 70 seconds, more preferably at most 50 seconds, even more preferably at most 30 seconds. The preferred absorbing speed of the absorbent composite depends on the use of the absorbent composite and on the constitution of the absorbent article formed with the composite. For example, in case of diapers, the suitable range of the absorbing speed may depend on the liquid dispersiveness and distributiveness in any other material than the substrate and the absorbing polymer. The absorbent composite of the invention may exhibit its preferred absorbing speed especially when it is used in diapers, etc. Not adhering to any theory, the reason for the preference may be because, when the composite is formed into a thin sheet, the substrate on the absorbent polymer side and the substrate on the opposite side may be kept in contact with each other via the pores or the voids in the absorbent polymer, or the two substrates may be close to each other sufficiently in such a degree that they may induce a capillary phenomenon of the absorbed liquid.

The absorbing speed may be determined according to the following manner, depending on the absorbing ability of the absorbent composite at room temperature and room humidity. The room humidity as referred to herein means a humidity of from 30 to 70%. In determining the absorbing speed thereof, an absorbent composite to be analyzed is first cut into a rectangular piece of 40 cm×20 cm. In case where a substrate is formed on both sides of the absorbent polymer, then one substrate is peeled off. On a smooth stainless sheet (thickness 3 mm) of the same size as above, fluff pulp of the same size (unit weight 100 g/m²) is put. On the fluff pulp, the absorbent composite is put with its absorbent polymer side facing downward. On the absorbent composite, a smooth stainless plate (thickness 3 mm) of the same size is put. A load of 10 MPa is applied to the laminate structure in both directions of up and below the stainless plates. This is left at room temperature for 5 minutes, then the pressure is removed, and the absorbent composite is taken out. The center of the resulting absorbent composite is cut into a square of 5 cm×5 cm, and this is dipped in a laboratory dish of glass having a diameter of 10 cm and filled with 25 ml of pure water, whereupon the time taken before a part of the bottom of the dish begins to be exposed out in air (absorbing speed/second) is determined. Thus determined, the time (unit: second) indicates the absorbing speed.

### (Absorption Amount)

When the water-holding ability of the absorbent composite of the invention is evaluated in terms of the absorption amount thereof, then the water-holding ability of the absorbent composite of the invention is especially preferably from 25 to 50%.

### (Absorbing Ability Under Pressure)

Especially preferably, the absorbent composite of the invention has an absorbing ability under pressure, as determined according to the method mentioned hereinunder, of from 10 to 35 under a load of 20 g/cm² applied thereto, and from 8 to 20 under a load of 50 g/cm².

### (Dropout Percentage of Absorbent Polymer)

Especially preferably, the absorbent composite of the invention has an absorbent polymer dropout percentage of at most 30% by weight. The reason may be because, in the absorbent composite of the invention, as raised, the fibers could be buried in or could adhere to the absorbent polymer more deeply or more extensively.

(Gel Dropout Percentage after Absorption)

Especially preferably, the absorbent composite of the invention has a gel dropout percentage of at most 60% by weight, most preferably at most 20% by weight. The gel dropout percentage of the absorbent polymer produced through conventional droplet polymerization is at least 50% by weight. The reason may be because, in the absorbent composite of the invention, as raised, the fibers could be buried in or could adhere to the absorbent polymer more deeply or more extensively. The effect of the raising treatment may be often more remarkable in the reduction in the gel dropout percentage after absorption rather than in the reduction in the absorbent polymer dropout percentage. As compared with the unswollen absorbent polymer, the swollen absorbent polymer may more sensitively reflect a minor difference in the bonding condition thereof on its dropout percentage.

### (Bending Resistance of Absorbent Composite)

Especially preferably, the absorbent composite of the invention has a bending resistance, as measured according to the heart loop method mentioned hereinunder, of at most 4 cm.

### (Others)

The absorbent composite is analyzed in dry for determining the above-mentioned thickness, absorbing speed, absorption amount and absorbent polymer dropout percentage thereof. The absorbent composite is dried until its weight becomes substantially constant (that is, until the water reduction in the absorbent composite or the absorbent polymer could reach at most 5% by weight) under the condition under which the absorbent polymer and the substrate constituting the absorbent composite are not degraded. The drying condition varies depending on the unit weight of the absorbing polymer and that of the substrate. One typical drying condition is, for example, as follows: When an absorbent polymer having a water content of 40% by weight and having a unit weight not more than 300 g/cm² is bound to a nonwoven fabric of PET having a unit weight of 40 g/cm² to constitute a composite, then the composite is dried in a hot air drier at 120° C. for 30 minutes.

### V. Absorbent Article

### (Constitution of Absorbent Article)

The absorbent composite of the invention may be used as a material for various absorbent articles. The constitution of the absorbent article formed with the absorbent composite of the invention may be suitably determined depending on the necessary function and use. Typically, one example is a laminate structure comprising an absorbent article of the invention and a fiber deposition layer. The absorbent composite of the invention may be combined with a material generally used in absorbent articles (e.g., fluff pulp, tissue, nonwoven fabric, polyolefin sheet).

Preferably, the absorbent article of the invention is formed of only the absorbent composite of the invention. However, according to the necessity for its use, free fibers and powdery absorbent polymer may be added to the absorbent composite of the invention not detracting from the excellent advantage of the invention.

Free fibers are fibers not bound to the absorbent polymer. The effect of free fibers may be, for example, the improvement in the flexibility, softness, water conductivity, water permeability, water dispersiveness and air perviousness. When the type of the fibers is suitably selected, then the openability of the absorbent polymer layer may be improved. In case where free fibers are used, concretely, they may be mixed in the absorbent polymer layer in such a manner that the non-shaped fibers could be free fibers.

In case where free fibers are used, fibers of a suitable type, having a suitable length, a suitable linearity and a suitable diameter, are selected appropriately. Regarding the material thereof, the free fibers may be suitably selected from those mentioned hereinabove for the partly-buried substrate and the surface-adhering substrate, from the viewpoint of the above-mentioned various matters. Preferably, the fiber length and the fiber linearity thereof are such that the free fibers could have a suitable degree of mobility so as not to be leaked away from the absorbent polymer layer. From the viewpoint of easy recyclability thereof, it is desirable that the absorbent polymer layer is readily opened. Concretely, the fiber length is preferably from 50 to 100,000 μm, more preferably from 100 to 50,000 μm, even more preferably from 500 to 20,000 μm. The fiber diameter must be such that it may ensure the effect of the free fibers such as the water conductivity and diffusibility thereof. In addition, the thickness of the free fibers is preferably such that the free fibers could prevent their blocking and that they are readily miscible with the absorbent composite and are suitably rigid so that the absorbent composite may be compression-shaped into thin sheets. Especially when the absorbent composite is used for sanitary applications such as sanitary protections, then the thickness of the free fibers is preferably such that they may have a soft feel. Concretely, the fiber diameter is preferably from 0.1 to 500 dtex, more preferably from 0.1 to 100 dtex, even more preferably from 1 to 50 dtex, most preferably from 1 to 10 dtex.

In case where the absorbent article of the invention contains free fibers, the content of the free fibers therein may be to such a degree that the absorbent article containing them may have a suitable bulkiness. Concretely, the ratio by weight of the free fibers to the absorbent composite (the ratio by weight of the two after dried in measurement of the water-holding ability to be mentioned hereinunder) is preferably from 90/10 to 0/100, more preferably from 80/20 to 0/100, even more preferably from 50/50 to 0/100.

In particular, in paper diapers and sanitary napkins, a nonwoven fabric of hydrophobic fibers (e.g., polyethylene fibers, polypropylene fibers, polyester fibers) may be used so as to increase the diffusibility of body discharges through them. The hydrophobic fibers may be mixed with fluff pulp and powdery absorbent polymer.

For the powdery absorbent polymer, usable are various commercially-available powdery products of, for example, polysodium acrylate, polypotassium acrylate, acrylic acid-vinyl alcohol copolymer, crosslinked polysodium acylate, starch-acrylic acid graft copolymer, isobutylene-maleic anhydride copolymer and its saponified derivative. Regarding its absorbing ability, the absorption amount of the absorbent polymer is preferably from 5 to 50 g per gram of the polymer, more preferably from 10 to 50 g, even more preferably from 20 to 50 g. Preferably, the powdery absorbent polymer may be mixed in the absorbent polymer to such a degree that the gel dropout percentage after absorption of the absorbent polymer including the powdery absorbent polymer may be at most 5% by weight. The powdery absorbent polymer may be mixed in any step of producing the absorbent composite and the absorbent article of the invention. For example, it may be previously sprayed onto the substrate before polymerization, or may be added to the polymerization tower during polymerization, or may be added immediately after polymerization. Of those modes, preferred is the mode of adding the powdery absorbent polymer before drying the absorbent polymer, since the powdery absorbent polymer may well adhere to the absorbent polymer and since the absorbent polymer dropout percentage and the gel dropout percentage after absorption may be small.

### (Structure of Diaper)

One typical structure of a diaper is described with reference to FIG. 19. FIG. 19 shows a laminate structure prepared by laminating a tissue 92, an absorbent composite 93, a tissue 94 and a water-pervious fibrous material (e.g., nonwoven fabric of water-pervious polyester) 95 in that order on a water-impervious sheet (e.g., water-impervious polyethylene sheet) 91. These layers are firmly stuck to each other by applying pressure thereto, and then the four sides of the resulting structure are hot-sealed to produce an absorbent article. In this diaper, liquid penetrates from the side of the water-pervious polyester nonwoven fabric 95, and is then absorbed by the absorbent composite 93. On the absorbent composite 93, disposed are fibrous substrates such as the tissue 94 and the water-impervious polyester nonwoven fabric 95. Therefore, in this, liquid may be rapidly absorbed by the absorbent composite, and even though some pressure is applied to the diaper, the absorbed liquid is hardly released out.

In addition, in FIG. 19, a bulky material such as fluff pulp may be inserted into the absorbent article whereby the article may have a softer feel. Preferably, the unit weight of the bulky material usable herein is from 80 to 250 g/m², more preferably from 100 to 220 g/m². Preferably, the bulky material is provided between the absorbent composite 93 and the substrate such as the water-impervious polyethylene sheet 91, but the absorbent composite 93 may be sandwiched between the bulky materials disposed on and below it. In case where the composite is sandwiched between the bulky materials disposed on and below it, it is desirable that the unit weight of the bulky material on the lower side is larger.

### VI. Application

The absorbent article of the invention has many applications. Concretely, for example, it may be suitably used for sanitary materials such as paper diapers for babies, paper diapers for adults, inconsistence pads, sanitary protections; industrial materials such as sheets for absorbing and holding waste water, cut-off materials, sealing materials, dew formation inhibitors for construction; agricultural and horticultural materials such as soil water-holding agents, water-holding sheets for seedlings, freshness-holding agents for vegetables, water-holding agents; civil engineering materials such as mud-adding materials, waste mud-treating agents, low-friction materials; construction materials such as lubricants, void-filling materials; safety materials such as fire extinguishers, refractory materials; distribution materials such as coolants, moisture-controlling agents. Regarding the applications of the absorbent composite of the invention, referred to are Technology and Market of High-Absorbent Resins (Technomart, 1981); High-Absorbent Polymers (Fusayoshi Masuda, Kyoritsu Publishing, 1987); Production and Application of Functional Polymer Gels (Masahiro Irie, CMC, 1987); Development Tendency and Application Prospects of High-Absorbent Resins (Eizo Ohmori, Technoforum, 1987); New Application Development of High-Absorbent Polymers (CMC, 1993); Industrial Materials (Vol. 42, No. 4, 1994); Latest Trend of Polymer Gels (Mitsuhiro Shibayama, Kanji Kajiwara, CMC, 2004); Superabsorbent Polymers Science and Technology (F. L. Bucholz & N. A. Peppas, American Chemical Society, 1993); and Modern Superabsorbent Polymer Technology (F. L. Bucholz & A. T. Graham, Wiley-VCH, 1998).

### EXAMPLES

The characteristics of the invention are described concretely hereinunder with reference to Examples and Comparative Examples given below. In the following Examples, the material used, its amount and ratio, the details of the treatment and the treatment process may be suitably modified or changed not overstepping the sprit and the scope of the invention. Accordingly, the invention should not be limitatively interpreted by the Examples mentioned below.

### Example 1

### Preparation of Starting Materials

573 g of aqueous 48.5 wt. % sodium hydroxide solution, 64 g of water, 1.5 g of a crosslinking agent (N,N′-methylenebisacrylamide) and 50 g of aqueous 30 wt. % hydrogen peroxide solution were added to 1250 g of aqueous 80 wt. % acrylic acid solution to prepare a solution A1. The monomer concentration in the solution A1 was 60% by weight, and the degree of neutralization of the solution was 50 mol %.

573 g of aqueous 48.5 wt. % sodium hydroxide solution, 99 g of water, 1.5 g of a crosslinking agent (N,N′-methylenebisacrylamide) and 15 g of L-ascorbic acid were added to 1250 g of aqueous 80 wt. % acrylic acid solution to prepare a solution B1. The monomer concentration in the solution B1 and the degree of neutralization of the solution were the same as those of the solution A1.

### (Substrate)

An air-through polyester nonwoven fabric comprising linear fibers having a fiber diameter of 6.7 dtex (linear diameter 25 μm), a mean fiber length of 25 mm, an aspect ratio of 1000, a unit weight of 40 g/m², a specific volume of 50 ml/g and a bulk density of 0.02 g/ml was cut into a rectangular piece having a size of 25 cm×50 cm, and this was put on a belt horizontally traveling at a speed of 0.05 m/min by belt conveyor.

In this, the unit weight was determined by cutting the substrate into a square piece having a size of 10 cm×10 cm, and its weight (g) was divided by its area. The specific volume was determined by dividing the volume of the substrate by the weight thereof. The volume of the substrate was determined by measuring the thickness (cm) of a square sample of the substrate having a size of 10 cm×10 cm on its side with a digital photomicroscope followed by multiplying the square of one side (10 cm) of the sample by the thickness thereof.

### (Polymerization)

The solution A1 and the solution B1 were mixed, using a nozzle unit having a structure shown in FIG. 14. The nozzle unit has two supply ducts each for a material solution for an absorbent polymer, and each duct is provided with 5 columnar jet nozzles at intervals of 1 cm (this is the distance between the outer peripheries of the adjacent nozzles). The inner diameter of each nozzle is 0.13 mm. The crossing angle of the facing nozzles is 30 degrees; and the distance between the facing nozzle tips is 4 mm. Both heated at 40° C., the two solutions were jetted out at a flow rate of 5 m/sec with a pump.

The solution A1 and the solution B1 met at around the crossing of the extension lines of the facing nozzles, then formed a column of 10 mm long, and therefore dropped in the vapor phase (in air, normal pressure, open system, temperature 50° C.) as droplets. The diameter of the droplets was 300 μm, and the space density of the droplets was 3 g/m³. At 2 m below the meeting point of the two solutions, the droplets were brought into contact with the horizontally-traveling substrate. The traveling direction of the substrate is vertical to the supply ducts. The water content of the droplets in contact with the substrate was 40% by weight, and the conversion thereof was as in Table 1. The unit weight of the absorbent polymer was 300 g/m², in terms of the weight of the dried absorbent polymer (dry weight thereof in measurement of the water-holding ability mentioned hereinunder).

### (Surface-Crosslinking Treatment)

When the absorbent polymer cooled at 45° C., aqueous 0.5 wt. % glycerin polyglycidyl ether (epoxy equivalent: 145) solution was sprayed thereon at room temperature. The sprayed amount was 500 ppm by weight of the dried absorbent polymer (dry weight thereof in measurement of the water-holding ability mentioned hereinunder). After 1 minute, this was put into an air band drier (where air at 130° C. runs through), and crosslinked. In 2 minutes after introduction into the drier, the water content of the absorbent polymer was 15% by weight. Further, this was kept heated, and when its water content reached 5% by weight, heating it was stopped and this was gradually cooled. The heating time in the drier was 30 minutes in total. The amount of the absorbent polymer adhering to the produced absorbent composite was 300 g per m² of the composite.

### Example 2

### Preparation of Starting Materials

Solutions A2 and B2 were prepared in the same manner as that for the solutions A1 and B1, respectively, in Example 1, for which, however, the amount of the crosslinking agent used was changed to 0.75 g. Solutions A3 and B3 were prepared in the same manner as that for the solutions A1 and B1, respectively, in Example 1, for which, however, the amount of the crosslinking agent used was changed to 3.00 g. The monomer concentration in the solutions A2, A3, B2 and B3, and the degree of neutralization of the solutions were all the same as those of the solutions A1 and B1.

### (Polymerization)

In place of the solution A1 and the solution B1, the solution A2 and the solution B2 were polymerized in the same manner as in Example 1. In this, the same substrate and the same nozzle unit as in Example 1 were used. However, the substrate was put on a belt horizontally traveling at 0.1 m/min by belt conveyor. The diameter of the droplets under polymerization was 300 μm, and the space density of the droplets was 3 g/m³. The water content of the droplets in contact with the substrate (the droplets were brought into contact with the substrate at 2 m below the meeting point of the two solutions) was 40% by weight, the unit weight of the absorbent polymer was 150 g/m², in terms of the weight of the dried absorbent polymer (dry weight thereof in measurement of the water-holding ability mentioned hereinunder), and the conversion in polymerization was as in Table 1.

The thus-obtained precursor composite was immediately put on the belt horizontally traveling by belt conveyor, in place of the substrate. Using this, the solution A3 and the solution B3 were polymerized through the same nozzle unit, in place of the solution A2 and the solution B2. However, the diameter of the droplets under polymerization was 300 μm, and the space density of the droplets was 3 g/m³. The water content of the droplets in contact with the substrate (the droplets were brought into contact with the substrate at 2 m below the meeting point of the two solutions) was 40% by weight, the unit weight of the absorbent polymer was 300 g/m², in terms of the weight of the dried absorbent polymer (dry weight thereof in measurement of the water-holding ability mentioned hereinunder), and the conversion in polymerization was as in Table 1.

The above series of operation was so designed that, in 60 seconds after the mixed droplets of the solution A2 and the solution B2 had been dropped onto the substrate, the mixed droplets of the solution A3 and the solution B3 were dropped onto the substrate (precursor composite). During the process, the substrate was kept in an atmosphere at 50° C.

### (Surface-Crosslinking Treatment)

This was surface-crosslinked in the same manner as in Example 1 to produce an absorbent composite. The heating time in the drier in the surface-crosslinking step was 30 minutes in total. The amount of the absorbent polymer adhering to the surface-crosslinked absorbent composite was 300 g per m² of the composite.

### Example 3

Using the same material solutions and the same substrate as those in Example 2, using the nozzle in the manner mentioned below for polymerization, and effecting the surface-crosslinking treatment as in Example 1, an absorbent composite was produced. Two nozzle units of FIG. 14 were prepared, and they were disposed in parallel to each other on the same face, as spaced by 10 cm in terms of the distance between the center axes of the units. Through one nozzle unit (first nozzle unit), the solutions A2 and the solution B2 were mixed, and at the same time, through the other nozzle unit (second nozzle unit), the solution A3 and the solution B3 were mixed. The droplets were brought into contact with the substrate horizontally traveling at 2 m below the meeting point of the two solutions. The traveling direction of the substrate was vertical to the supply duct of each nozzle unit; and the substrate traveled horizontally first below the first nozzle unit and then below the second nozzle unit. The diameter of the droplets under polymerization was 300 μm, and the space density of the droplets was 6 g/m³. The water content of the droplets in contact with the substrate was 40% by weight, the unit weight of the absorbent polymer was 300 g/m², in terms of the weight of the dried absorbent polymer (dry weight thereof in measurement of the water-holding ability mentioned hereinunder), and the conversion in polymerization was as in Table 1. The heating time in the drier in the surface-crosslinking step was 30 minutes in total. The amount of the absorbent polymer adhering to the surface-crosslinked absorbent composite was 300 g per m² of the composite.

### Example 4

A surface-crosslinked absorbent composite was produced in the same manner as in Example 1, for which, however, the temperature in the vapor phase during polymerization was changed from 50° C. to 40° C.

### Example 5

A surface-crosslinked absorbent composite was produced in the same manner as in Example 2, for which, however, the moving speed of the belt with the substrate put thereon was changed to 0.05 m/min in the first-stage polymerization and to 0.15 m/min in the second-stage polymerization.

### Example 6

An absorbent composite was produced in the same manner as in Example 3, for which, however, the flow rate of the solution A3 and that of the solution B3 were changed to 10 m/sec each, and the moving speed of the belt on the substrate put thereon was changed to 0.075 m/min.

### Example 7

Using a substrate (fluff pulp having a unit weight of 100 g/m²) put on a belt horizontally traveling at 0.10 m/min by belt conveyor, the same polymerizable monomer materials A1 and B1 as in Example 1 were polymerized through droplet polymerization in the same manner as in Example 1, whereby an absorbent polymer was stuck to one surface of the substrate. Two sheets of the substrate having the absorbent polymer adhering on its one surface were prepared, and they were stuck to each other at their absorbent polymer surfaces. This was pressed using Teflon®-coated stainless plates. The time taken after contact with the first substrate and before contact with the second substrate (lamination starting time) was 10 seconds, the temperature in lamination was 50° C., the applied pressure was 0.005 MPa, the conversion of the polymerizable monomer just before lamination with the substrate was 50%, and the water content was 40%. The obtained sample was dried with hot air at 120° C. for 30 minutes, and then cooled to room temperature to produce an absorbent composite. The structure of the cross section of the absorbent composite is as in FIG. 15. Of the properties of the samples evaluated hereinunder, those except the absorbing speed were determined before shaping the samples under compression as in this Example; and the absorbing speed is determined by shaping the non-shaped composite samples as in this Example, under compression according to the method described below, and measuring the thus-shaped samples.

### Example 8

An absorbent composite was produced in the same manner as in Example 7, for which, however, the temperature in the vapor phase during polymerization was changed from 50° C. to 40° C.

### Example 9

In the same manner as in Example 7, a substrate with an absorbent polymer stuck to its one face was prepared. Apart from this, another substrate was prepared in the same manner as herein by adhering an absorbent polymer to one face of the same substrate as in Example 1. The two were stuck to each other at their absorbent polymer surfaces, then pressed and dried in the same manner as in Example 7 to produce an absorbent composite.

### Example 10

In the same manner as in Example 7, three substrates each with an absorbent polymer stuck to one face thereof were prepared. Two of those were stuck to each other at their polymer surfaces. Next, the remaining one was also stuck to it at its polymer surface. In the same manner as in Example 7, this was pressed and dried to produce an absorbent composite. The structure of the cross section of the obtained absorbent composite is as in FIG. 16.

### Example 11

In the same manner as in Example 7, two substrates each with an absorbent polymer stuck to one face thereof were prepared. These were stuck to each other with the polymer side of one substrate facing the substrate side of the other substrate. In the same manner as in Example 7, this was pressed and dried to produce an absorbent composite. The structure of the cross section of the absorbent composite is as in FIG. 17.

### Example 12

An absorbent composite was produced in the same manner as in Example 7, for which, however, the pressure was changed to 0.1 MPa.

### Example 13

An absorbent composite was produced in the same manner as in Example 7, for which, however, a polyester nonwoven fabric (fiber diameter 6.7 dtex (linear diameter, 25 μm), mean fiber length 25 mm, aspect ratio 1000, unit weight 40 g/m², specific volume 50 ml/g, bulk density 0.02 g/ml) was used as the substrate in place of fluff pulp in Example 7.

### Example 14

The same substrate as in Example 1 was cut into a rectangular piece of 25×50 cm. This was kept in a air drier having an inner temperature of 100° C. for 2 minutes and raised. After raised, it had a unit weight of 40 g/m², a specific volume of 100 ml/g, and a bulk density of 0.01 g/ml. The unit weight as referred to herein is obtained by cutting the substrate into a square piece of 10 cm×10 cm, and dividing its weight (g) by its area. The specific volume is obtained by dividing the volume of the substrate by the weight thereof. The volume of the substrate was determined by measuring the thickness (cm) of a square sample of the substrate having a size of 10 cm×10 cm on its side with a digital photomicroscope followed by multiplying the square of one side (10 cm) of the sample by the thickness thereof.

Using the thus-raised substrate and using the same material solutions and the same nozzle unit as in Example 1, an absorbent composite was produced through the same polymerization and the same surface-crosslinking treatment as in Example 1. However, the diameter of the droplets under polymerization was 300 μm, and the space density of the droplets was 3 g/m³. The water content of the droplets in contact with the substrate was 40% by weight. The unit weight of the absorbent polymer was 300 g/m², in terms of the weight of the dried absorbent polymer (dry weight thereof in measurement of the water-holding ability mentioned hereinunder). The monomer conversion was as in Table 1. The heating time in the drier in surface-crosslinking treatment was 30 minutes in total. The amount of the absorbent polymer adhering to the thus-obtained absorbent composite was 1300 g per m² of the composite.

### Example 15

An absorbent composite was produced in the same manner as in Example 14, for which, however, the inner temperature in the air drier in raising treatment was 110° C., the specific volume of the composite having a unit weight of 40 g/m² was 80 ml/g, and the bulk density thereof was 0.0125 g/ml.

### Example 16

An absorbent composite was produced in the same manner as in Example 14, for which, however, the inner temperature in the air drier in raising treatment was 90° C., the specific volume of the composite having a unit weight of 40 g/m² was 70 ml/g, and the bulk density thereof was 0.014 g/ml.

### Example 17

A substrate was prepared in the same manner as in Example 14, for which, however, it was kept in an air drier having an inner temperature of 130° C. for 1 minute in raising it, and therefore the raised substrate had a specific volume of 100 ml/g when its unit weight was 40 g/m², and had a bulk density of 0.001 g/ml. Using it in the same manner as in Example 9, an absorbent composite was produced.

### Example 18

The polyester nonwoven fabric used in Example 13 was raised by keeping it in an air drier having an inner temperature of 130° C. for 1 minute, and the thus-raised fabric had a specific volume of 100 ml/g when its unit weight was 40 g/m², and had a bulk density of 0.001 g/ml. Using the thus-raised polyester nonwoven fabric as the substrate in the same manner as in Example 9, an absorbent composite was produced. The properties of the absorbent composite are shown in Table.

### Example 19

In the apparatus shown in FIG. 12, a two-layered absorbent composite was produced. Using it, a diaper was produced.

### (Preparation of Starting Materials)

33.3 parts by weight of water was added to 100 parts by weight of acrylic acid, and 133.3 parts by weight of aqueous 25 wt. % sodium hydroxide solution was added thereto while cooled at 25° C. or lower, thereby producing an aqueous partially-neutralized acrylic acid solution. To it were added 0.14 parts by weight of a crosslinking agent (N,N′-methylenebisacrylamide) and 4.55 parts by weight of an oxidizing agent (aqueous 31 wt. % hydrogen peroxide solution), to prepare a solution A4. The monomer concentration in the solution A4 was 50% by weight, and the degree of neutralization of the solution was 60 mol %.

0.14 parts by weight of a crosslinking agent (N,N′-methylenebisacrylamide) and 0.57 parts by weight of a reducing agent (L-ascorbic acid) were added to 100 parts by weight of the above aqueous partially-neutralized acrylic acid solution to prepare a solution B4.

### (First-Stage Formation of Substrate)

Pulp fibers having a mean fiber diameter of 2.2 dtex, a mean fiber length of 2,500 μm and a contact angle to water of 0° were fed into the first fiber deposition layer tower 101A along with air thereinto. This was given onto a 250-mesh Teflon net under reduced-pressure suction at −30 mmH₂O from the back of the Teflon net, and deposited thereon at a unit weight of 35 g/m² and a specific volume of 50 ml/g to form a pulp layer. This was conveyed into the first polymerization tower 102A.

### (First-Stage Polymerization)

This was polymerized in the first polymerization tower 102A in the same manner as in Example 1, for which, however, the supply speed of the material solutions for the absorbent polymer and the height of the nozzle unit were changed. The solution A4 and the solution B4 were fed with a pump at a flow rate of 13 g/min each. The nozzle unit was so disposed that the nozzle tip could be at 2.5 m above the substrate. The diameter of the droplets, the space density of the droplets and the water content of the droplets in contact with the substrate were the same as in Example 1. The conversion of the droplets in contact with the substrate was 60%. The unit weight of the absorbent polymer was 200 g/m², in terms of the weight of the dried absorbent polymer (dry weight thereof in measurement of the water-holding ability mentioned hereinunder). The sheet thus obtained herein was conveyed to the first surface-treatment agent spray 103A.

### (First-Stage Surface-Crosslinking Treatment)

The sheet conveyed from the first polymerization tower 102A was sprayed with an aqueous 3 wt. % diethylene glycol diglycidyl ether solution from the first surface-treatment agent spray 103A, at room temperature. The spraying amount was 1000 ppm by weight of the dried absorbent polymer (dry weight thereof in measurement of the water-holding ability mentioned hereinunder). The sprayed sheet was conveyed to the second fiber deposition layer tower 101B.

### (Second-Stage Formation of Substrate)

The same pulp fibers as in the first stage were deposited onto the sheet conveyed from the surface-treatment agent spray 103A, in the same manner as in the first stage but in the second fiber deposition layer tower 101B, thereby forming a pulp layer. The unit weight of the pulp layer was 15 g/m². The obtained sheet was conveyed to the second polymerization tower 102B.

### (Second-Stage Polymerization)

Under the same condition as in the first stage, the sheet conveyed from the second fiber deposition layer tower 101B was polymerized in the second polymerization tower 102B. The diameter of the droplets, the space density of the droplets, and the water content of the droplets in contact with the substrate were the same as those in Example 1. The conversion of the droplets in contact with the substrate was 60%. The unit weight of the thus-polymerized absorbent polymer was 100 g/m², in terms of the weight of the dried absorbent polymer (dry weight thereof in measurement of the water-holding ability mentioned hereinunder). The obtained sheet was conveyed into the second surface-treatment agent spray 103B.

### (Second-Stage Surface-Crosslinking Treatment)

In the same manner as in the first stage, aqueous 3 wt. % diethylene glycol diglycidyl ether solution was sprayed onto the sheet conveyed from the second polymerization tower 102B, in the second surface-treatment agent spray 103B. Heating the substrate was stopped when the water content thereof reached 5% by weight, and this was gradually cooled and then further dried in a drier. The amount of the absorbent polymer adhering to the dried product was 300 g per m² of the product.

### (Drying Step)

The sprayed sheet was put in an air band drier (where air at 120° C. runs through), and dried with hot air for 30 minutes to obtain an absorbent composite. After dried, the water content of the absorbent polymer in the absorbent composite was 5% by weight. The ratio by weight of the absorbent polymer to the pulp in the absorbent composite was 86/14, and the unit weight of the composite as the total of the two was 350 g/m². The unit weight of the absorbent polymer therein was 300 g/m².

The surface of the absorbent composite was observed with a microscope. As a result, it was confirmed that a bound particulate absorbent polymer of two or more nearly-spherical absorbent polymer particles bound to each other, and a web-like absorbent polymer were bound to the substrate. In addition, surface-adhering fibers and partly-buried fibers were also confirmed (see FIG. 18).

### (Production of Absorbent Article)

Using the absorbent composite, a diaper having a layer constitution of FIG. 19 was produced. The absorbent composite was cut into a rectangular piece having a size of 40 cm×10 cm. A water-impervious polyethylene sheet (unit weight 18 g/m²), tissue (unit weight 14 g/m²), and a water-pervious polyester fiber nonwoven fabric (unit weight 23 g/m²) were cut into rectangular pieces each having a size of 40.5 cm×10.5 cm. On the water-impervious polyethylene sheet 91, laminated were the tissue 92, the absorbent composite 93, the tissue 94, and the water-impervious polyester fiber nonwoven fabric 95 in that order. The laminate were sandwiched between smooth stainless plates (thickness 3 mm) put on and below it, and a load of 0.4 MPa was applied to it. After left as such for 20 minutes, the pressure was removed and the laminate was taken out. The four sides of the laminate were hot-sealed. The outer edges of the sealed part were cut off, and an absorbent article having a size of 40 cm×10 cm was thus obtained.

### Example 20

A single-layered absorbent composite was produced. Concretely, the absorbent composite was produced in the same manner as in Example 19, for which, however, the second-stage treatment (substrate formation, polymerization for absorbent polymer layer, and surface-crosslinking treatment) was not carried out.

### Example 21

An absorbent composite was produced, in which the fibers in the first-stage substrate differ from those in the second-stage substrate. Concretely, the absorbent composite was produced in the same manner as in Example 19, for which, however, pulp fibers were changed to polyester fibers (mean fiber length 600 μm, mean fiber diameter 1.5 dtex, contact angle to water 80°) for the substrate used in the second stage in Example 19, and the fibers were deposited to give a unit weight of 15 g/m².

### Example 22

An absorbent composite having free fibers was produced. Concretely, the absorbent composite produced in Example 19 was opened with an opening device. To this were added free fibers (of the same pulp as in Example 19) so that the pulp ratio could be 25% by weight. This was uniformly mixed, and compactly laid on a stainless plate to give a unit weight of the absorbent polymer of 200 g/m². A stainless plate was put on it, and a pressure of 10 MPa was applied thereto at 25° C. for 20 minutes. The pressure was removed, and a high-density absorbent composite was taken out.

### Example 23

Using a powder absorbent polymer, an absorbent composite was produced. Using it, a diaper was produced. Concretely, the absorbent composite produced in Example 20 was opened with an opening device. To this was added a powdery absorbent polymer (Sandia Polymer's Aquapearl DS51) so that the proportion of the absorbent polymer could be 86% by weight. This was uniformly mixed, and compactly laid on a stainless plate to give a unit weight of the absorbent polymer of 300 g/m². A stainless plate was put on it, and a pressure of 10 MPa was applied thereto at 25° C. for 20 minutes. The pressure was removed, and a high-density absorbent composite was taken out. Using the thus-obtained absorbent composite, a diaper was produced in the same manner as in Example 19.

### Example 24

To 125 parts by weight of aqueous 80 wt. % acrylic acid solution, added were 57.3 parts by weight of aqueous 48.5 wt. % sodium hydroxide solution, 6.4 parts by weight of water, 0.15 parts by weight of a crosslinking agent, N,N-methylenebisacrylamide, and 5.0 parts by weight of an oxidizing agent, aqueous 30 wt. % hydrogen peroxide solution to prepare a solution A5. The monomer concentration in the solution A5 was 60% by weight; and the degree of neutralization of the solution was 50 mol %.

Apart from it, a solution B5 was prepared by adding 57.3 parts by weight of aqueous 48.5% by weight of sodium hydroxide solution, 9.9 parts by weight of water, 0.15 parts by weight of a crosslinking agent, N,N-methylenebisacrylamide, and 1.5 parts by weight of a reducing agent, L-ascorbic acid to 125 parts by weight of aqueous 80 wt. % acrylic acid solution. The monomer concentration in the solution B5 was 60% by weight; and the degree of neutralization of the solution was 50 mol %.

Using a double concentric centrifugal jet nozzle having a horizontal cross section shown in FIG. 4(a) and having a vertical cross section shown in FIG. 13(c), the monomer solutions A5 and B5 were fed to the inner jet-out port and the outer jet-out port, respectively. The liquid temperature of each solution was 40° C., and the solutions were jetted out at a flow rate of 400 ml/min with a pump. The diameter of the nozzle constituting the inner jet-out port of the double concentric centrifugal jet nozzle was 1.0 mm, the outer diameter thereof was 1.7 mm; the inner diameter of the nozzle constituting the outer jet-out pot was 2.5 mm, the outer diameter thereof was 3.0 mm; and the wall thickness of the nozzle tip constituting the outer jet-out port was 0.25 mm. As in FIG. 13(c), the double concentric centrifugal jet nozzle was tapered in the direction toward the nozzle tip, and the crossing angle of the taper was 120°. Onto the outer wall surface of the taper part of the double concentric centrifugal jet nozzle, sprayed was a silicon oil protective film to form therein a protective liquid film having a thickness of 0.5 mm, as in FIG. 1.

The solution A5 and the solution B5 collided with each other at around the outlet of the nozzle, and were thus pulverized to give droplets, and while polymerized, they dropped down in a vapor phase (in air, at a temperature of 50° C.). A part of the droplets collided with each other in the vapor phase to form aggregated particles, and dropped down on a polyester nonwoven fabric (unit weight: 30 g/m²) disposed at 3 m below the tip of the jet-out port of the nozzle, and were completely polymerized on it. This was dried at 110° C. for 1 hour to obtain an absorbent composite.

Under the condition as above, the device was driven continuously for 3 hours, and then the tip of the double concentric centrifugal jet nozzle was checked as to whether or not some particles adhered thereto. As a result, adhesion of no particles was found.

### Example 25

An absorbent composite was produced in the same manner as in Example 24, for which, however, the inner diameter of the nozzle constituting the inner jet-out port of the double concentric centrifugal jet nozzle was changed to 0.9 mm, the inner diameter of the nozzle constituting the outer jet-out port was to 2.8 mm, and the wall thickness of the nozzle tip constituting the outer jet-our port was to 0.1 mm, and the monomer solutions A5 and B5 were jetted out at a flow rate of 500 ml/min.

Under the condition as above, the device was driven continuously for 3 hours, and then the tip of the double concentric centrifugal jet nozzle was checked as to whether or not some particles adhered thereto. As a result, adhesion of no particles was found.

### Example 26

Using a double concentric centrifugal jet nozzle shown in FIG. 4 (in which, however, a protective liquid was jetted out through a protective liquid jet-out slit, as in FIG. 2), the monomer solutions A5 and B5 in Example 24 were fed to the first nozzle 10 and the second nozzle 20, respectively, and polymerized. The inner diameter of the nozzle orifice 10A of the first nozzle 10 of the centrifugal jet nozzle was 1.0 mm, and the inner diameter of the nozzle orifice 20A of the second nozzle 20 was 2.5 mm. The liquid temperature of each solution was 40° C. Using a pump, the solution A5 was jetted out through the first nozzle 10 at a flow rate of 300 ml/min and at a jet-out pressure of 0.5 MPa; and the solution B5 was jetted out through the second nozzle 20 at a flow rate of 300 ml/min and at a jet-out pressure of 0.2 MPa. Simultaneously with the solutions A5 and B5, a protective liquid, water was continuously jetted out through the protective liquid jet-out slit 4, thereby forming a protective liquid film having a thickness of 0.5 mm on the outer wall surface of the nozzle that covers the lower small-diameter cylindrical part and the taper part of the nozzle. The thickness of the protective liquid film was determined by dividing the supply amount of the protective liquid by the protective liquid film-forming area. The protective liquid supply amount (jet-out flow amount) was 500 ml/min per m² of the protective liquid film-forming area of the nozzle, and even though the protective liquid ran down through the slit to reach the jet-out port, it did not disturb the jetted-out liquid film formed by the solutions A5 and B5.

The solution A5 and the solution B5 collided with each other at around the outlet port of the nozzle, and were pulverized into droplets, and while polymerized, they dropped down in a vapor phase (in air, at a temperature of 50° C.). A part of the droplets collided with each other in the vapor phase to form aggregated particles, and dropped down on a polyester nonwoven fabric (unit weight: 30 g/m²) disposed at 3 m below the tip of the jet-out port of the nozzle, and were completely polymerized on it, thereby forming an absorbent composite. The absorbent composite was dried at 110° C. for 1 hour.

### Comparative Example 1

An absorbent composite was produced in the same manner as in Example 1, for which, however, the distance between the meeting point of the two solutions for polymerization and the substrate was changed from 2 m to 3 m.

### Comparative Example 2

In the same manner as in Example 7, an absorbent polymer was adhered to one surface of a substrate, for which, however, the temperature in the vapor phase during polymerization was changed to 90° C. Two substrates were stuck together at their absorbent polymer surfaces, and pressed using Teflon®-coated stainless plates. In this, the time taken after contact with the first substrate and before contact with the second substrate (lamination starting time) was 3600 seconds, the temperature in lamination was 50° C., the applied pressure was 0.005 MPa, the conversion of the polymerizable monomer just before lamination with the substrate was 85%, and the water content was 15%. In the same manner as in Example 7, the obtained sample was dried with hot air, and then cooled to room temperature to produce an absorbent composite.

### Comparative Example 3

In the same manner as in Example 7, an absorbent polymer was adhered to one surface of a substrate. Two substrates were stuck together at their absorbent polymer surfaces, and pressed using Teflon®-coated stainless plates. In this, the time taken after contact with the first substrate and before contact with the second substrate (lamination starting time) was 20 seconds, and the temperature in lamination and the applied pressure were the same as in Example 7. The conversion of the polymerizable monomer just before lamination with the substrate was 50%. The water content was controlled to be 90% by spraying pure water at room temperature onto the absorbent polymer while laminated with the substrate. In the same manner as in Example 7, the obtained sample was dried with hot air, and then cooled to room temperature to produce an absorbent composite.

### Comparative Example 4

In the same manner as in Example 7, an absorbent polymer was adhered to one surface of a substrate, for which, however, the temperature in the vapor phase during polymerization was changed to 5° C. Two substrates were stuck together at their absorbent polymer surfaces, and pressed using Teflon®-coated stainless plates. In this, the time taken after contact with the first substrate and before contact with the second substrate (lamination starting time), the temperature in lamination, and the applied pressure were the same as in Example 7. The conversion of the polymerizable monomer just before lamination with the substrate was 8%, and the water content was 46%. In the same manner as in Example 7, the obtained sample was dried with hot air, and then cooled to room temperature to produce an absorbent composite.

### Comparative Example 5

An absorbent composite having a web-like absorbent polymer alone was produced. Concretely, the absorbent composite was produced in the same manner as in Example 20, for which, however, the nozzle unit was so disposed in the polymerization tower that the nozzle tip could be at 0.8 m above the substrate. The absorbent composite was observed with a microscope, in which the absorbent polymer expanded like a web. The web-like absorbent polymer particles were linked together via the fibers therebetween, and formed a network structure.

### Comparative Example 6

To an aqueous monomer solution comprising 70 mol % of sodium acrylate and 30 mol % of acrylic acid (the monomer content was 42% by weight in total), added was a crosslinking agent, tetraethylene glycol diacrylate (To a Gosei's Aronix M-240) in an amount of 0.05% by weight based on the monomer weight. The aqueous monomer solution was cooled to 20° C. Nitrogen gas was introduced into it so that the dissolved oxygen concentration in this was reduced to at most 1 ppm. To this were added a photopolymerization initiator, 1-hydroxycyclohexyl phenyl ketone in an amount of 0.02% by mass based on the monomer mass, and a thermal polymerization initiator, sodium persulfate in an amount of 0.15% by mass based on the monomer mass.

An air-through nonwoven fabric of polyethylene/polypropylene (specific volume 50 cm³/g, unit weight 40 g/m²) was heated in an oven at 110° C. for 3 hours. The specific volume of the nonwoven fabric changed to 100 cm³/g. The above aqueous monomer solution was sprayed onto the nonwoven fabric, through a spray nozzle. Its coating amount was 238 g/m². In a nitrogen atmosphere, this was irradiated with UV rays (the UV-ray dose was 2500 mJ/cm²), using a high-pressure mercury lamp. This was put into an air band drier where air runs at 130° C., and when the water content of the absorbent polymer reached 5% by weight, heating this was stopped and this was then gradually cooled. In the thus-obtained absorbent composite, fine particulate absorbent polymer particles adhered to the fibers like bonded beads, and the individual particles existed independently of each other.

The absorbent polymer particles adhered to the obtained absorbent composite in an amount of 100 g/m².

### Comparative Example 7

The same process as in Comparative Example 6 was carried out, in which, however, the coating amount of the aqueous monomer solution was 714 g/m². The obtained absorbent composite was not soft, in which most absorbent polymer had a web-like structure and few polymer particles bonded like beads. The absorbent polymer adhered to the absorbent composite in an amount of 300 g/m².

### <Methods of Measurement and Methods of Evaluation>

Methods for measurement of the data given in the above Examples and Comparative Examples; and methods for evaluation of the performance of the absorbent composites and the diapers produced in the above Examples and Comparative Examples are described below. The results of measurement and performance evaluation are shown in Table 1.

### (Determination of Diameter of Droplets in Droplet Polymerization)

The diameter dd of the droplets in droplet polymerization was calculated according to the following formula, based on the mean diameter dp of the absorbent polymer mass to constitute an absorbent composite and the monomer concentration (total concentration of acrylic acid and sodium acrylate) Cm:

dd=dp/(Cm)^{1/3}.

### (Determination of Contact Angle of Substrate)

The contact angle of the substrate was measured with an automatic contact angle gauge, Kyowa Kaimen Kagaku's CA-V Model. First, the substrate to be tested was dissolved or dispersed in a solvent to prepare a solution having a concentration of from 1 to 10% by weight. The solution was poured into a laboratory dish and expanded thin. Next, the solvent was gradually evaporated away at room temperature, using dry air. The contact angle of the resulting film to distilled water was measured at room temperature. In Examples 19, 20, 22 and 23, water was used as the solvent; and in Example 21, water and tetrahydrofuran were used.

### (Determination of Profile of Absorbent Composite)

The absorbent composites of Examples 1 to 6 were observed with a scanning electronic microscope (SEM). Their pictures are in FIGS. 5 to 10. These picture confirm a structure that comprises, bound to a substrate, a web-like absorbent polymer and a bound particulate absorbent polymer of nearly spherical absorbent particles bound to each other.

The absorbent composite was cut into a piece having a size of 5 cm×5 cm square. Its cross section was photographed with a digital photomicroscope, Keyence's VH-8000 (FIG. 20 shows a picture of the absorbent composite of Example 1). According to JIS 1-1096, the thickness of five substrates and the thickness of the absorbent polymer layer were measured, and their mean value was obtained (see FIG. 21)

(a) An adaptor 26 having a diameter of 30 mm was fitted to a rheometer (FUDOH's Model, NRM-2003J).

(b) An absorbent composite 28 was put on a sample bed 27. The sample bed 27 was elevated at a speed of 2 cm/min, and stopped when a pressure of 0.2 psi was given thereto. Using vernier calipers, the distance t between the upper face of the adaptor 26 and the lower face of the stopped sample bed 27 was measured.

(c) With no sample put thereon, the sample bed 27 was elevated at a speed of 2 cm/min, and stopped when a pressure of 0.2 psi was given thereto. Using vernier calipers, the distance t₀ between the upper face of the adaptor 26 and the lower face of the stopped sample bed 27 was measured.

(d) The thickness of the sample was computed according to the following formula:

Thickness (mm)=(found value t (mm) of the sample)−(found value t₀ (mm) of the blank).

### (Determination of Bulk Density)

An absorbent composite was cut into a piece having a size of 5 cm×5 cm square, and its weight was measured. According to the following formula, its bulk density was obtained. Five samples were tested, and their data were averaged.

Bulk Density (g/cm³)=[weight (g)/thickness (cm)×area (cm²)].

### (Determination of Profile of Absorbent Polymer)

An absorbent composite was cut into a piece having a size of 5 cm×5 cm square. Using a stainless double-edged knee-shaped clipper scissors (FST 14063-09), the substrate was removed. Using the scissors, the absorbent polymer was cut and divided into the bound particulate polymer and the web-like polymer while carefully observed with a digital photomicroscope (Keyence's VH-8000, having a magnification of from 25 to 150). The weight of each part was measured, and the ratio by weight of the bound particulate polymer to the web-like polymer was computed.

Using SEM, a digital photomicroscope and a dial gauge, the profile of the absorbent polymer was analyzed.

With the naked eye or with a digital photomicroscope having a magnification of from 25 to 175, the profile of the bound particulate mass was observed; and using a dial gauge, the major diameter and the thickness of the mass, the number of the particles constituting the mass and the fiber length in the mass were measured. Using SEM and a digital photomicroscope, the diameter of 10 primary particles the absorbent polymer randomly selected from at least 50 bound particulate absorbent polymer particles was measured, and the data were averaged.

Using a digital photomicroscope, the web-like polymer was observed. On the substrate, the area where web-like polymer particles spotwise exist was judged as “web-like dispersion layer”, and the area where web-like polymer particles continue with some pores spotwise existing somewhere among them was judged as “web-like continuous layer”.

The thickness of randomly selected 10 of at least 50 web-like dispersion layers was measured with a dial gauge, and their mean value is referred to as the thickness of the web-like dispersion layer. In a scanning electromicroscopic picture of the web-like dispersion layer, the mean minor diameter and the mean major diameter of randomly selected 10 of at least 50 layers are referred to as a minor diameter and the major diameter, respectively.

The thickness of randomly selected 10 of at least 50 web-like continuous layers was measured with a dial gauge, and their mean value is referred to as the thickness of the web-like continuous layer. In a scanning electromicroscopic picture of the web-like continuous layer, the mean minor diameter and the mean major diameter of randomly selected 10 of at least 50 pores are referred to as a minor diameter and the major diameter, respectively, of the pores. The porosity is the percentage of the overall area of the pores in 25 cm² of the web-like continuous layer.

### (Determination of Conversion)

The conversion in polymerization at a site below the meeting point of two material solutions by their dropping distance was determined according to the following process.

A beaker filled with 150 g of methanol was set so that the methanol surface could be at the site below the meeting point of two material solutions by their dropping distance. One g of a reactant liquid was put into methanol, and the monomer amount per gram of the reactant liquid was determined through liquid chromatography. The methanol solution was dried under pressure at 130° C. for 3 hours, and the polymer weight in the liquid was determined. From each weight, the conversion was computed according to the following formula (where Mp indicates the polymer weight, and Mm indicates the monomer weight). For the liquid chromatography, used was Shimadzu's column shim-pack SCR-1001H built-in LC-10AS/SPD-10A.

Conversion (%)=[Mp/(Mm+Mp)]×100.

(Determination of Conversion in Structure with Substrate on Both Sides of Absorbent Polymer)

An absorbent composite was cut into a piece having a size of 5 cm×5 cm square. This was dipped in 150 g of methanol at room temperature for 12 hours so that the monomer and others were fully released out. Then, this was dried under reduced pressure, using a rotary oil pump at 110° C. for 3 hours, and then its weight was measured, Mc. The monomer amount in methanol was obtained through liquid chromatography, and its weight was Mm. For the liquid chromatography, used was Shimadzu's column shim-pack SCR-1001H built-in LC-10AS/SPD-10A.

On the other hand, only the same substrate as that used in the absorbent composite was processed in the same manner as above and its weight was Ms. From the weight data, the conversion was computed according to the following formula:

Conversion (%)=[(Mc−Ms)/(Mm+Mc−Ms)]×100.

### (Determination of Water Content)

The water content at the site below the meeting point of two material solutions by the dropping distance of the droplets was determined according to the following process.

An absorbent composite was cut into a piece having a size of 7 cm×7 cm square. Using an IR water gauge (IR water gauge by Kett Electric Laboratory, FD-100 (drying heat source: 280 W cyclic ceramic-coated sheath heater)), this was heated at 110° C. for 30 minutes, and then weighed. The weight of the absorbent composite before heating was W1, and the weight thereof after heating was W2. Only the same substrate used in the absorbent composite was processed in the same manner, and its weight was W₀. From the weight data, the water content (%) was computed according to the following formula:

Water Content (%)=[(W1−W0)/(W2−W0)]×100.

### (Determination of Water-Holding Ability (CRC))

An absorbent composite was dried until its weight change could be at most 1% when dried at 110° C. for 30 minutes (this is the weight of the above-mentioned dried absorbent polymer). Then, this was cut into a piece in which the weight of the absorbent polymer is 1 g (the weight of the absorbent composite is W₁.) This was put into a 250-mesh nylon bag (having a size of 20 cm×10 cm), and dipped in from 500 to 1000 ml of physiological saline (0.9% by weight) at 25° C. along with the bag therein, for 30 minutes. The nylon bag was pulled up, hung as it was for 15 minutes to remove water, and then dewatered under 90 G for 90 seconds, using a centrifuge (its weight inclusive of nylon bag is W₃).

On the other hand, only the same substrate as that used in the absorbent composite was processed in the same manner, and cut into a piece having a weight of 1 g. Its weight was W₂; and after processed in physiological saline, its weight inclusive of nylon bag was W₄. From the weight data, the water-holding ability of the sample was computed according to the following formula. The unit of W₁ to W₄ is all gram (g).

S=[(W3−W4)/(W1−W2)].

### (Determination of Absorbent Capacity Under Pressure (AUL)

Using a device shown in FIG. 22, the absorbent capacity under pressure was determined according to the following process. The device for measurement comprises a metal cylinder 46 (inner diameter 25.4 mmφ) of which the bottom is closed with a metal net (#100); a columnar weight 48 slightly smaller than the inner diameter of the cylinder; and a laboratory dish 47.

(a) Under a Load of 20 g/cm² (Using Weight of 100 Gas the Columnar Weight 48):

1) An absorbent composite was dried until its weight change could be at most 1% when dried at 110° C. for 30 minutes. This was blanked out with a blanking punch into a sample disc 45 (25 mmφ).

2) The weight Sd (g) of the sample disc 45, and the weight Td (g) of the metal net-fitted cylinder 46 were measured.

3) 25 g of the following artificial urine at 25° C. was put into the dish 47 (100 mmφ).

4) The sample disc 45 was put into the cylinder 46 with its substrate side facing downward.

5) The columnar weight 48 was laid on the sample disc 45 in such a manner that the columnar weight 48 could not be in contact with the cylinder 46 to generate friction owing to the elevation of the weight through absorption.

6) The cylinder 46 with the sample disc 45 and the columnar weight 48 therein was gently dipped in the dish 47 with the metal net side facing downward.

7) This was statically left as such for 1 hour for absorption.

8) The cylinder 46 was gently taken out from the dish 47.

9) The cylinder 46 was gently put on filter paper (#424), and water adhering to the cylinder 46 was wiped away.

10) The columnar weight 48 was removed (the absorbent polymer adhering to the columnar weight 48 was transferred to the cylinder 46).

11) The weight Tw (g) of the cylinder 46 was measured.

12) The weight Sw (g) of the absorbed sample disc 45 was obtained according to the following formula:

Sw=Tw−Td.

13) A control sample of only the same substrate as that used in the absorbent composite in place of the absorbent composite was used in place of the sample disc in the above operation, and its weight Nd (g) and the weight Mw (g) of the absorbed cylinder were measured. The weight Nw (g) of the absorbed disc was obtained according to the following formula:

Nw=Mw−Td.

14) The absorbent capacity under pressure was computed according to the following formula:

Absorbent Capacity under Pressure=(Sw−Nw)/(Sd−Nd).

The artificial urine has the following composition:

| | | |
|---|---|---|
| Urea | 19.4 | g |
| Sodium chloride | 8.0 | g |
| Anhydrous calcium chloride | 0.6 | g |
| Magnesium sulfate 7-hydrate | 2.05 | g |
| Pure water | 970.9 | g |

(b) Under a Load of 50 g/cm²:

The absorbent capacity under pressure was determined in the same manner as in (a), for which, however, a weight of 250 g was used as the columnar weight 48.

### (Determination of Absorbent Polymer Dropout Percentage)

(a) An absorbent article was cut into a piece having a size of 10 cm×10 cm. Its weight was measured, and from the constitution (the unit weight of the absorbent polymer) of the absorbent composite, the estimated weight of the absorbent polymer is computed. At the center of a standard sieve 61 defined by JIS Z8801 (the dimension of the inner frame is such that the inner diameter is 150 mm and the depth is 45 mm, and this is 20-mesh), the absorbent composite 60 was fixed with a tape 62 (see FIG. 23).

(b) The standard sieve with the absorbent composite fixed thereon, was fixed on the outermost stage of a ro-tap shaker 65 (Tokyo Shinohara Seisakusho's Model SS-S-228, based on JIS Z8815—see FIG. 24).

(c) The shaker 65 was shaken for 60 minutes, set at an impulsion of 165 strokes/min and at a revolution of 290 rpm. The weight of the absorbent polymer dropped from the absorbent composite 60 was measured, and the dropout percentage was obtained according to the following formula:

Dropout Percentage (%)=[(weight of the dropped absorbent polymer (g))/(estimated weight of all absorbent polymer (g)]×100.

(Determination of Gel Dropout Percentage of Absorbent Composite)

The absorbent gel dropout percentage of an absorbent composite, to which a rubbing force was applied, was measured according to the following process (see FIG. 25).

(a) An absorbent composite 52 was put on a smooth face 51. An acrylic plate 55 (rectangular parallelepiped of 100 mm×100 mm×10 mm) was put on it. At the center of the acrylic plate 55, a cylinder 53 opened at its top and having an inner diameter of 40 mm was put, and 7 through-holes 54 each having a diameter of 5 mm were formed at nearly regular intervals in the area surrounded by the cylinder. The mass of the acrylic plate 55 was 150 g inclusive of the cylinder 53.

(b) 150 ml of artificial urine was put into the cylinder 53 and absorbed by the absorbent composite.

(c) After having completely absorbed it, the composite was left at room temperature for 30 minutes.

(d) A center part of an absorbent composite 70 was cut into a square having a size of 10 cm×10 cm, and its mass was measured (see FIG. 26).

(e) The thus-cut sample 73 was put at the center of an acrylic plate 74 having a size of 20 cm×20 cm. A weight 75 (3 kg) having the same size as that of the sample 73 (square of 10 cm×10 cm) was laid on it (see FIG. 27).

(f) The acrylic plate 74 with the sample 73 and the weight 75 put thereon was set in a shaker (Inai Seiei-do's Model MS-1) in such a manner that the cut section of the sample could be vertical to the shaking direction of the shaker, and it was shaken for 30 minutes at an amplitude of 50 mm and an impulsion of 80 strokes/min.

(g) The weight was removed, and the mass of the absorbent gel dropped out from the sample was measured. According to the following formula, the gel dropout percentage of the absorbent composite was computed.

Gel Dropout Percentage (%) of Absorbent Composite=[(amount of extruded gel (g))/(amount of gel before extrusion (g))]×100.

(Determination of Gel Dropout Percentage after Absorption)

The weight of the absorbent polymer gel dropped out from the absorbent composite after determination of the water-holding ability thereof and the weight of the absorbent polymer gel not dropped out were measured. From the weight data, the gel dropout percentage was computed according to the formula mentioned below. Concretely, the absorbent composite processed for measurement of its water-holding ability was gently taken out of the nylon bag. At this time, the absorbent polymer dropped out from the composite to remain in the bag is a dropout absorbent polymer gel. The weight of the bag was subtracted from the weight of the sample inclusive of the bag, and this is the weight Wa (unit, g) of the dropped absorbent polymer gel. The absorbent polymer not dropped from the composite is a non-dropped absorbent polymer gel. The value obtained by subtracting the weight of the substrate from the weight of the composite is the weight Wb (unit, g) of the non-dropped absorbent polymer gel.

Gel Dropout Percentage (%) after Absorption=[Wa/(Wa+Wb)]×100.

### (Determination of Absorbing Speed)

An absorbent composite was cut into a rectangular piece having a size of 40 cm×20 cm. Between two smooth stainless plates having the same size (thickness, 3 mm), sandwiched was fluff pulp (Warehouser's NB-416, having a unit weight of 100 g/m²) having the same size and the absorbent composite laid on it (in such a manner that the absorbent polymer side could face inside). A load of 10 MPa was applied to the laminate on both sides of the stainless plates. This was left at room temperature for 5 minutes, and then the absorbent composite was taken out. The center of the resulting absorbent composite was cut into a square sample of 5 cm×5 cm, and this was dipped in 25 ml of pure water put in a laboratory dish of glass having a diameter of 10 cm. The time taken before a part of the bottom of the dish began to be exposed out in air (absorbing speed/second) was measured. This was measured at room temperature and room humidity, and the measured time is the absorbing speed (unit: second).

The cross section of the absorbent article of Example 2 is shown in FIG. 28. FIGS. 20, 29 and 30 are the digital photomicroscopic pictures of the cross section of the absorbent composites of Examples 1 to 3.

### (Determination of Bending Resistance of Absorbent Composite)

An absorbent composite was cut into a rectangular piece of 2 cm×25 cm. This was kept for one full day at a temperature of 25° C. and a humidity of 50%, and then according to JIS L-1096 (heart loop method for relatively soft fabric), the bending resistance of the sample was determined according to the process mentioned below (see FIG. 31). A larger value found means that the sample is softer.

(a) A sample piece 42 was heart-loopwise fitted to the grip 41 of a horizontal bar so that the effective length of the sample piece 42 could be 20 cm.

(b) After 1 minute, the distance L (cm) between the top of the horizontal bar and the lowermost point of the loop was measured. Five sample pieces were tested, and their mean value was obtained. This is the bending resistance of the sample.

The sample of the absorbent composite of Comparative Example 1 was broken during the test, and its bending resistance could not be determined.

### (Determination of Openability of Absorbent Composite)

The weight K1 (unit, g) of an absorbent composite was measured. The absorbent composite was forcedly opened, and sieved through a screening device (see FIG. 32 (a), (b)). The opening device 151 comprises acrylic cylinders 151A and 151B having an outer diameter of 5 cm and a length of 20 cm, and stainless pins 152 having a thickness of 1 mm and a length of 1 cm are planted on the surfaces of the cylinders at intervals of 5 mm. The cylinder 151A rotates at a revolution of 500 rpm while the cylinder 151B rotates at a revolution of 900 rpm, both in the same direction. The screening device 153 comprises an acrylic cylinder 154 having an inner diameter of 9 cm, which is provided with a stirring blade 155 having an outer diameter of 8 cm and a 10-mesh metal net 156 having a diameter of 9 cm inside it. At the discharge duct part 157 at the bottom of the acrylic cylinder 154, provided is a 100-mesh metal net 158 having a diameter of 5 cm, through which the opened fibers were sieved under a reduced pressure of −60 mmH₂O (see FIG. 32 (b)). The weight K2 (unit, g) of the residue remaining on the 100-mesh metal net was measured, and the openability of the absorbent composite was evaluated according to the following formula:

Openability (%)=[(K1−K2)/K1]×100.

### (Determination of Recovery of Absorbent Composite)

An absorbent composite was cut into a square piece of 5 cm×5 cm, and put into a frame of the same size. The absorbent composite was compressed by applying thereto a pressure of 1 MPa on and below it over 10 minutes. While thus pressed, this was stored at a temperature of 25° C. and a humidity of 50% for 30 days. According to the above-mentioned method for thickness measurement, the thickness of the composite was measured just after compression and after 30 days, and the recovery of the composite was computed according to the formula mentioned below. Five samples were tested, and their data were averaged.

Recovery (%)=[(thickness in 30 days after compression−thickness just after compression)/(thickness just after compression)]×100.

### (Peeling Test of Absorbent Composite)

An absorbent composite was cut into a square piece of 5 cm×5 cm. With fingers, one outer substrate of the sample and the other substrate were picked up, and gradually peeled away. The peeling condition was checked with the naked eye.

### (Wet Peeling Test of Composite Having at Least Two Substrate Layers)

(1) An absorbent composite was cut into a square piece of 5 cm×5 cm.

(2) 10 ml of pure water was put into a laboratory dish of glass having a diameter of 10 cm.

(3) The absorbent composite was dipped in pure water in the dish for 10 minutes so as to absorb water.

(4) The outer substrate of one absorbent composite was picked up with the fingers of one hand and the other outer substrate of the other absorbent composite was picked up with the fingers of the other hand, and these were gradually peeled away.

(5) The peeling condition was checked with the naked eye.

(Determination of Artificial Urine Absorbing Speed of Diaper and Released Urine Amount from Diaper)

The diaper of Example 19 and Example 23 was cut into a rectangular piece of 40 cm×10 cm. The artificial urine absorbing speed of the diaper sample and the released urine amount from the sample were measured, using the same device as that used for measurement of the absorbent composite gel dropout percentage and according to the process mentioned below (see FIG. 25).

(a) A diaper 52 was put on a smooth face 51. An acrylic plate 55 (rectangular parallelepiped of 100 mm×100 mm×10 mm) was put on it. At the center of the acrylic plate 55, a cylinder 53 opened at its top and having an inner diameter of 40 mm was put, and 7 through-holes 54 each having a diameter of 5 mm were formed at nearly regular intervals in the area surrounded by the cylinder. The mass of the acrylic plate 55 was 150 g inclusive of the cylinder 53.

(b) A metallic disc 56 (mass, 1250 g) having a diameter of 100 mm, in which a hole 56A having a diameter of 45 mm was formed at the center thereof, was put on it through the cylinder 53.

(c) 25 ml of artificial urine was put into the cylinder 53 and absorbed by the diaper.

(d) The time taken before the urine was absorbed by the diaper was measured with a stopwatch, and it is the absorbing speed (second).

(e) After 10 minutes, the metallic disc 56 and the cylinder-fitted acrylic plate 55 were removed. At the position at which the acrylic plate 65 was on the diaper 52, 20 sheets of filter paper (Toyo Filter's ADVANTEC No. 424, 100×100 mm) were put as piled up thereon. Then, a weight of 4 kg having a bottom surface of 10 cm×10 cm square was put the filter paper. After 5 minutes, the weight was removed, and the weight of the filter paper was measured, and the amount of the artificial urine absorbed by the filter paper was determined and this is the released urine amount (g).

(f) The process of above (a) to (e) was repeated twice again, and the data were averaged.

TABLE 1

| | | | | Example | | | | | |
|---|---|---|---|---|---|---|---|---|---|
| Items | | | unit | 1 | 2 | 3 | 4 | 5 | 6 |
| Substrate | Conversion just before substrate formation | | % | Not tested | | | | | |
| Provision | Water content just before substrate provision | | % | | | | | | |
| Condition | Substrate provision starting time | | sec | | | | | | |
| | Pressure | | MPa | | | | | | |
| Polymerization | Polymerization Temperature | | ° C. | | | | | | |
| Condition | Water content after drying | | % | 5 | 5 | 5 | 5 | 5 | 5 |
| | Specific volume of fibrous substrate before | | ml/g | 50 | 50 | 50 | 50 | 50 | 50 |
| | polymerization | | | | | | | | |
| | Conversion at dropping on fibrous substrate | | % | 50 | 30/70 | 30/70 | 45 | 26/70 | 50 |
| | Unit weight of absorbent polymer | | g/m² | 300 | 300 | 300 | 300 | 300 | 300 |
| Morphology, | Absorbent | mean particle size | μm | 300 | 300 | 300 | 300 | 300 | 300 |
| Dimension | particles | | | | | | | | |
| | Web-like | morphology | — | Dispersion | Dispersion | Dispersion | Continuous | Con- | Con- |
| | absorbent | | | layer | layer | layer | layer | tinuous | tinuous |
| | polymer | | | | | | | layer | layer |
| | | thickness | μm | 150 | 150 | 150 | 150 | 150 | 150 |
| | | mean diameter | μm | 600 | 600 | 600 | No pores | No pores | No pores |
| | | mean pore size | μm | No pores | No pores | No pores | 600 | 600 | 600 |
| | | porosity | % | No pores | No pores | No pores | 40 | 40 | 40 |
| | Absorbent | thickness | μm | 900 | 900 | 900 | 950 | 950 | 950 |
| | composite | ratio by weight of bound | parts by | 90 | 100 | 95 | 85 | 33 | 93 |
| | | particles/web-like polymer | weight | | | | | | |

| | | | | |
|---|---|---|---|---|
| 1st stage | absorbent | mean particle size | μm | Omitted |
| composite | resin | morphology | — | |
| | aggregate | mean particle size | μm | |
| 2nd stage | absorbent | mean particle size | μm | |
| composite | resin | morphology | — | |
| | aggregate | mean particle size | μm | |

| | Ratio by weight of absorbent | — | No fibers | | | | | |
|---|---|---|---|---|---|---|---|---|
| | polymer/fibers | | | | | | | |
| Performance | Water-holding ability (CRC) | g/g | 35 | 36 | 34 | 35 | 35 | 35 |
| | Absorbent capacity under pressure (AUL: 20 g) | g/g | 25 | 25 | 25 | 25 | 25 | 25 |
| | Absorbent capacity under pressure (AUL: 50 g) | g/g | 20 | 20 | 20 | 20 | 20 | 20 |
| | Water-absorbing speed | sec | 18 | 18 | 18 | 20 | 20 | 20 |
| | Liquid-absorbing speed | sec | Not tested | | | | | |
| | Released amount | g | | | | | | |
| | Absorbent polymer dropout percentage | % | 0 | 0 | 0 | 0 | 0 | 0 |
| | Absorbent composite gel dropout percentage | % | 0 | 0 | 0 | 0 | 0 | 0 |
| | Gel dropout percentage after absorption | % | 18 | 18 | 18 | 18 | 18 | 18 |
| | Thickness | mm | Not tested | | | | | |
| | Density | g/m³ | | | | | | |
| | Openability | % | | | | | | |
| | Bending resistance | cm | 8.5 | 8.5 | 8.5 | 8 | 8 | 8 |
| | Recovery | % | Not tested | | | | | |
| | Peeling test | — | Out of | Out of | Out of | Out of | Out of | Out of |
| | | | definition | definition | definition | definition | definition | definition |
| | Wet peeling test | — | Out of | Out of | Out of | Out of | Out of | Out of |
| | | | definition | definition | definition | definition | definition | definition |
| Remarks | SAP form (web-like, bound particulate, | — | web-like + | web-like + | web-like + | web-like + | web-like + | web-like + |
| | bounded beads) | | bound | bound | bound | bound | bound | bound |
| | | | particulate | particulate | particulate | particulate | particulate | particulate |
| | Raising | — | Not raised | Not raised | Not raised | Not raised | Not raised | Not raised |
| | Substrate (one side, both sides) | — | One side | One side | One side | One side | One side | One side |
| | Nozzle protective liquid film | — | Not | Not formed | Not | Not formed | Not | Not |
| | | | formed | | formed | | formed | formed |
| | Nozzle shape | — | Counter- | Counter- | Counter- | Counter- | Counter- | Counter- |
| | | | facing | facing | facing | facing | facing | facing |

| | | | | Example | | | | | |
|---|---|---|---|---|---|---|---|---|---|
| Items | | | unit | 7 | 8 | 9 | 10 | 11 | 12 |
| Substrate | Conversion just before substrate formation | | % | 50 | 40 | 50 | 50 | 50 | 50 |
| Provision | Water content just before substrate provision | | % | 40 | 40 | 40 | 40 | 40 | 40 |
| Condition | Substrate provision starting time | | sec | 10 | 10 | 10 | 10 | 10 | 10 |
| | Pressure | | MPa | 0.005 | 0.005 | 0.005 | 0.005 | 0.005 | 0.1 |
| Polymerization | Polymerization Temperature | | ° C. | 50 | 40 | 50 | 50 | 50 | 50 |
| Condition | Water content after drying | | % | 5 | 5 | 5 | 5 | 5 | 5 |
| | Specific volume of fibrous substrate before | | ml/g | 50 | 50 | 50/50 | 50 | 50 | 50 |
| | polymerization | | | | | | | | |
| | Conversion at dropping on fibrous substrate | | % | 50 | 45 | 50 | 50 | 50 | 50 |
| | Unit weight of absorbent polymer | | g/m² | 300 | 300 | 300 | 300 | 300 | 300 |
| Morphology, | Absorbent | mean particle size | μm | 300 | 300 | 300 | 300 | 300 | 300 |
| Dimension | particles | | | | | | | | |
| | Web-like | morphology | — | Dispersion | Con- | Dispersion | Dispersion | Dispersion | Dispersion |
| | absorbent | | | layer | tinuous | layer | layer | layer | layer |
| | polymer | | | | layer | | | | |
| | | thickness | μm | 150 | 150 | 150 | 150 | 150 | 150 |
| | | mean diameter | μm | 600 | No pores | 600 | 600 | 600 | 600 |
| | | mean pore size | μm | No pores | 600 | No pores | No pores | No pores | — |
| | | porosity | % | No pores | 40 | No pores | No pores | No pores | — |
| | Absorbent | thickness | μm | 1200 | 1500 | 1300 | 1600 | 1200 | 1200 |
| | composite | ratio by weight of bound | parts by | 90 | 50 | 90 | 90 | 90 | 90 |
| | | particles/web-like polymer | weight | | | | | | |

| | | | | |
|---|---|---|---|---|
| 1st stage | absorbent | mean particle size | μm | Omitted |
| composite | resin | morphology | — | |
| | aggregate | mean particle size | μm | |
| 2nd stage | absorbent | mean particle size | μm | |
| composite | resin | morphology | — | |
| | aggregate | mean particle size | μm | |

| | Ratio by weight of absorbent | — | No fibers | | | | | |
|---|---|---|---|---|---|---|---|---|
| | polymer/fibers | | | | | | | |
| Performance | Water-holding ability (CRC) | g/g | 35 | 35 | 35 | 35 | 35 | 35 |
| | Absorbent capacity under pressure (AUL: 20 g) | g/g | 25 | 25 | 25 | 25 | 25 | 25 |
| | Absorbent capacity under pressure (AUL: 50 g) | g/g | 20 | 20 | 20 | 20 | 20 | 20 |
| | Water-absorbing speed | sec | Not tested | | | | | |
| | Liquid-absorbing speed | sec | Not tested | | | | | |
| | Released amount | g | | | | | | |
| | Absorbent polymer dropout percentage | % | 0 | 0 | 0 | 0 | 0 | 0 |
| | Absorbent composite gel dropout percentage | % | 0 | 0 | 0 | 0 | 0 | 0 |
| | Gel dropout percentage after absorption | % | 0 | 0 | 0 | 0 | 0 | 0 |
| | Thickness | mm | Not tested | | | | | |
| | Density | g/m³ | | | | | | |
| | Openability | % | | | | | | |
| | Bending resistance | cm | 8 | 8 | 8.5 | 7 | 8 | 8 |
| | Recovery | % | Not tested | | | | | |
| | Peeling test | — | Pulp | Pulp | Pulp | Pulp | Pulp | Pulp |
| | | | material | material | material | material | material | material |
| | | | disrupted | disrupted | disrupted | disrupted | disrupted | disrupted |
| | Wet peeling test | — | Pulp | Pulp | Pulp | Pulp | Pulp | Pulp |
| | | | material | material | material | material | material | material |
| | | | disrupted | disrupted | disrupted | disrupted | disrupted | disrupted |
| Remarks | SAP form (web-like, bound particulate, | — | web-like + | web-like + | web-like + | web-like + | web-like + | web-like + |
| | bounded beads) | | bound | bound | bound | bound | bound | bound |
| | | | particulate | particulate | particulate | particulate | particulate | particulate |
| | Raising | — | Not raised | Not raised | Not raised | Not raised | Not raised | Not raised |
| | Substrate (one side, both sides) | — | Both sides | Both sides | Both sides | Both sides | Both sides | Both sides |
| | Nozzle protective liquid film | — | Not formed | Not | Not formed | Not | Not | Not |
| | | | | formed | | formed | formed | formed |
| | Nozzle shape | — | Counter- | Counter- | Counter- | Counter- | Counter- | Counter- |
| | | | facing | facing | facing | facing | facing | facing |

| | | | | Example | | | | | |
|---|---|---|---|---|---|---|---|---|---|
| Items | | | unit | 13 | 14 | 15 | 16 | 17 | 18 |
| Substrate | Conversion just before substrate formation | | % | 50 | Not tested | | | 50 | 50 |
| Provision | Water content just before substrate provision | | % | 40 | | | | 40 | 40 |
| Condition | Substrate provision starting time | | sec | 10 | | | | 10 | 10 |
| | Pressure | | MPa | 0.005 | | | | 0.005 | 0.005 |
| Polymerization | Polymerization Temperature | | ° C. | 50 | | | | 50 | 50 |
| Condition | Water content after drying | | % | 5 | 5 | 5 | 5 | 5 | 5 |
| | Specific volume of fibrous substrate before | | ml/g | 50 | 100 | 80 | 70 | 100 | 100 |
| | polymerization | | | | | | | | |
| | Conversion at dropping on fibrous substrate | | % | 50 | 50 | 50 | 50 | 50 | 50 |
| | Unit weight of absorbent polymer | | g/m² | 300 | 300 | 300 | 300 | 300 | 300 |
| Morphology, | Absorbent | mean particle size | μm | 300 | 300 | 300 | 300 | 300 | 300 |
| Dimension | particles | | | | | | | | |
| | Web-like | morphology | — | Dispersion | Dispersion | Dispersion | Dispersion | Dispersion | Dispersion |
| | absorbent | | | layer | layer | layer | layer | layer | layer |
| | polymer | thickness | μm | 150 | 150 | 150 | 150 | 150 | 150 |
| | | mean diameter | μm | 600 | 600 | 600 | 600 | 600 | 600 |
| | | mean pore size | μm | No pores | No pores | No pores | No pores | No pores | No pores |
| | | porosity | % | No pores | No pores | No pores | No pores | No pores | No pores |
| | Absorbent | thickness | μm | 900 | 900 | 900 | 900 | 900 | 900 |
| | composite | ratio by weight of bound | parts by | 90 | 90 | 90 | 90 | 90 | 90 |
| | | particles/web-like polymer | weight | | | | | | |

| | | | | |
|---|---|---|---|---|
| 1st stage | absorbent | mean particle size | μm | Omitted |
| composite | resin | morphology | — | |
| | aggregate | mean particle size | μm | |
| 2nd stage | absorbent | mean particle size | μm | |
| composite | resin | morphology | — | |
| | aggregate | mean particle size | μm | |

| | Ratio by weight of absorbent | — | No fibers | | | | | |
|---|---|---|---|---|---|---|---|---|
| | polymer/fibers | | | | | | | |
| Performance | Water-holding ability (CRC) | g/g | 35 | 35 | 35 | 35 | 35 | 35 |
| | Absorbent capacity under pressure (AUL: 20 g) | g/g | 25 | 25 | 25 | 25 | 25 | 25 |
| | Absorbent capacity under pressure (AUL: 50 g) | g/g | 20 | 20 | 20 | 20 | 20 | 20 |
| | Water-absorbing speed | sec | Not tested | 15 | 15 | 15 | Not tested | |
| | Liquid-absorbing speed | sec | Not tested | | | | | |
| | Released amount | g | | | | | | |
| | Absorbent polymer dropout percentage | % | 0 | 0 | 0 | 0 | 0 | 0 |
| | Absorbent composite gel dropout percentage | % | 0 | 0 | 0 | 0 | 0 | 0 |
| | Gel dropout percentage after absorption | % | 0 | 5 | 5 | 5 | 0 | 0 |
| | Thickness | mm | Not tested | | | | | |
| | Density | g/m³ | | | | | | |
| | Openability | % | | | | | | |
| | Bending resistance | cm | 8.5 | 8.5 | 8.5 | 8.5 | 8.5 | 8.5 |
| | Recovery | % | Not tested | | | | | |
| | Peeling test | — | Nonwoven | Out of | Out of | Out of | Pulp | Nonwoven |
| | | | fabric | definition | definition | definition | material | fabric |
| | | | disrupted | | | | disrupted | disrupted |
| | Wet peeling test | — | Nonwoven | Out of | Out of | Out of | Pulp | Nonwoven |
| | | | fabric/ | definition | definition | definition | material | fabric/ |
| | | | polymer | | | | disrupted | polymer |
| | | | interface | | | | | interface |
| | | | de- | | | | | delaminated |
| | | | laminated | | | | | |
| Remarks | SAP form (web-like, bound particulate, | — | web-like + | web-like + | web-like + | web-like + | web-like + | web-like + |
| | bounded beads) | | bound | bound | bound | bound | bound | bound |
| | | | particulate | particulate | particulate | particulate | particulate | particulate |
| | Raising | — | Not raised | Raised | Raised | Raised | Raised | Raised |
| | Substrate (one side, both sides) | — | Both sides | One side | One side | One side | Both sides | Both sides |
| | Nozzle protective liquid film | — | Not | Not | Not | Not formed | Not | Not formed |
| | | | formed | formed | formed | | formed | |
| | Nozzle shape | — | Counter- | Counter- | Counter- | Counter- | Counter- | Counter- |
| | | | facing | facing | facing | facing | facing | facing |

| | | | | | Example | | | | |
|---|---|---|---|---|---|---|---|---|---|
| Items | | | | unit | 19 | 20 | 21 | 22 | 23 |
| Substrate | Conversion just before substrate formation | | | % | Omitted | | | | |
| Provision | Water content just before substrate provision | | | % | | | | | |
| Condition | Substrate provision starting time | | | sec | | | | | |
| | Pressure | | | MPa | | | | | |
| Polymerization | Polymerization Temperature | | | ° C. | | | | | |
| Condition | Water content after drying | | | % | 5 | 5 | 5 | 5 | 5 |
| | Specific volume of fibrous substrate before | | | ml/g | 50 | 50 | 50 | 50 | 50 |
| | polymerization | | | | | | | | |
| | Conversion at dropping on fibrous substrate | | | % | 60 | 60 | 60 | 60 | 60 |
| | Unit weight of absorbent polymer | | | g/m² | 300 | 200 | 300 | 200 | 300 |
| Morphology, | Absorbent | mean particle size | | μm | Not measured | | | | |
| Dimension | particles | | | | | | | | |
| | Web-like | morphology | | — | | | | | |
| | absorbent | thickness | | μm | | | | | |
| | polymer | mean diameter | | μm | | | | | |
| | | mean pore size | | μm | | | | | |
| | | porosity | | % | | | | | |
| | Absorbent | thickness | | μm | | | | | |
| | composite | ratio by weight of bound particles/ | | parts by | | | | | |
| | | web-like polymer | | weight | | | | | |
| | 1st stage | absorbent | mean particle size | μm | 400 | 400 | 400 | 400 | 400 |
| | composite | resin | morphology | — | Nearly | Nearly | Nearly | Nearly | Nearly |
| | | | | | spherical | spherical | spherical | spherical | spherical |
| | | aggregate | mean particle size | μm | 850 | 850 | 850 | 850 | 850 |
| | 2nd stage | absorbent | mean particle size | μm | 400 | Omitted | 400 | 400 | Omitted |
| | composite | resin | morphology | — | Nearly | | Nearly | Nearly | |
| | | | | | spherical | | spherical | spherical | |
| | | aggregate | mean particle size | μm | 850 | | 850 | 850 | |

| | Ratio by weight of absorbent | — | 86/14 | 75/25 | 86/14 | 75/25 | 86/14 |
|---|---|---|---|---|---|---|---|
| | polymer/fibers | | | | | | |
| Performance | Water-holding ability (CRC) | g/g | 34 | 35 | 34 | 34 | 34 |
| | Absorbent capacity under pressure (AUL: 20 g) | g/g | Not tested | | | | |
| | Absorbent capacity under pressure (AUL: 50 g) | g/g | | | | | |
| | Water-absorbing speed | sec | | | | | |
| | Liquid-absorbing speed | sec | 6 | Not tested | | | 6 |
| | Released amount | g | 2.3 | | | | 3 |
| | Absorbent polymer dropout percentage | % | 0 | | | | 4 |
| | Absorbent composite gel dropout percentage | % | 0 | | | | 15 |
| | Gel dropout percentage after absorption | % | Not tested | | | | |
| | Thickness | mm | 0.95 | 0.8 | 1.03 | 0.68 | 0.78 |
| | Density | g/m³ | 0.37 | 0.29 | 0.34 | 0.46 | 0.36 |
| | Openability | % | 100 | 100 | 100 | 100 | 100 |
| | Bending resistance | cm | 7.5 | 8 | 7.5 | 8.5 | 8 |
| | Recovery | % | 9 | 12 | 18 | 20 | 11 |
| | Peeling test | — | Omitted | | | | |
| | Wet peeling test | — | | | | | |
| Remarks | SAP form (web-like, bound particulate, | — | web-like + | web-like + | web-like + | web-like + | web-like + |
| | bounded beads) | | bound | bound | bound | bound | bound |
| | | | particulate | particulate | particulate | particulate | particulate |
| | Raising | — | Raised | Raised | Raised | Raised | Raised |
| | Substrate (one side, both sides) | — | One side | One side | One side | One side | One side |
| | Nozzle protective liquid film | — | Not formed | Not formed | Not formed | Not formed | Not formed |
| | Nozzle shape | — | Counter- | Counter- | Counter- | Counter- | Counter- |
| | | | facing | facing | facing | facing | facing |

| | | | | Example | | | Comparative Example | |
|---|---|---|---|---|---|---|---|---|
| Items | | | unit | 24 | 25 | 26 | 1 | 2 |
| Substrate | Conversion just before substrate formation | | % | Not tested | | | Not tested | 85 |
| Provision | Water content just before substrate provision | | % | | | | | 15 |
| Condition | Substrate provision starting time | | sec | | | | | 3600 |
| | Pressure | | MPa | | | | | 0.005 |
| Polymerization | Polymerization Temperature | | ° C. | | | | | 90 |
| Condition | Water content after drying | | % | 5 | 5 | 5 | 5 | 5 |
| | Specific volume of fibrous substrate before | | ml/g | 50 | 50 | 50 | 50 | 50 |
| | polymerization | | | | | | | |
| | Conversion at dropping on fibrous substrate | | % | 50 | 50 | 62 | 90 | 50 |
| | Unit weight of absorbent polymer | | g/m² | 300 | 300 | 300 | 300 | 300 |
| Morphology, | Absorbent | mean particle size | μm | 300 | 300 | 300 | 300 | 300 |
| Dimension | particles | | | | | | | |
| | Web-like | morphology | — | Dispersion | Dispersion | Continuous | No web | Dispersion |
| | absorbent | | | layer | layer | layer | | layer |
| | polymer | thickness | μm | 150 | 150 | 150 | | 150 |
| | | mean diameter | μm | 600 | 600 | 600 | | 600 |
| | | mean pore size | μm | No pores | No pores | No pores | | No pores |
| | | porosity | % | No pores | No pores | No pores | | No pores |
| | Absorbent | thickness | μm | 900 | 900 | 900 | 800 | 1200 |
| | composite | ratio by weight of bound particles/ | parts by | 90 | 90 | 103 | Web | 90 |
| | | web-like polymer | weight | | | | | |

| | | | | | |
|---|---|---|---|---|---|
| 1st stage | absorbent | mean particle size | μm | Omitted | Omitted |
| composite | resin | morphology | — | | |
| | aggregate | mean particle size | μm | | |
| 2nd stage | absorbent | mean particle size | μm | | |
| composite | resin | morphology | — | | |
| | aggregate | mean particle size | μm | | |

| | Ratio by weight of absorbent | — | Not measured | | | Not measured | |
|---|---|---|---|---|---|---|---|
| | polymer/fibers | | | | | | |
| Performance | Water-holding ability (CRC) | g/g | 35 | 35 | 30 | 35 | 35 |
| | Absorbent capacity under pressure (AUL: 20 g) | g/g | 25 | 25 | 25 | 22 | 25 |
| | Absorbent capacity under pressure (AUL: 50 g) | g/g | 20 | 20 | 20 | 18 | 20 |
| | Water-absorbing speed | sec | 18 | 18 | 18 | 25 | Not tested |
| | Liquid-absorbing speed | sec | Not tested | | | Not tested | |
| | Released amount | g | | | | | |
| | Absorbent polymer dropout percentage | % | 0 | 0 | 0 | 0 | 0 |
| | Absorbent composite gel dropout percentage | % | 0 | 0 | 0 | 15 | 0 |
| | Gel dropout percentage after absorption | % | 18 | 18 | 19 | 90 | 90 |
| | Thickness | mm | Not tested | | | Not tested | |
| | Density | g/m³ | | | | | |
| | Openability | % | | | | | |
| | Bending resistance | cm | 8.5 | 8.5 | 8.5 | Undetected | 7 |
| | Recovery | % | Not tested | | | Not tested | |
| | Peeling test | — | Out of | Out of | Out of | Out of | Pulp/polymer |
| | | | definition | definition | definition | definition | interface |
| | | | | | | | delaminated |
| | Wet peeling test | — | Out of | Out of | Out of | Out of | Pulp/polymer |
| | | | definition | definition | definition | definition | interface |
| | | | | | | | delaminated |
| Remarks | SAP form (web-like, bound particulate, | — | web-like + | web-like + | web-like + | bound | web-like + |
| | bounded beads) | | bound | bound | bound | particulate | bound |
| | | | particulate | particulate | particulate | | particulate |
| | Raising | — | Not raised | Not raised | Not raised | Not raised | Not raised |
| | Substrate (one side, both sides) | — | One side | One side | One side | One side | Both sides |
| | Nozzle protective liquid film | — | Formed | Formed | Formed | Not formed | Not formed |
| | Nozzle shape | — | Double | Double | Double | Counter- | Counter- |
| | | | concentric | concentric | concentric | facing | facing |
| | | | centrifugal | centrifugal | centrifugal | | |
| | | | jet | jet | jet | | |
| | | | nozzle | nozzle | nozzle | | |

| | | | Comparative Example | | | | |
|---|---|---|---|---|---|---|---|
| Items | | unit | 3 | 4 | 5 | 6 | 7 |
| Substrate | Conversion just before substrate formation | % | 50 | 8 | Omitted | | |
| Provision | Water content just before substrate provision | % | 90 | 45 | | | |
| Condition | Substrate provision starting time | sec | 20 | 10 | | | |
| | Pressure | MPa | 0.005 | 0.005 | | | |
| Polymerization | Polymerization Temperature | ° C. | 50 | 5 | | | |
| Condition | Water content after drying | % | 5 | 5 | 5 | 5 | 5 |
| | Specific volume of fibrous substrate before | ml/g | 50 | 50 | 50 | 100 | 100 |
| | polymerization | | | | | | |
| | Conversion at dropping on fibrous substrate | % | 45 | 8 | 30 | 0 | 0 |
| | Unit weight of absorbent polymer | g/m² | 300 | 300 | 300 | 100 | 300 |

| | | | | | | |
|---|---|---|---|---|---|---|
| Morphology, | Absorbent | mean particle size | μm | 300 | 300 | Not measured |
| Dimension | particles | | | | | |
| | Web-like | morphology | — | Dispersion | Continuous | |
| | absorbent | | | layer | layer | |
| | polymer | thickness | μm | 150 | 150 | |
| | | mean diameter | μm | 600 | No pores | |
| | | mean pore size | μm | No pores | 600 | |
| | | porosity | % | No pores | 40 | |
| | Absorbent | thickness | μm | 1200 | 1500 | |
| | composite | ratio by weight of bound particles/ | parts by | 90 | 50 | |
| | | web-like polymer | weight | | | |

| | | | | | | |
|---|---|---|---|---|---|---|
| 1st stage | absorbent | mean particle size | μm | Omitted | 850 | Omitted |
| composite | resin | morphology | — | | Nearly | |
| | | | | | spherical | |
| | aggregate | mean particle size | μm | | Undetected(*1) | |
| 2nd stage | absorbent | mean particle size | μm | | Omitted | |
| composite | resin | morphology | — | | | |
| | aggregate | mean particle size | μm | | | |

| | Ratio by weight of absorbent | — | Not measured | | 75/25 | Not measured | |
|---|---|---|---|---|---|---|---|
| | polymer/fibers | | | | | | |
| Performance | Water-holding ability (CRC) | g/g | 35 | 35 | 28 | 35 | 28 |
| | Absorbent capacity under pressure (AUL: 20 g) | g/g | 25 | 20 | Not tested | 25 | 20 |
| | Absorbent capacity under pressure (AUL: 50 g) | g/g | 20 | 10 | | 20 | 10 |
| | Water-absorbing speed | sec | Not tested | | | 120 | 180 |
| | Liquid-absorbing speed | sec | Not tested | | | Not tested | |
| | Released amount | g | | | | | |
| | Absorbent polymer dropout percentage | % | 0 | 0 | | | |
| | Absorbent composite gel dropout percentage | % | 0 | 0 | | | |
| | Gel dropout percentage after absorption | % | 90 | 80 | | 5 | 20 |
| | Thickness | mm | Not tested | | 2.55 | Not tested | |
| | Density | g/m³ | | | 0.05 | | |
| | Openability | % | | | 1(*2) | | |
| | Bending resistance | cm | 7 | 7 | Undetected(*3) | 8 | Un- |
| | | | | | | | detected(*3) |
| | Recovery | % | Not tested | | 10 | Not tested | |
| | Peeling test | — | Pulp/polymer | Pulp/polymer | Not tested | | |
| | | | interface | interface | | | |
| | | | delaminated | delaminated | | | |
| | Wet peeling test | — | Pulp/polymer | Pulp/polymer | | | |
| | | | interface | interface | | | |
| | | | delaminated | delaminated | | | |
| Remarks | SAP form (web-like, bound particulate, | — | web-like + | web-like + | web-like + | web-like | web-like |
| | bounded beads) | | bound | bound | bound | | |
| | | | particulate | particulate | particulate | | |
| | Raising | — | Not raised | Not raised | Raised | Not raised | Not raised |
| | Substrate (one side, both sides) | — | Both sides | Both sides | One side | One side | One side |
| | Nozzle protective liquid film | — | Not formed | Not formed | Not formed | Not | Not formed |
| | | | | | | formed | |
| | Nozzle shape | — | Counter- | Counter- | Counter- | Counter- | Counter- |
| | | | facing | facing | facing | facing | facing |

(*1)The aggregates were tabular, and their particle size could not be measured.
(*2)The web-like particles were broken to give powder.
(*3)This was not soft, and could not form a heart loop but broke halfway.

### INDUSTRIAL APPLICABILITY

The absorbent composite of the invention has a high absorbent speed, in which an absorbent polymer is uniformly fixed to fibers throughout before and after absorption, and the composite has suitable softness. According to the production method for an absorbent composite of the invention, the absorbent composite having such characteristics can be efficiently produced. In particular, in the absorbent composite of the invention, an adhesive, sticky and moisture-absorbing absorbent polymer is covered with a substrate, and therefore the composite is prevented from adhering to contact parts and slide parts such as a winding-up roller, and its in-line producibility is good.

Accordingly, the absorbent composite of the invention is suitable to production of sanitary materials such as paper diapers and sanitary protections; industrial materials necessary for absorbing and holding waste water; and agricultural materials for freshness holders for vegetables and others and for water holders. For the production method for such an absorbent composite of the invention, industrial production systems can be used, and the invention is suitable to industrial mass-production. Accordingly, the industrial applicability of the invention is great.

1. An absorbent composite comprising a bound particulate absorbent polymer in which two or more nearly spherical absorbent polymer particles are bound to each other and a web-like absorbent polymer, wherein the bound particulate absorbent polymer and the web-like absorbent polymer are bound to a substrate.

2. The absorbent composite according to claim 1, wherein the substrate is bound to both surfaces of the layer comprising the bound particulate absorbent polymer and the web-like absorbent polymer.

3. The absorbent composite according to claim 2, wherein at least one substrate is a fibrous substrate.

4. The absorbent composite according to claim 1, which has an absorbing speed of from 1 to 100 seconds and has a gel dropout percentage after absorption of at most 60% by weight.

5. The absorbent composite according to claim 1, wherein the particle size of the nearly spherical absorbent polymer is from 20 to 5000 μm.

6. The absorbent composite according to claim 1, wherein the thickness of the web-like absorbent polymer is from 50 to 1000 μm.

7. The absorbent composite according to claim 1, wherein a web-like absorbent polymer having a size of from 200 to 50000 μm is dispersed on the substrate like islands.

8. The absorbent composite according to claim 1, wherein a continuous layer of a web-like absorbent polymer having a pore size of from 100 to 50000 μm and a porosity of from 10 to 80% by area is provided on the substrate.

9. The absorbent composite according to claim 1, wherein the substrate is a fibrous substrate.

10. A method for producing an absorbent composite, which comprises polymerizing a polymerizable monomer to give an absorbent polymer and a polymerization initiator in a vapor phase in a mode of droplet polymerization, and contacting it with a substrate while the conversion of the polymerizable monomer is at most 40%.

11. The production method for an absorbent composite according to claim 10, which comprises polymerizing a polymerizable monomer to give an absorbent polymer and a polymerization initiator in a vapor phase in a mode of droplet polymerization, and contacting it with a substrate while the conversion of the polymerizable monomer is at most 40%, and contacting it with a substrate while the conversion of the polymerizable monomer is more than 40%.

12. The production method for an absorbent composite according to claim 11, which comprises polymerizing a polymerizable monomer to give an absorbent polymer and a polymerization initiator in a vapor phase in a mode of droplet polymerization, and contacting it with a first substrate while the conversion of the polymerizable monomer is at most 40% and contacting it with the first substrate while the conversion of the polymerizable monomer is more than 40%, and further comprises, after these steps, contacting it with a second substrate while the mean conversion of the polymerizable monomer is from 10 to 80%.

13. The production method for an absorbent composite according to claim 12, which comprises polymerizing a polymerizable monomer to give an absorbent polymer and a polymerization initiator in a vapor phase in a mode of droplet polymerization, and contacting it with a first substrate while the conversion of the polymerizable monomer is at most 40% and contacting it with the first substrate while the conversion of the polymerizable monomer is more than 40%, and further comprises, after these steps, contacting it with a second substrate while the mean conversion of the polymerizable monomer is from 10 to 80%, and pressing the first substrate and the second substrate under a pressure of from 0.0001 to 1 MPa with their polymerizable monomer contact surfaces facing each other.

14. The production method for an absorbent composite according to claim 10, wherein the substrate is a raised fibrous substrate.

15. The production method for an absorbent composite according to claim 10, in which the substrate is a fiber deposition layer and which comprises depositing opened fibers on the substrate after single particles and/or aggregates of an absorbent composite have been formed thereon.

16. The production method for an absorbent composite according to claim 10, wherein the substrate is a deposition layer of opened fibers and the application of the polymerizable monomer droplets under polymerization onto the substrate is repeated twice or more.

17. An absorbent article formed with an absorbent composite of claim 1.

18. A sanitary material formed with an absorbent composite of claim 1.

19. A diaper formed with an absorbent composite of claim 1.

20. An industrial material formed with an absorbent composite of claim 1.

21. An agricultural material formed with an absorbent composite of claim 1.

22. A polymerization nozzle for use in a method of producing an absorbent composite that comprises polymerizing a polymerizable monomer to give an absorbent polymer and a polymerization initiator in a vapor phase in a mode of droplet polymerization, and contacting it with a substrate while the conversion of the polymerizable monomer is at most 40%, in which the wall thickness of the tip to constitute the nozzle orifice is at most 10 mm.

23. The polymerization nozzle according to claim 22, wherein the tip of the polymerization nozzle is tapered in the direction of the tip.

24. The polymerization nozzle according to claim 22, wherein the tip of the polymerization nozzle is tapered in the direction of the tip, and the crossing angle of the taper is at most 160°.

25. A polymerization nozzle provided with a mechanism of forming a liquid film on the outer wall surface of the polymerization nozzle.

26. The polymerization nozzle according to claim 25, which is provided with a mechanism capable of forming the liquid film by continuously or intermittently applying a liquid toward the nozzle orifice of the polymerization nozzle, from outside the nozzle orifice.

27. The polymerization nozzle according to claim 25, which is provided with a mechanism capable of forming the liquid film by spraying a liquid toward the outer wall surface of the polymerization nozzle.

28. The polymerization nozzle according to claim 25, which is provided with a mechanism capable of forming the liquid film by spraying a liquid toward the upper outer wall surface of the polymerization nozzle above the nozzle orifice.

29. The polymerization nozzle according to claim 25, which has openings for leading the liquid for forming the liquid film toward outside the polymerization nozzle from inside the nozzle.

30. The polymerization nozzle according to claim 25, wherein a liquid capable of dissolving or swelling the absorbent polymer is used as the liquid for forming the liquid film.

31. The polymerization nozzle according to claim 25, wherein a liquid having a high affinity for any one of the liquids used in polymerization, as the liquid for forming the liquid film.
